(19) United States
(12) Patent Application Publication
(10) Pub. No.: US 2026/0191937 A1
(43) Pub. Date: Jul. 9, 2026

(54) AGENTS, COMPOSITIONS AND METHODS FOR MODULATING IMMUNITY

(71) Applicant: The University of Queensland, St. Lucia (AU)

(72) Inventors: Matthew Sweet, St. Lucia (AU); Kaustav Das Gupta, St. Lucia (AU); Divya Ramnath, St. Lucia (AU); Chengzhong Yu, St. Lucia (AU); Yue Wang, St. Lucia (AU)

(21) Appl. No.: 19/132,127

(22) PCT Filed: Nov. 28, 2023
(86) PCT No.: PCT/AU2023/051221

§ 371 Date: May 22, 2025

(30) Foreign Application Priority Data
Nov. 28, 2022 (AU) 2022903606

(51) Int. Cl. A61K 38/44; A61K 9/127; A61K 9/51; A61K 31/7105; A61K 31/711; A61K 45/06; A61P 29/00; A61P 37/06

(52) U.S. Cl. CPC A61K 38/443; A61K 9/127; A61K 9/5123; A61K 9/5146; A61K 31/7105; A61K 31/711; A61K 45/06; A61P 29/00; A61P 37/06; C12Y 101/01044

(57) ABSTRACT

Disclosed are agents, compositions and methods for treating, reducing or inhibiting, or slowing the progression of, unwanted or undesirable immune responses including pro-inflammatory responses. More particularly, the present disclosure relates to a therapeutic agent, therapeutic combinations and their use in compositions and methods for inhibiting or reducing pro-inflammatory activity of immune cells such as antigen-presenting cells (APC), wherein the therapeutic agent comprises D-ribulose-5-phosphate (RL5P), a RL5P analog, a RL5P analog-producing agent and/or a RL5P-producing agent, such as one selected from 6-phosphogluconate dehydrogenase (6PGD) and a nucleic acid molecule from which 6PGD is producible, and wherein the therapeutic combination comprises the therapeutic agent and an HDAC7-producing agent.

### RELATED APPLICATION

This application claims priority to Australian Provisional Patent Application No. 2022903606 entitled “Agents, compositions and methods for modulating immunity” filed 28 Nov. 2022, the contents of which are incorporated herein by reference in their entirety.

### FIELD

This disclosure relates generally to agents, compositions and methods for treating, reducing or inhibiting, or slowing the progression of, unwanted or undesirable immune responses including pro-inflammatory responses. More particularly, the present disclosure relates to a therapeutic agent, therapeutic combinations and their use in compositions and methods for inhibiting or reducing pro-inflammatory activity of immune cells such as antigen-presenting cells (APC), wherein the therapeutic agent comprises D-ribulose-5-phosphate (RL5P), a RL5P analog, a RL5P analog-producing agent and/or a RL5P-producing agent, such as one selected from 6-phosphogluconate dehydrogenase (6PGD) and a nucleic acid molecule from which 6PGD is producible, and wherein the therapeutic combination comprises the therapeutic agent and an HDAC7-producing agent.

### BACKGROUND

Inflammation is the body's natural defensive response to an insult (e.g., injury, infection, allergens, and/or toxins), with the immune system playing a central role in orchestrating this process. The immune system can be divided into two types: the innate and adaptive immune systems. The innate immune system is the first, non-specific (antigen-independent) defense for fighting against, e.g., an invading pathogen or foreign body, where the adaptive immune system is the secondary, antigen-specific defense, which creates immunological memory after the initial innate response to a specific pathogen. During the innate immune response, immune cells are recruited to the site of injury or infection. The recruitment and infiltration of immune cells, along with the release of cytokines and other pro-inflammatory mediators, results in inflammation, which may cause redness, swelling, fever, and pain. Inflammation generally decreases once the injury or infection is resolved. However, in cases of severe or persistent infection or injury, or cases where an abnormal adaptive immune response is mounted against “self” antigens (e.g., autoimmune processes), this may lead to a chronic inflammatory response. In other words, inflammation can occur in the short-term (acute) or long-term (chronic), depending on the nature and severity of the immunological insult.

Inflammation can play a major role in the pathology of immune-mediated diseases. Current available treatments used to alleviate both acute and chronic inflammation include the use of non-steroidal anti-inflammatory drugs (NSAIDs) and corticosteroids. Due to their great therapeutic potential, NSAIDs and corticosteroids are widely used, both intermittently and in the longer term. However, their use is not without risks. For example, long-term use of such anti-inflammatory agents can result in significant side effects, such as gastrointestinal irritation and bleeding, bone loss, and fluid retention.

In view of the large number of diseases and conditions associated with inflammation, along with the limitations of the anti-inflammatory treatments currently available, there is a need for the development of improved or alternative anti-inflammatory treatments or preventatives.

### SUMMARY

The present disclosure stems in part from the unexpected finding that 6-phosphogluconate dehydrogenase (6PGD), an enzyme of the pentose phosphate pathway (PPP), as well as ribulose 5-phosphate (RL5P), the biochemical product of 6PGD, suppresses specific inflammatory responses in macrophages. In particular, it has been determined that 6PGD and RL5P inhibit production of pro-inflammatory cytokines, including IL-1β, TNF and likely others. Based on these findings, the present inventors consider that the anti-inflammatory properties of 6PGD and RL5P can be taken advantage of in order to obtain beneficial therapeutic outcomes for reducing inflammation, particularly in immune cells such as antigen-presenting cells (e.g., macrophages) where the 6PGD-RL5P pathway is active. In inflammatory states, it is desirable to reduce the production of pro-inflammatory cytokines, including IL-1β and TNF because such cytokines are involved in the up-regulation of inflammatory reactions including the stimulation, recruitment and proliferation of immune cells.

From the forgoing, it is proposed, in accordance with the present disclosure, that contacting antigen-presenting cells with an agent comprising RL5P, a RL5P analog, a RP5P-analog-producing agent and/or a RL5P-producing agent (e.g., one selected from 6PGD and a nucleic acid molecule from which 6PGD is producible) can assist in preventing or treating inflammation.

Accordingly, in one aspect, the present disclosure provides methods for inhibiting or reducing pro-inflammatory activity of an antigen-presenting cell. These methods comprise, consist or consist essentially of contacting the antigen-presenting cell with a therapeutic agent, the therapeutic agent comprising D-ribulose-5-phosphate (RL5P), a RL5P analog, a RL5P analog-producing agent and/or a RL5P-producing agent.

In some embodiments, the RL5P-producing agent is 6-phosphogluconate dehydrogenase (6PGD) and/or a nucleic acid molecule from which 6PGD is producible.

The antigen-presenting cell may be selected from a macrophage, a dendritic cell and a B-cell. In specific embodiments, the antigen-presenting cell is a macrophage.

The pro-inflammatory activity may comprise the production and/or release of a pro-inflammatory mediator. In some embodiments, the pro-inflammatory mediator is a pro-inflammatory cytokine, illustrative examples of which include IL-1β and TNF.

The therapeutic agent may comprise a particle that is capable of being taken up by the antigen-presenting cell. For example, the particle may be a nanoparticle or microparticle. In specific embodiments, the particle is selected from a liposome, a lipid-based particle, a lipid carrier such as a lipidoid, a lipoplex, a polymeric particle, an inorganic particle, an inorganic particle coated with polymer or lipid, a micelle, a filomicelle, an exosome, a peptide carrier, a lipoprotein, a lipid-coated bubble, a polymersome, a niosome, a nanotube, a carbon nanoassembly, a paramagnetic particle, a ferromagnetic particle, a microvesicle, a dendrimer, a hyperbranched polymer and a conjugate.

The therapeutic agent may comprise a nucleic acid molecule from which 6PGD is producible, wherein the nucleic acid molecule comprises, consists or consists essentially of a 6PGD coding sequence. In some embodiments, the nucleic acid molecule comprises mRNA. In other embodiments, the nucleic acid molecule comprises DNA. In illustrative examples of this type, the DNA coding sequence may be operably connected to a regulatory element that is operable in the antigen-presenting cell.

In some embodiments, the therapeutic agent is an agent that targets an antigen-presenting cell.

In some embodiments, the therapeutic agent comprises an antigen-presenting cell-targeting moiety.

It has also been found unexpectedly that the activity of 6PGD may be enhanced by a histone deacetylase 7 (HDAC7) polypeptide, including HDAC7 polypeptides lacking HDAC7 deacetylase activity and, based on the results presented herein, is predicted to lead to greater inhibition or reduction of antigen-presenting cell pro-inflammatory activity. Accordingly, in some embodiments, the methods for inhibiting or reducing pro-inflammatory activity of an antigen-presenting cell further comprise contacting the antigen-presenting cell with an HDAC7 polypeptide-producing agent (e.g., an HDAC7 polypeptide or a nucleic acid molecule from which an HDAC7 polypeptide is producible).

Based on this finding, it is proposed that an HDAC7 polypeptide-producing agent and the therapeutic agent disclosed herein may be co-delivered to pro-inflammatory immune cells generally to inhibit or reduce their pro-inflammatory activity. Accordingly, in another aspect, methods are disclosed for inhibiting or reducing pro-inflammatory activity of an immune cell, wherein the methods comprise, consist or consist essentially of contacting the immune cell with a therapeutic agent and an HDAC7 polypeptide-producing agent (e.g., an HDAC7 polypeptide or a nucleic acid molecule from which an HDAC7 polypeptide is producible), wherein the therapeutic agent comprises D-ribulose-5-phosphate (RL5P), a RL5P analog, a RL5P analog-producing agent and/or a RL5P-producing agent.

In another aspect of the present disclosure, therapeutic agents are provided, which comprise, consist or consist essentially of D-ribulose-5-phosphate (RL5P), a RL5P analog, a RL5P analog-producing agent and/or a RL5P-producing agent.

In some embodiments, the RL5P-producing agent is 6-phosphogluconate dehydrogenase (6PGD) and/or a nucleic acid molecule from which 6PGD is producible.

The therapeutic agent comprises and/or may be a particle that is capable of being taken up by an antigen-presenting cell. In some embodiments, the therapeutic agent comprises a particle such as a nanoparticle or microparticle. In some of the same and other embodiments, the particle is selected from a liposome, a lipid-based particle, a polymeric particle, an inorganic particle, an inorganic particle coated with polymer or lipid, a micelle, a filomicelle, an exosome, a lipoprotein, a lipid-coated bubble, a polymersome, a niosome, a carbon nanoassembly, a paramagnetic particle, a ferromagnetic particle, a microvesicle, a dendrimer, and a hyperbranched polymer.

The therapeutic agent may comprise a nucleic acid molecule from which 6PGD is producible, wherein the nucleic acid molecule comprises, consists or consists essentially of a 6PGD coding sequence. In some embodiments, the nucleic acid molecule comprises mRNA. In other embodiments, the nucleic acid molecule comprises DNA. In representative examples of this type, the DNA coding sequence may be operably connected to a regulatory element that is operable in the antigen-presenting cell.

In some embodiments, the therapeutic agent targets an antigen-presenting cell.

In some embodiments, the therapeutic agent comprises an antigen-presenting cell-targeting moiety.

Another aspect of the present disclosure provides a therapeutic combination comprising, consisting or consisting essentially of a therapeutic agent and an HDAC7 polypeptide-producing agent as broadly described above and elsewhere herein.

In some embodiments, one or both of the therapeutic agent and the HDAC7 polypeptide-producing agent are comprised or are otherwise associated with one or more particles. For example, the therapeutic agent and the HDAC7 polypeptide-producing agent may be comprised or otherwise associated with the same particle or with different particles, which are suitably capable of being taken up by an immune cell.

Disclosed herein in another aspect are pharmaceutical compositions suitably for use in inhibiting or reducing pro-inflammatory activity of an antigen-presenting cell, or for treating inflammation. These pharmaceutical compositions generally comprise a therapeutic agent as broadly described above and elsewhere herein and a pharmaceutically acceptable carrier, diluent or excipient.

In a related aspect, pharmaceutical compositions are provided, which are suitably for use in inhibiting or reducing pro-inflammatory activity of an immune cell, or for treating inflammation. These pharmaceutical compositions generally comprise a therapeutic combination as broadly described above and elsewhere herein and a pharmaceutically acceptable carrier, diluent or excipient.

In a further aspect, methods for treating, preventing, inhibiting or reducing, or slowing the progression of, inflammation in a subject are disclosed. These methods generally comprise, consist or consist essentially of administering to the subject an effective amount of a therapeutic agent or composition as broadly described above and elsewhere herein.

In a related aspect, methods are disclosed for treating, preventing, inhibiting or reducing, or slowing the progression of, inflammation in a subject. These methods generally comprise, consist or consist essentially of administering concurrently to the subject a therapeutic agent and an HDAC7 polypeptide-producing agent as broadly described above and elsewhere herein, or a composition comprising a therapeutic agent and an HDAC7 polypeptide-producing agent as broadly described above and elsewhere herein.

In some embodiments, the inflammation is associated with the presence of an antigen-presenting cell that produces a pro-inflammatory mediator. For example, the pro-inflammatory mediator may be a pro-inflammatory cytokine. In specific embodiments, the pro-inflammatory cytokine is IL-1β or TNF.

In some embodiments of the disclosed method, the subject has or is at risk of developing an acute inflammatory condition. In other embodiments, the subject has or is at risk of developing a chronic inflammatory condition.

In another aspect of the present disclosure, methods are provided for treating, inhibiting or reducing, or slowing the progression of, an acute inflammatory condition in a subject. These methods generally comprises, consists or consists essentially of administering to the subject an effective amount of a therapeutic agent or composition as broadly described above and elsewhere herein.

In a related aspect, methods are disclosed for treating, preventing, inhibiting or reducing, or slowing the progression of, an acute inflammatory condition in a subject. These methods generally comprise, consist or consist essentially of administering concurrently to the subject a therapeutic agent and an HDAC7 polypeptide-producing agent as broadly described above and elsewhere herein, or a composition comprising a therapeutic agent and an HDAC7 polypeptide-producing agent as broadly described above and elsewhere herein.

Disclosed herein in another aspect, are methods for treating, inhibiting or reducing, or slowing the progression of, a chronic inflammatory condition in a subject. These methods generally comprise, consist or consist essentially of administering to the subject an effective amount of a therapeutic agent or composition as broadly described above and elsewhere herein.

Also disclosed in a related aspect are methods are disclosed for treating, preventing, inhibiting or reducing, or slowing the progression of, a chronic inflammatory condition in a subject. These methods generally comprise, consist or consist essentially of administering concurrently to the subject a therapeutic agent and an HDAC7 polypeptide-producing agent as broadly described above and elsewhere herein, or a composition comprising a therapeutic agent and an HDAC7 polypeptide-producing agent as broadly described above and elsewhere herein.

In related aspects, the present disclosure provides a use of a therapeutic agent or composition as broadly described above and elsewhere herein in the manufacture of a medicament for inhibiting or reducing pro-inflammatory activity of an antigen-presenting cell, or for treating, inhibiting or reducing, or slowing the progression of, inflammation, or for treating, inhibiting or reducing, or slowing the progression of, an acute inflammatory condition, or for treating, inhibiting or reducing, or slowing the progression of, a chronic inflammatory condition, in a subject.

The present disclosure also provides in related aspects a use of a therapeutic combination or composition comprising a therapeutic agent and an HDAC7 polypeptide-producing agent as broadly described above and elsewhere herein in the manufacture of a medicament for inhibiting or reducing pro-inflammatory activity of an antigen-presenting cell, or for treating, inhibiting or reducing, or slowing the progression of, inflammation, or for treating, inhibiting or reducing, or slowing the progression of, an acute inflammatory condition, or for treating, inhibiting or reducing, or slowing the progression of, a chronic inflammatory condition, in a subject.

### BRIEF DESCRIPTION OF THE DRAWINGS

FIG. 1 is a graphical representation showing the effect of reducing the expression of 6PGD by siRNA (Pgd siRNA) or reducing the activity of 6PGD by genetic targeting of HDAC7 (Hdac7^{-/-}) on the release of cytokines IL-1β and TNF. (A) The Pgd gene was silenced in mouse bone marrow-derived macrophages (BMM). Nigericin-mediated (i.e. inflammasome-triggered) IL-1β release from LPS-primed BMM was then assessed by ELISA. (B) BMM from HDAC7 knockout (Hdac7^{-/-}) or control (Hdac7^{+/+}) mice were infected with Escherichia coli (E. coli), then treated with Nigericin. Secreted IL-1β was assessed by ELISA. (C) The Pgd gene was silenced in BMM. LPS-induced TNF was assessed by ELISA. (D) HDAC7 knockout (Hdac7^{-/-}) or control (Hdac7^{+/+}) BMM were infected with E. coli. Secreted TNF was assessed by ELISA. All data were compiled from three independent experiments and are presented as mean±S.E.M. Statistical significance was determined using two-way ANOVA followed by Sidak's multiple comparison test (ns—not significant; * p<0.05; ** p<0.01)

FIG. 2 is a graphical representation showing the effect of over expression of the 6PGD gene on the release of cytokines IL-1β, TNF and IL-6. The Pgd gene was retrovirally overexpressed in BMM. Nigericin-triggered IL-1β release from LPS-primed cells (A), as well as LPS-induced TNF (B) and IL-6 (C), in culture supernatants were assessed by ELISA. Data were compiled from five independent experiments and are presented as mean±S.E.M. Statistical significance was determined using two-way ANOVA followed by Sidak's multiple comparison test (ns—not significant; * p<0.05; ** p<0.01).

FIG. 3 is a graphical representation showing the effect of R5LP treatment of macrophages on the release of cytokines IL-1β and TNF. (A-B) Human monocyte-derived macrophages (HMDM) were pre-treated with ribulose-5-phosphate (RL5P) for 16 h at the indicated concentrations. Nigericin-triggered IL-1β release from LPS-primed cells (A), as well as LPS-induced TNF (B) in culture supernatants were assessed by ELISA. (C-D) BMM were pre-treated for 16 h with RL5P at the indicated concentrations. Nigericin-triggered IL-1β release from LPS-primed cells (C), as well as LPS-induced TNF (D) in culture supernatants were assessed by ELISA. Data were compiled from six independent experiments (or donors) and are presented as mean±S.E.M. Statistical significance was determined using two-way ANOVA followed by Sidak's multiple comparison test (ns—not significant; * p<0.05; ** p<0.01).

FIG. 4 is a graphical representation showing the effect of R5LP treatment of macrophages on the release of cytokine IL-6. HMDM and BMM were pre-treated with the indicated concentrations of RL5P for 16 h, after which they were either stimulated with LPS (10 ng/mL) or infected with uropathogenic Escherichia coli (UPEC) strain EC958 (MOI 100) for 4 h. Supernatants were then assessed for secreted IL-6 by ELISA. Data were compiled from six independent experiments (or donors) and are presented as mean±SEM. Panel A presents IL-6 release (ng/mL), Panel B present IL-6 release normalized to LPS alone. Statistical significance was determined using repeated measure two-way ANOVA followed by Dunnett's multiple comparison test (A-B) (ns—not significant).

FIG. 5 is a graphical representation showing effect of increasing RL5P concentrations on the growth of uropathogenic Escherichia coli (UPEC) strain EC958 under conditions of oxidative stress in the form of 0.8 mM H₂O₂. (A) EC958 was cultured±H₂O₂ (0.8 mM) in the presence of the indicated concentrations of RL5P for 12 h, during which growth was assessed (A600 at the indicated time points); (B) Area under the curve analysis for the 12 h period was calculated thereafter and is presented in FIG. 5B. Data are compiled from four independent experiments and are presented as mean±SEM. Each symbol represents a different experiment. Statistical significance for (B) was determined using repeated measure two-way ANOVA followed by non-parametric Kruskal Wallis test followed by Dunn's multiple comparison test (ns—not significant; * p<0.05; **** p<0.0001).

FIG. 6 is a graphical representation showing effect of increasing RL5P concentrations on the growth of UPEC under conditions of oxidative stress in the form of 1 mM H₂O₂. (A) EC958 was cultured±H₂O₂ (1 mM) in the presence of the indicated concentrations of RL5P for 12 h, during which growth was assessed (A600 at the indicated time points); (B) EC958 was cultured in the presence of the indicated concentrations of RL5P and the absence of H₂O₂ for 12 h, during which growth was assessed (A600 at the indicated time points). Data are compiled from four independent experiments and are presented as mean±SEM.

FIG. 7 is a graphical representation showing the effect of RL5P and 6-phosphogluconate (6PG) on the on the growth of UPEC under conditions of oxidative stress in the form of H₂02. (A) EC958 was cultured±H₂O₂ (0.8 mM) in the presence of the indicated concentration of RL5P or 6PG for 12 h, during which growth was assessed (A600 at the indicated time points); (B). Area under the curve analysis for the 12 h period was calculated thereafter and is presented in FIG. 7B. Each symbol represents a different experiment. Statistical significance for (B) was determined using repeated measure two-way ANOVA followed by non-parametric Kruskal Wallis test followed by Dunn's multiple comparison test (ns—not significant; * p<0.05; **** p<0.0001).

FIG. 8 shows (A) a representative histogram plot showing the levels of GFP expression in the different transfection conditions, (B) median florescence intensity and (C) percentage of GFP positive cells are also shown (n=1 independent experiment). BMM were transfected with indicated amounts of GFP mRNA and lifted at 24 h post-transfection. GFP expression levels were then quantified using flow cytometry.

FIG. 9 is a graphical representation showing the mRNA levels of (A) Tnf, (B) Il6, (C) Il12b and (D) Ifnb1. BMM were transfected with GFP mRNA or treated with LPS (10 ng/mL, positive control) and RNA was extracted 4 h later. Levels of Tnf, Il6, Il12b and Ifnb1 mRNA were quantified using RT-qPCR and normalized to the housekeeping gene Hprt (n=1 independent experiment, error bars determined from technical triplicates, mean±SD).

FIG. 10 is A) a representative histogram plot showing the levels of GFP expression for different transfection conditions), (B) median fluorescence intensity, and (C) percentage of GFP positive cells are also shown (n=1 independent experiment). BMM were transfected with indicated amounts of unmodified (Unmod GFP) and 5-methoxyuridine modified GFP (Mod GFP) mRNAs, as well as a 5-methoxyuridine modified 6PGD mRNA (Mod PGD). Cells were then lifted at 24 h post-transfection. GFP expression levels were then quantified using flow cytometry.

FIG. 11 is a graphical representation showing the mRNA levels of (A) Tnf, (B) Il6 and (C) Ifnb1. BMM were transfected with indicated amounts of unmodified GFP mRNA (Unmod GFP), 5-methoxyuridine modified GFP mRNA (Mod GFP) or 5-methoxyuridine modified 6PGD mRNA (Mod PGD) or treated with LPS (10 ng/mL, positive control) and RNA was extracted 4 h later. mRNA levels of (A) Tnf, (B) 116 and (C) Ifnb1 are quantified using RT-qPCR and normalized to the housekeeping gene Hprt (n=1 independent experiment, error bars determined from technical triplicates, mean±SD).

FIG. 12 is (A) a representative histogram plot showing the levels of GFP expression in the different transfection conditions, (B) median florescence intensity, and (C) percentage of GFP positive cells are shown (n=1 independent experiment). BMM were transfected via nanoparticle delivery with indicated amounts of GFP mRNA and lifted at 24 h post-transfection. GFP expression levels were then quantified using flow cytometry.

FIG. 13 is a representative western blot showing levels of V5-tagged 6PGD and GAPDH (loading control) protein (n=2 independent experiments). BMM were transfected with indicated amounts of 5-methoxyuridine-modified 6PGD mRNA, unmodified GFP mRNA or 5-methoxyuridine modified GFP mRNA, then lysed at 24 h post-transfection. Levels of V5-tagged 6PGD and GAPDH protein were then assessed via western blot.

FIG. 14 is a graphical representation showing the mRNA levels of (A) Il1b, (B) Il6 and (C) Il12b, and the secreted protein levels of (D) IL-1β and (E) IL6. BMM were transfected with 500 ng of 5-methoxyuridine modified GFP mRNA (GFP), 5-methoxyuridine modified 6PGD mRNA (PGD) or vehicle (−) for 24 h. Cells were then treated±LPS (1 ng/mL) for 4 h. RNA was extracted and mRNA levels of (A) Il1b, (B) 116 and (C) Il12b were quantified using RT-qPCR and normalized to the housekeeping gene Hprt (n=3 independent experiments combined, normalized relative to transfected (−)+LPS for each experiment, mean±SEM). Statistical significance was determined using two-way ANOVA followed by Bonferroni multiple comparison test (ns, not significant; * p<0.05; ** p<0.01). Supernatants were collected, and protein levels quantified by ELISA as per the manufacturer's instructions (n=3 independent experiments combined, normalized relative to transfected (−)+LPS for each experiment, mean±SEM). Statistical significance was determined using two-way ANOVA followed by Bonferroni multiple comparison test (ns, not significant; * p<0.05).

FIG. 15 is a graphical representation showing the effect of nanoparticle delivery of 6PGD mRNA on body weight and liver Il1b mRNA levels in mice. (A) Weight loss in mice is shown as a percentage of starting body weight. Mice were injected with GFP or 6PGD mRNA formulated with silica-based nanoparticles (SNP) or lipid nanoparticles (LNP) overnight before injection with LPS (2 mg/kg) for 6 h (n=4 mice per group, individual data points+mean). (B) mRNA levels of Il1b in liver are shown. Livers were harvested from mice following injection with GFP or 6PGD mRNA formulated with silica-based nanoparticles (SNP) or lipid nanoparticles (LNP) overnight before injection with LPS (2 mg/kg). RNA was extracted and mRNA levels of Il1b were quantified using RT-qPCR and normalized to the housekeeping gene Hprt (n=4 mice per group, mean±SEM). Statistical significance was determined using two-way ANOVA followed by Bonferroni multiple comparison test (p-value displayed for non-significant data; * p<0.05).

FIG. 16 is a representative histology image of 6PGD protein expression in mice livers. Livers were harvested from mice following injection with (A) GFP- or 6PGD-encoding mRNA formulated with lipid nanoparticles (LNP) and (B) GFP- or 6PGD-encoding mRNA formulated with silica-based nanoparticles (SNP) overnight before injection with 2 mg/kg LPS. Livers were then fixed, embedded in paraffin and sectioned for histology analysis. Liver sections were examined by immunofluorescence for V5 expression (6PGD) and F4/80 expression (macrophage marker). DAPI was used to stain the nuclei. (A)→arrowheads indicate 6PDG positive cells and triangular arrowheads indicate F4/80 positive cells. (B)→arrowheads indicate 6PGD positive cells, triangular arrowheads indicate F4/80 positive cells and arrows indicate dual V5 and F4/80 positive cells. Images are representative of data from four mice per condition.

FIG. 17 is photographic representation showing a transmission electron microscopy (TEM) image of dendritic mesoporous organosilica nanoparticles (DMONs).

FIG. 18 is a graphical representation comparing Lipofectamine 2000 and DMONs as transfection agents for delivery and expression of GFP mRNA in RAW264.7 cells after 48 h. (A) percentage of GFP positive M0, M1 or M2 polarized cells after transfection with GFP mRNA, GFP mRNA+lipofectamine or GFP mRNA packaged in DMONs. (B) mean fluorescent intensity of transfected M0, M1 or M2 polarized cells after transfection with GFP mRNA, GFP mRNA+lipofectamine or GFP mRNA packaged in DMONs. (C) Fold change in transfection efficacy for chloroquine loaded DMONs-40-R0.2-REI compared to without loading. (D) Mean fluorescent intensity of GFP mRNA delivered by SNPs-PEI-PEG compared to Lipofectamine MessengerMax.

FIG. 19 is a graphical representation of HDAC7 increasing 6PGD enzyme activity in HEK293 cells. HEK293 cells were transfected with expression constructs encoding (A-B) 6PGD and/or HDAC7, (C-D) 6PGD and HDAC7 wild type (WT) or HDAC7 enzyme-dead (ED), (E-F) 6PGD and full-length (HDAC7), N-terminal HDAC7 (N-HDAC7) or C-terminal HDAC7 (C-HDAC7) at a molar ratio of 1:1 (6PGD:HDAC). After 24 h, cells were lysed and the enzymatic activity of 6PGD in lysates was measured. Data (mean±SEM (A) or mean±SD (C, E), n=3) are displayed as relative activity by normalizing to enzyme activity in cells transfected with 6PGD alone. Statistical analysis was performed using repeated measure one-way ANOVA followed by Dunnett's multiple comparison (ns, not significant; * p<0.05; ** p<0.01). (B, D, F) Levels of 6PGD (Flag), HDAC7 (V5) and GAPDH expression in cell lysates were assessed via western blot.

### DETAILED DESCRIPTION

### 1. Definitions

Unless defined otherwise, all technical and scientific terms used herein have the same meaning as commonly understood by those of ordinary skill in the art to which the disclosure belongs. Although any methods and materials similar or equivalent to those described herein can be used in the practice or testing of the present disclosure, preferred methods and materials are described. For the purposes of the present disclosure, the following terms are defined below.

The articles “a” and “an” are used herein to refer to one or to more than one (i.e., to at least one) of the grammatical object of the article. By way of example, reference to “an agent” includes a single agent, as well as two or more agents; reference to “the disclosure” includes single and multiple aspects taught by the disclosure; and so forth.

Further, the term “about”, as used herein when referring to a measurable value such as an amount, dose, time, temperature, activity, level, number, frequency, percentage, dimension, size, amount, weight, position, length and the like, is meant to encompass variations of ±15%, ±10%, ±5%, ±1%, ±0.5%, or even ±0.1% of the specified amount, dose, time, temperature, activity, level, number, frequency, percentage, dimension, size, amount, weight, position, length and the like.

Where a range of values is provided, it is understood that each intervening value, to the tenth of the unit of the lower limit unless the context clearly dictates otherwise, between the upper and lower limit of that range and any other stated or intervening value in that stated range is encompassed within the disclosure. The upper and lower limits of these smaller ranges may independently be included in the smaller ranges is also encompassed within the disclosure, subject to any specifically excluded limit in the stated range. Where the stated range includes one or both of the limits, ranges excluding either both of those included limits are also included in the disclosure.

The term “acute inflammatory condition” as used herein refers to a condition in which acute inflammation is present and represents a rapid, short-lived (minutes to days), relatively uniform response to acute injury characterized by accumulations of fluid, plasma proteins, and neutrophilic leukocytes. In acute inflammation, removal of the stimulus halts the recruitment of monocytes (which become macrophages under appropriate activation) into the inflamed tissue, and existing macrophages exit the tissue via lymphatics. Examples of injurious agents that cause acute inflammation include, but are not limited to, pathogens (e.g., bacteria, viruses, parasites), foreign bodies from exogenous (e.g., asbestos) or endogenous (e.g., urate crystals, immune complexes), sources, and physical (e.g., burns) or chemical (e.g., caustics) agents. Generally, the physiologic changes accompanying acute inflammation encompass four main features: (1) vasodilation, which results in a net increase in blood flow, is one of the earliest s physical responses to acute tissue injury; (2) in response to inflammatory stimuli, endothelial cells lining the venules upregulate cell surface proteins (e.g., E-selectin) enabling leukocyte binding and diapedesis and also contract, widening the intracellular junctions to produce gaps, leading to increased vascular permeability, which permits leakage of plasma proteins and blood cells out of blood vessels; (3) inflammation often is characterized by a strong infiltration of leukocytes at the site of inflammation, particularly neutrophils (polymorphonuclear cells). These cells promote tissue damage by releasing toxic substances at the vascular wall or in uninjured tissue; and (4) fever, produced by pyrogens released from leukocytes in response to specific stimuli. Other conditions include a reduction in olfactory sensation and adverse effects on the cardiovascular system.

The terms “administration concurrently” or “administering concurrently” or “co-administering” and the like refer to the administration of a single composition containing two or more actives, or the administration of each active as separate compositions and/or delivered by separate routes either contemporaneously or simultaneously or sequentially within a short enough period of time that the effective result is equivalent to that obtained when all such actives are administered as a single composition. By “simultaneously” is meant that the active agents are administered at substantially the same time, and desirably together in the same formulation. By “contemporaneously” it is meant that the active agents are administered closely in time, e.g., one agent is administered within from about one minute to within about one day before or after another. Any contemporaneous time is useful. However, it will often be the case that when not administered simultaneously, the agents will be administered within about one minute to within about eight hours and suitably within less than about one to about four hours. When administered contemporaneously, the agents are suitably administered at the same site on the subject. The term “same site” includes the exact location, but can be within about 0.5 to about 15 centimeters, preferably from within about 0.5 to about 5 centimeters. The term “separately” as used herein means that the agents are administered at an interval, for example at an interval of about a day to several weeks or months. The active agents may be administered in either order. The term “sequentially” as used herein means that the agents are administered in sequence, for example at an interval or intervals of minutes, hours, days or weeks. If appropriate the active agents may be administered in a regular repeating cycle.

As used herein, and in particular with respect to any active agent or molecule (i.e., an RL5P analog), the term “analog” is a structural and/or functional analog of said active agent or molecule. A structural analog may be any compound having a structure similar to that of the active agent or molecule, but differ in one or more atoms, functional groups, or substructures or the like. Functional analogs are compounds with similar physical, chemical, biochemical, or pharmacological properties to the active agent or molecule. Not all structural analogs are functional analogs and not all functional analogs are structural analogs. However, it can be envisioned that a structural analog of, for example, 6PGD, could be converted by an RL5P-producing agent to produce RL5P or a functional RL5P analog. In some embodiments of the present disclosure, the RL5P analog is a functional analog of RL5P.

As used herein, “and/or” refers to and encompasses any and all possible combinations of one or more of the associated listed items, as well as the lack of combinations when interpreted in the alternative (or).

As used herein, the term “antigen-presenting cell” (APC) is a cell that displays antigen in the context of major histocompatibility complex (MHC) on its surface. T cells may recognize this complex using their T cell receptor (TCR). Antigen-presenting cells process antigens and present them to T cells. An antigen presenting cell includes, but is not limited to, monocytes/macrophages, B cells and dendritic cells (DCs). According to the disclosure, the term “antigen-presenting cell” includes professional antigen-presenting cells and non-professional antigen-presenting cells. The term “professional antigen presenting cells” relates to antigen presenting cells which constitutively express the Major Histocompatibility Complex class II (MHC class II) molecules required for interaction with T cells. Professional antigen-presenting cells are very efficient at internalizing antigen, either by phagocytosis or by receptor-mediated endocytosis, and then displaying a fragment of the antigen, bound to a class II MHC molecule, on their membrane. The T cell recognizes and interacts with the antigen-class II MHC molecule complex on the membrane of the antigen-presenting cell. An additional co-stimulatory signal is then produced by the antigen-presenting cell, leading to activation of the T cell. The expression of co-stimulatory molecules is a defining feature of professional antigen-presenting cells. The main types of professional antigen-presenting cells are dendritic cells, macrophages, B-cells, and certain activated epithelial cells. The term “non-professional antigen presenting cells” relates to antigen presenting cells which do not constitutively express MHC class II molecules, but upon stimulation by certain cytokines such as interferon-gamma. Exemplary, non-professional antigen presenting cells include fibroblasts, thymic epithelial cells, thyroid epithelial cells, glial cells, pancreatic beta cells or vascular endothelial cells.

As used herein, the term “assembly” refers to a plurality of interconnected molecules, including a plurality of interconnected polymer chains. In some cases, the polymer chains may be interconnected via bonds, including, for example, covalent bonds (e.g., carbon-carbon, carbon-oxygen, oxygen-silicon, sulfur-sulfur, phosphorus-nitrogen, carbon-nitrogen, metal-oxygen or other covalent bonds), ionic bonds, hydrogen bonds (e.g., between hydroxyl, amine, carboxyl, thiol and/or similar functional groups, for example), dative bonds (e.g., complexation or chelation between metal ions and monodentate or multidentate ligands), or the like. The interaction may also comprise, in some instances, Van der Waals interactions or a binding event between pairs of molecules, such as biological molecules, for example. In some embodiments, the assembly includes particles such as nanoparticles and microparticles.

As used herein, the term “associated with”, in the context of the agents of the present disclosure (e.g., therapeutic agents), refers to the state of two or more entities that are linked by a direct or indirect covalent or non-covalent interaction. In some embodiments, an association is covalent. In some embodiments, a covalent association is mediated by a linker moiety. In some embodiments, an association is non-covalent (e.g., charge interactions, affinity interactions, metal coordination, physical adsorption, host-guest interactions, hydrophobic interactions, n-n stacking interactions, hydrogen bonding interactions, van der Waals interactions, magnetic interactions, electrostatic interactions, dipole-dipole interactions, etc.). For example, in some embodiments, an entity (e.g., a cargo to be delivered such as RL5P, a RL5P analog, a RL5P-analog-producing agent and/or a RL5P-producing agent) may be covalently associated with a polymer chain or assembly comprising a plurality of polymer chains. In some embodiments, an entity (e.g., a cargo to be delivered such as RL5P, a RL5P analog, a RL5P-analog-producing agent and/or a RL5P-producing agent) may be non-covalently associated with a polymer chain or assembly comprising a plurality of polymer chains, (e.g., the entity may be associated with the surface of, encapsulated within, surrounded by, and/or distributed throughout an assembly comprising a plurality of polymer chains). In specific embodiments, an entity (e.g., RL5P, a RL5P analog, a RL5P-analog-producing agent and/or a RL5P-producing agent) may be covalently associated with a targeting moiety, while in other embodiments, an entity (e.g., RL5P, a RL5P analog, a RL5P-analog-producing agent and/or a RL5P-producing agent) may be non-covalently associated with a targeting moiety. Alternatively, in some embodiments, an entity (e.g., RL5P, a RL5P analog, a RL5P-analog-producing agent and/or a RL5P-producing agent) may be non-covalently associated with a polymer chain or assembly comprising a plurality of polymer chains (e.g., the components of a particle), while at the same time, the polymer chain or assembly comprising a plurality of polymer chains may be covalently associated with a targeting moiety.

As used herein, the term “cargo” refers to a biologically active molecule (e.g., an anti-inflammatory agent) that acts on a target (e.g., a target cell, including an immune cell such as an APC) that is associated with and/or encapsulated by a particle (or delivery vehicle) or targeting moiety of the present disclosure. Non-limiting examples of cargos that can be introduced into an immune cell such as an APC include molecules such as, nucleic acids (e.g., DNA or mRNA molecules that encode a polypeptide such as an enzyme (e.g., 6PGD, HDAC7, or both), lipids, carbohydrates (e.g., RL5P or RL5P analog), and small molecules (e.g., small molecule drugs).

As used herein, the term “chronic inflammatory condition” refers to a condition characterized by a persistent inflammatory response with pathologic sequelae. This state is characterized by infiltration of mononuclear cells, proliferation of fibroblasts and small blood vessels, increased connective tissue, and tissue destruction. Examples of chronic inflammatory diseases include, but are not limited to, Crohn's disease, psoriasis, chronic obstructive pulmonary disease, inflammatory bowel disease, multiple sclerosis, chronic liver disease, and asthma. Autoimmune diseases such as rheumatoid arthritis and systemic lupus erythematosus can also result in a chronic inflammatory state.

The term “coding sequence” or “coding region”, as used herein, refers to a polynucleotide sequence, which specifies the amino acid sequence of a polypeptide or portion of the polypeptide. The boundaries of a coding sequence for a polypeptide are generally determined by an open reading frame, which usually begins with the ATG start codon or alternative start codons such as GTG and TTG and ends with a stop codon such as TAA, TAG, and TGA. The coding sequence may be a sequence of genomic DNA, cDNA, a synthetic polynucleotide, and/or a recombinant polynucleotide.

Throughout this specification, unless the context requires otherwise, the words “comprise,” “comprises” and “comprising” will be understood to imply the inclusion of a stated step or element or group of steps or elements but not the exclusion of any other step or element or group of steps or elements. Thus, use of the term “comprising” and the like indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

A “conservative amino acid substitution” is one in which the amino acid residue is replaced with an amino acid residue having a similar side chain. Families of amino acid residues having similar side chains have been defined in the art, which can be generally sub-classified as follows:

TABLE A
AMINO ACID SUB-CLASSIFICATION

| Sub-classes | Amino acids |
|---|---|
| Acidic | Aspartic acid, Glutamic acid |
| Basic | Noncyclic: Arginine, Lysine; Cyclic: Histidine |
| Charged | Aspartic acid, Glutamic acid, Arginine, Lysine, |
| | Histidine |
| Small | Glycine, Serine, Alanine, Threonine, Proline |
| Polar/neutral | Asparagine, Histidine, Glutamine, Cysteine, Serine, |
| | Threonine |
| Polar/large | Asparagine, Glutamine |
| Hydrophobic | Tyrosine, Valine, Isoleucine, Leucine, Methionine, |
| | Phenylalanine, Tryptophan |
| Aromatic | Tryptophan, Tyrosine, Phenylalanine |
| Residues that | Glycine and Proline |
| influence chain | |
| orientation | |

Conservative amino acid substitution also includes groupings based on side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. For example, it is reasonable to expect that replacement of a leucine with an isoleucine or valine, an aspartate with a glutamate, a threonine with a serine, or a similar replacement of an amino acid with a structurally related amino acid will not have a major effect on the properties of the resulting variant polypeptide. Whether an amino acid change results in a functional polypeptide can readily be determined by assaying its activity. Conservative substitutions are shown in Table 2 under the heading of exemplary and preferred substitutions. Amino acid substitutions falling within the scope of the disclosure, are, in general, accomplished by selecting substitutions that do not differ significantly in their effect on maintaining (a) the structure of the peptide backbone in the area of the substitution, (b) the charge or hydrophobicity of the molecule at the target site, or (c) the bulk of the side chain. After the substitutions are introduced, the variants are screened for biological activity.

TABLE B
EXEMPLARY AND PREFERRED
AMINO ACID SUBSTITUTIONS

| Original | | |
|---|---|---|
| Residue | Exemplary Substitutions | Preferred Substitutions |
| Ala | Val, Leu, Ile | Val |
| Arg | Lys, Gln, Asn | Lys |
| Asn | Gln, His, Lys, Arg | Gln |
| Asp | Glu | Glu |
| Cys | Ser | Ser |
| Gln | Asn, His, Lys, | Asn |
| Glu | Asp, Lys | Asp |
| Gly | Pro | Pro |
| His | Asn, Gln, Lys, Arg | Arg |
| Ile | Leu, Val, Met, Ala, Phe, Norleu | Leu |
| Leu | Norleu, Ile, Val, Met, Ala, Phe | Ile |
| Lys | Arg, Gln, Asn | Arg |
| Met | Leu, Ile, Phe | Leu |
| Phe | Leu, Val, Ile, Ala | Leu |
| Pro | Gly | Gly |
| Ser | Thr | Thr |
| Thr | Ser | Ser |
| Trp | Tyr | Tyr |
| Tyr | Trp, Phe, Thr, Ser | Phe |
| Val | Ile, Leu, Met, Phe, Ala, Norleu | Leu |

As used herein, the term “contacting” in the context of contacting a cell with a therapeutic agent of the present disclosure means bringing a therapeutic agent into contact with the cell, or vice-versa, or any other manner of causing the therapeutic agent and the cell to come into contact. In those embodiments of the method for inhibiting or reducing pro-inflammatory activity of an antigen-presenting cell, or for treating, inhibiting or reducing, or slowing the progression of, inflammation in a subject, the therapeutic agent is administered to a subject, and the administration may occur by any route (e.g., topical, oral, parenteral, subcutaneous, transdermal, transbuccal, intravascular (e.g., intravenous or intra-arterial), intramuscular, subcutaneous, intranasal, and intra-ocular administration).

The terms “corresponds to” and “corresponding to” and their grammatical equivalents as applied to nucleic acid sequences refer to a nucleic acid sequence that displays substantial sequence identity to a reference nucleic acid sequence (e.g., at least about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 97, 88, 89, 90, 91, 92, 93, 94, 95, 96, 97, 98, 99% or even up to 100% sequence identity to all or a portion of the reference nucleic acid sequence). In the context of amino acid sequence, the terms “corresponds to” and “corresponding to” and their grammatical equivalents refer to an amino acid sequence that displays substantial sequence similarity or identity to a reference amino acid sequence. In general the amino acid sequence will display at least about 70, 71, 72, 73, 74, 75, 76, 77, 78, 79, 80, 81, 82, 83, 84, 85, 86, 97, 88, 89, 90, 91, 92, 93, 94, 95, 96, 97, 98, 99% or even up to 100% sequence similarity or identity to at least a portion of the reference amino acid sequence.

As used herein, “delaying progression of a condition” or “decreasing the rate of progression of a condition” means to defer, hinder, slow, retard, stabilize, and/or postpone development of the condition. This delay can be of varying lengths of time, depending on the history of the condition and/or individual being treated.

By “effective amount”, in the context of treating or preventing a condition is meant the administration of an amount of an agent or composition to an individual in need of such treatment or prophylaxis, either in a single dose or as part of a series, that is effective for the prevention of incurring a symptom, holding in check such symptoms, and/or treating existing symptoms, of that condition. The effective amount will vary depending upon the health and physical condition of the individual to be treated, the taxonomic group of individual to be treated, the formulation of the composition, the assessment of the medical situation, and other relevant factors. It is expected that the amount will fall in a relatively broad range that can be determined through routine trials. Non-limiting symptoms of inflammation include, for example, redness, pain, heat, fluid retention, fatigue, loss of function, headaches, loss of appetite, tenderness, pain, muscle stiffness, scarring and hyperplasia.

As used herein the term “enhancing the activity of 6PGD” means causing a significant increase (suitably a statistically significant increase) in the enzymatic activity, preferably phosphogluconate dehydrogenase, of 6PGD (e.g., an increase of at least about 10%, at least about 20%, at least about 25%, at least about 33%, at least about 50%, at least about 100%, at least about 2 fold, at least about, 3 fold at least, about 10 fold, at least about 100 fold or more).

The term “expression” with respect to a gene sequence refers to transcription of the gene to produce a RNA transcript (e.g., mRNA, antisense RNA, siRNA, shRNA, miRNA, etc.) and, as appropriate, translation of a resulting mRNA transcript to a protein. Thus, as will be clear from the context, expression of a coding sequence results from transcription and translation of the coding sequence. Conversely, expression of a non-coding sequence results from the transcription of the non-coding sequence.

The terms “histone deacetylase 7”, “histone deacetylase 7 polypeptide”, “HDAC7” and “HDAC7 polypeptide” are used interchangeably herein to refer to a polypeptide having an amino acid sequence corresponding to a naturally-occurring HDAC7 polypeptide. This term encompasses, without limitation, polypeptides having an amino acid sequence that shares at least 80% (and at least 81% to at least 99% and all integer percentages in between) sequence identity or similarity with the sequence set forth in any one of SEQ ID NOs: 12-15 and that enhances the activity of 6PGD. In some embodiments, the HDAC7 polypeptide comprises, consists or consists essentially of a PHA03247 super family motif (National Center for Biotechnology Information (NCBI) Accession No. c133720; Position-Specific Score Matrix (PPSM) Id: 223021; Lu et al. “CDD/SPARCLE: the conserved domain database in 2020.”, Nucleic Acids Res. 2020; 48(D1):D265-D268) corresponding to the herpes simplex virus-1 UL36 large tegument protein, which suitably comprises deubiquitinating activity (Schlieker et al., J Virol. 2005; 79(24):15582-5). It further encompasses natural allelic variation of HDAC7 polypeptides, including isoforms, that may exist within a species or occur from one organism to another. Also, degree and location of glycosylation or other post-translation modifications may vary depending on the chosen host and the nature of the hosts cellular environment. The term “HDAC7 polypeptide” is also intended to encompass HDAC7 polypeptides that have been processed to yield their respective bioactive forms. It further encompasses HDAC7 polypeptides that have either been chemically modified relative to a reference or naturally-occurring HDAC7 polypeptide and/or contain one or more amino acid sequence alterations relative to a reference or naturally-occurring HDAC7 polypeptide and/or contain truncated amino acid sequences relative to a reference or naturally-occurring full-length or precursor HDAC7 polypeptide or domains thereof, including a signal peptide, for example, that is removed in the mature form. The term “HDAC7 polypeptide” also encompasses proteinaceous molecules with a slightly modified amino acid sequence, for instance, polypeptides that have been chemically modified relative to a reference or naturally-occurring neuritin polypeptide. HDAC7 polypeptides also include, without limitation, polypeptides having an amino acid sequence that differs from the sequence of a reference or naturally-occurring HDAC7 polypeptide by insertion, deletion, or substitution of one or more amino acids and in illustrative examples, encompass proteinaceous molecules that exhibit at least about 10%, 20%, 30%, 40%, 50%, 60%, 70%, 80%, 90%, 100%, 110%, 120%, and 130% of the 6PGD-enhancing activity of a reference or naturally-occurring HDAC7 polypeptide that has been produced in the same cell or cell type.

As used herein, the term “immune cell” refers to any cell that plays a role in the immune response of a subject. Representative immune cells elicits a pro-inflammatory immune response, including, for example, the production of pro-inflammatory mediators. Illustrative immune cells include immune cells of hematopoietic origin including: lymphocytes, such as B cells and T cells; natural killer cells; myeloid cells, such as monocytes, macrophages, dendritic cells, eosinophils, mast cells, basophils, and granulocytes. In specific embodiments, the immune cell is an antigen-presenting cell. In certain embodiments, the immune cell is other than a T cell.

The term “inflammation” refers to an organism's (e.g., a mammal's) coordinated response to harmful stimuli such as pathogens, damaged cells (e.g., wounds), irritants and toxic substances, with the immune system playing a central role in this process. The contribution of the immune system to inflammation can involve innate immune components and/or adaptive immunity. Inflammation is generally characterized as either chronic or acute. Acute inflammation can be characterized by, as non-limiting examples, redness, pain, heat, swelling, and/or loss of function due to infiltration of plasma proteins and leukocytes to the affected area. Acute inflammation is associated with acute inflammatory conditions. Chronic inflammation can be characterized by, as non-limiting examples, persistent inflammation, tissue destruction, and/or attempts at repair. Monocytes, macrophages, plasma B cells, and other lymphocytes are commonly recruited to the affected area, and angiogenesis and fibrosis can occur, in some instances leading to scar tissue. Chronic inflammation is associated with chronic inflammatory conditions. The term “inflammatory response” refers to the specific mechanisms by which inflammation is achieved and regulated, including, merely by way of illustration, immune cell activation or migration, cytokine production, vasodilation, including kinin release, fibrinolysis, and coagulation, among others described herein and known in the art.

The term “macrophage” refers to a subgroup of phagocytic cells produced either by the differentiation of monocytes or from cells seeded in specific tissues during embryogenesis. Macrophages which are activated by inflammation, immune cytokines or microbial products nonspecifically engulf and kill foreign pathogens within the macrophage by hydrolytic and oxidative attack resulting in degradation of the pathogen. Peptides from degraded proteins are displayed on the macrophage cell surface where they can be recognized by immune cells, such as T cells, resulting in the immune response. Macrophages belong to the class of antigen presenting cells.

As used herein, “microparticle” refers to a particle having at least one dimension in the range of about 1 μm to about 100 μm, including any integer value between about 1 μm and about 1000 μm (including all integers and fractional integers in between). Exemplary microparticles have a diameter of less than about 100 microns, less than about 50 microns, less than about 10 microns, less than about 5 microns, or less than about 3 microns, or less than about 2 microns. The particles can have any shape. Microparticles having a spherical shape are generally referred to as “microspheres”.

As used herein, the term “nanoparticle,” refers to a particle having at least one dimension in the range of about 1 nm to about 1000 nm, including any integer value between 1 nm and 1000 nm (including about 1, 2, 5, 10, 20, 50, 60, 70, 80, 90, 100, 200, 500, and 1000 nm and all integers and fractional integers in between). In some embodiments, the nanoparticle has at least one dimension, e.g., a diameter, of about 100 nm. In some embodiments, the nanoparticle has a diameter of about 200 nm. In other embodiments, the nanoparticle has a diameter of about 500 nm. In yet other embodiments, the nanoparticle has a diameter of about 1000 nm (1 μm). In such embodiments, the particle also can be referred to as a “microparticle.”

The term “operably connected” or “operably linked” as used herein refers to a juxtaposition wherein the components so described are in a relationship permitting them to function in their intended manner. For example, a regulatory sequence (e.g., a promoter) “operably linked” to a nucleotide sequence of interest (e.g., a coding and/or non-coding sequence) refers to positioning and/or orientation of the control sequence relative to the nucleotide sequence of interest to permit expression of that sequence under conditions compatible with the control sequence. The control sequences need not be contiguous with the nucleotide sequence of interest, so long as they function to direct its expression. Thus, for example, intervening non-coding sequences (e.g., untranslated, yet transcribed, sequences) can be present between a promoter and a coding sequence, and the promoter sequence can still be considered “operably linked” to the coding sequence.

The term “particle” refers to a small object, fragment, or piece of a substance that may be a inorganic material, organic material, or mixture thereof, typically with a diameter of less than 1000 microns, which is suitably sized for delivery of the agents disclosed herein to immune cells such as APCs. Typically, the particle has an average characteristic dimension of about less than about 1 mm and greater than at least 1 nm, where the characteristic dimension, or “critical dimension,” of the particle is the smallest cross-sectional dimension of the particle. A particle may be composed of a single substance or multiple substances. The term “particle” as used herein encompasses microparticles, nanoparticles, and picoparticles. In some embodiments, particles can be a polymeric particle, non-polymeric particle (e.g., a metal particle, quantum dot, ceramic, inorganic particles, inorganic particles coated with polymer or lipid, carbon nanoassemblies, paramagnetic particles, ferromagnetic particles, etc.), liposomes, lipid-based particles, lipid carriers such as lipidoids, lipoplexes, polymeric particles, micelles, filomicelles, exosomes, peptide carriers, lipoproteins, lipid-coated bubbles, polymersomes, niosomes, nanotubes, microvesicles, dendrimers, hyperbranched polymers and conjugates, hybrids thereof, and/or combinations thereof.

The term “particle size” (or “nanoparticle size” or “microparticle size”) as used herein refers to the median size in a distribution of particles, nanoparticles or microparticles. The median size is determined from the average linear dimension of individual nanoparticles, for example, the diameter of a spherical nanoparticle. Size may be determined by any number of methods in the art, including dynamic light scattering (DLS) and transmission electron microscopy (TEM) techniques.

The terms “patient”, “subject”, “host” or “individual” used interchangeably herein, refer to any subject, particularly a vertebrate subject, and even more particularly a mammalian subject, for whom therapy or prophylaxis is desired. Suitable vertebrate animals that fall within the scope of the disclosure include, but are not restricted to, any member of the subphylum Chordata including primates (e.g., humans, monkeys and apes, and includes species of monkeys such from the genus Macaca (e.g., cynomolgus monkeys such as Macaca fascicularis, and/or rhesus monkeys (Macaca mulatta)) and baboon (Papio ursinus), as well as marmosets (species from the genus Callithrix), squirrel monkeys (species from the genus Saimiri) and tamarins (species from the genus Saguinus), as well as species of apes such as chimpanzees (Pan troglodytes)), rodents (e.g., mice rats, guinea pigs), lagomorphs (e.g., rabbits, hares), bovines (e.g., cattle), ovines (e.g., sheep), caprines (e.g., goats), porcines (e.g., pigs), equines (e.g., horses), canines (e.g., dogs), felines (e.g., cats), avians (e.g., chickens, turkeys, ducks, geese, companion birds such as canaries, budgerigars etc.), marine mammals (e.g., dolphins, whales), reptiles (snakes, frogs, lizards etc.), and fish. In specific embodiments, the subject is a primate such as a human.

By “pharmaceutically acceptable carrier” is meant a pharmaceutical vehicle comprised of a material that is not biologically or otherwise undesirable, i.e., the material may be administered to a subject along with the selected active agent without causing any or a substantial adverse reaction. Carriers may include excipients and other additives such as diluents, fillers, detergents, coloring agents, wetting or emulsifying agents, pH buffering agents, preservatives and the like.

A “polyA tail” is a region of mRNA that is downstream, e.g., directly downstream (i.e., 3′), from the 3′ UTR that contains multiple, consecutive adenosine monophosphates. A polyA tail may contain 10 to 300 adenosine monophosphates. For example, a polyA tail may contain 10, 20, 30.40, 50, 60, 70, 80, 90, 100, 110, 120, 130, 140, 150, 160, 170, 180, 190, 200, 210, 220, 230, 240, 250, 260, 270, 280, 290 or 300 adenosine monophosphates. In some embodiments, a polyA tail contains 50 to 250 adenosine monophosphates. In a relevant biological setting (e.g., in cells, in vivo) the poly(A) tail functions to protect mRNA from enzymatic degradation, e.g., in the cytoplasm, and aids in transcription termination, export of the mRNA from the nucleus and translation.

The term “polynucleotide” or “nucleic acid” as used herein designates mRNA, RNA, cRNA, cDNA or DNA. The term typically refers to polymeric forms of nucleotides of at least 10 bases in length, either ribonucleotides or deoxynucleotides or a modified form of either type of nucleotide. The term includes single and double stranded forms of DNA.

“Polypeptide”, “peptide”, “protein” and “proteinaceous molecule” are used interchangeably herein to refer to molecules comprising or consisting of a polymer of amino acid residues and to variants and synthetic analogs of the same. Thus, these terms apply to amino acid polymers in which one or more amino acid residues are synthetic non-naturally occurring amino acids, such as a chemical analog of a corresponding naturally occurring amino acid, as well as to naturally-occurring amino acid polymers.

The term “pro-inflammatory mediator” means an immunoregulatory agent that favors inflammation. Such agents include, cytokines such as chemokines, interleukins (IL), lymphokines, and tumor necrosis factor (TNF) as well as growth factors and oxidative stressors. In specific embodiments, the pro-inflammatory mediator is a “pro-inflammatory cytokine”. Typically, pro-inflammatory cytokines include IL-1α, IL-1β, IL-6, and TNF (also known as TNF-α), which are largely responsible for early responses. Other pro-inflammatory mediators include, but are not limited to, LIF, IFN-γ, IFN-β, IFN-α, OSM, CNTF, TGF-.β, GM-CSF, TWEAK, IL-11, IL-12, IL-15, IL-17, IL-18, IL-19, IL-20, IL-8, IL-16, IL-22, IL-23, IL-31, IL-32 and IL-33 (Tato et al., 2008. Cell 132:900; Cell 132:500, Cell 132:324) and chemokines such as CCL2, CCL3 and CXCL10. Pro-inflammatory mediators may act as endogenous pyrogens (IL-1, IL-6, IL-17, TNF), up-regulate the synthesis of secondary mediators and pro-inflammatory cytokines by both macrophages and mesenchymal cells (including fibroblasts, epithelial and endothelial cells), stimulate the production of acute phase proteins, attract inflammatory cells, cause aberrant wound healing/scarring and initiate cell death and tissue destruction. In specific embodiments, the term “pro-inflammatory cytokine” relates to pro-inflammatory cytokines other than IL-6 and in preferred embodiments, the pro-inflammatory cytokine is selected from TNF and IL-1β.

“Regulatory elements”, “regulatory sequences”, control elements”, “control sequences” and the like are used interchangeably herein to refer to nucleotide sequences located upstream (5′ non-coding sequences), within, or downstream (3′ non-coding sequences) of a coding sequence, and which influence the transcription, RNA processing or stability, or translation of the associated coding sequence, either directly or indirectly. Regulatory elements include transcriptional enhancers, anchor sequences, anchor sequence-mediated conjunctions, promoters, transcriptional repressors, translation leader sequences, introns, Rep recognition elements, intergenic regions and polyadenylation signal sequences. They include natural and synthetic sequences as well as sequences which may be a combination of synthetic and natural sequences.

The term “sequence identity” as used herein refers to the extent that sequences are identical on a nucleotide-by-nucleotide basis or an amino acid-by-amino acid basis over a window of comparison. Thus, a “percentage of sequence identity” is calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity.

“Similarity” refers to the percentage number of amino acids that are identical or constitute conservative substitutions as defined in Tables A and B supra. Similarity may be determined using sequence comparison programs such as GAP (Deveraux et al. 1984, Nucleic Acids Research 12: 387-395). In this way, sequences of a similar or substantially different length to those cited herein might be compared by insertion of gaps into the alignment, such gaps being determined, for example, by the comparison algorithm used by GAP.

Terms used to describe sequence relationships between two or more polynucleotides or polypeptides include “reference sequence”, “comparison window”, “sequence identity”, “percentage of sequence identity” and “substantial identity”. A “reference sequence” can be at least 12 but frequently 14 to 18 and often at least 25 monomer units, inclusive of nucleotides and amino acid residues, in length. In certain embodiments, “reference sequence” is at least 14 and up to 29 (e.g., 14, 15, 16, 17, 18, 19, 20, 21, 22, 23, 24, 25, 26, 27, 28, 29) nucleobases. Because two nucleic acids may each comprise (1) a sequence that is similar between the two nucleic acids, and (2) a sequence that is divergent between the two nucleic acids, sequence comparisons between two (or more) nucleic acids are typically performed by comparing sequences of the two nucleic acids over a “comparison window” to identify and compare local regions of sequence similarity. A “comparison window” refers to a conceptual segment of at least 6 contiguous positions, usually about 50 to about 100, more usually about 100 to about 150 in which a sequence is compared to a reference sequence of the same number of contiguous positions after the two sequences are optimally aligned. The comparison window may comprise additions or deletions (i.e., gaps) of about 20% or less as compared to the reference sequence (which does not comprise additions or deletions) for optimal alignment of the two sequences. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, WI, USA) or by inspection and the best alignment (i.e., resulting in the highest percentage homology over the comparison window) generated by any of the various methods selected. Reference also may be made to the BLAST family of programs as for example disclosed by Altschul et al., 1997, Nucl. Acids Res. 25:3389. A detailed discussion of sequence analysis can be found in Unit 19.3 of Ausubel et al., “Current Protocols in Molecular Biology”, John Wiley & Sons Inc, 1994-1998, Chapter 15.

“Stringency” as used herein refers to the temperature and ionic strength conditions, and presence or absence of certain organic solvents, during hybridization. The higher the stringency, the higher will be the observed degree of complementarity between sequences. “Stringent conditions” as used herein refers to temperature and ionic conditions under which only polynucleotides having a high proportion of complementary nucleobases, preferably having exact complementarity, will hybridize. The stringency required is nucleotide sequence dependent and depends upon the various components present during hybridization and is greatly changed when nucleotide analogs are used. Generally, stringent conditions are selected to be about 10° C. to 20° C. less than the thermal melting point for the specific sequence at a defined ionic strength and pH. The Tm is the temperature (under defined ionic strength and pH) at which 50% of a target sequence hybridizes to a complementary probe. It will be understood that a polynucleotide will hybridize to a target sequence under at least low stringency conditions, preferably under at least medium stringency conditions and more preferably under high stringency conditions. Reference herein to low stringency conditions include and encompass from at least about 1% v/v to at least about 15% v/v formamide and from at least about 1 M to at least about 2 M salt for hybridization at 42° C., and at least about 1 M to at least about 2 M salt for washing at 42° C. Low stringency conditions also may include 1% Bovine Serum Albumin (BSA), 1 mM EDTA, 0.5 M NaHPO4 (pH 7.2), 7% SDS for hybridization at 65° C., and (i) 2×SSC, 0.1% SDS; or (ii) 0.5% BSA, 1 mM EDTA, 40 mM NaHPO4 (pH 7.2), 5% SDS for washing at room temperature. Medium stringency conditions include and encompass from at least about 16% v/v to at least about 30% v/v formamide and from at least about 0.5 M to at least about 0.9 M salt for hybridization at 42° C., and at least about 0.5 M to at least about 0.9 M salt for washing at 42° C. Medium stringency conditions also may include 1% Bovine Serum Albumin (BSA), 1 mM EDTA, 0.5 M NaHPO4 (pH 7.2), 7% SDS for hybridization at 65° C., and (i) 2×SSC, 0.1% SDS; or (ii) 0.5% BSA, 1 mM EDTA, 40 mM NaHPO4 (pH 7.2), 5% SDS for washing at 42° C. High stringency conditions include and encompass from at least about 31% v/v to at least about 50% v/v formamide and from at least about 0.01 M to at least about 0.15 M salt for hybridization at 42° C., and at least about 0.01 M to at least about 0.15 M salt for washing at 42° C. High stringency conditions also may include 1% BSA, 1 mM EDTA, 0.5 M NaHPO4 (pH 7.2), 7% SDS for hybridization at 65° C., and (i) 0.2×SSC, 0.1% SDS; or (ii) 0.5% BSA, 1 mM EDTA, 40 mM NaHPO4 (pH 7.2), 1% SDS for washing at a temperature in excess of 65° C. One embodiment of high stringency conditions includes hybridizing in 6×SSC at about 45° C., followed by one or more washes in 0.2×SSC, 0.1% SDS at 65° C. One embodiment of very high stringency conditions includes hybridizing 0.5 M sodium phosphate, 7% SDS at 65° C., followed by one or more washes at 0.2×SSC, 1% SDS at 65° C. Other stringent conditions are well known in the art. A skilled addressee will recognize that various factors can be manipulated to optimize the specificity of the hybridization. Optimization of the stringency of the final washes can serve to ensure a high degree of hybridization. For detailed examples, see CURRENT PROTOCOLS IN MOLECULAR BIOLOGY (supra) at pages 2.10.1 to 2.10.16 and MOLECULAR CLONING. A LABORATORY MANUAL (Sambrook, et al., eds.) (Cold Spring Harbor Press 1989) at sections 1.101 to 1.104.

The term “targeting” and its grammatical equivalents refer to the preferential movement in vivo of an agent (e.g., a biologically active agent) to a target site (e.g., a cell or tissue), or preferential accumulation in vivo of an agent at a target site (e.g., a cell or tissue), as compared to a control site. A target site may comprise a cell expressing a target, i.e., an intended site for accumulation of a targeting moiety or a conjugate comprising the targeting moiety and an agent, suitably a biologically active agent. A control site may comprise a cell that substantially lacks expression of the target and which therefore substantially lacks binding and/or accumulation of an administered targeting moiety or a conjugate comprising the targeting moiety and an agent, suitably a biologically active agent. Alternatively, a target site may comprise a cell (e.g., an immune cell such as an APC) that has selective affinity for, or otherwise selectively binds to, an agent disclosed herein (e.g., an agent associated with a particle), and which suitably enters the interior of the cell. Selective binding generally refers to a preferential localization of a targeting moiety or a conjugate comprising the targeting moiety and an agent, suitably a biologically active agent, such that an amount of targeting moiety or conjugate at a target site is at least about 2-fold, 3-fold, 4-fold, 5-fold, 6-fold, 7-fold, 8-fold, 9-fold, 10-fold, 20-fold, 30-fold, 40-fold, 50-fold, or 100-fold greater than an amount of targeting moiety or conjugate at a control site. In representative embodiments of this type, the target site is an immune cell and the control site is other than an immune cell. In other representative embodiments, the target site is an immune cell of a first type and the control site is an immune cell of a second type. In illustrative examples of this type, the immune cell of the first type is an APC (e.g., a macrophage) and the immune cell of the second type is a T cell. In other illustrative examples, the immune cell of the first type is a T cell and the immune cell of the second type is an APC (e.g., a macrophage).

The term “targeting moiety” refers to a structure that has a selective affinity for a target molecule relative to other non-target molecules. The targeting moiety binds to a target molecule. A targeting moiety may include, for example, an antibody, a peptide, a ligand, a receptor, or a binding portion thereof. The target molecule may be an antigen, such as a biological receptor or other structure suitably, for example, on the surface of a cell, within the cell membrane, within the cell, on the surface of a cellular organelle and the like; especially on the surface of a cell. The targeting moiety may be associated with any component of the therapeutic agent of the disclosure, such as, for example, the particle, or RL5P, RL5P-analog, RL5P analog-producing agent or RL5P-producing agent.

The term “therapeutic agent” as used herein refers to an agent that, when contacted with a cell (e.g., an immune cell such as an antigen-presenting cell), directly or indirectly leads to, creates and/or triggers one or more biological processes that inhibits or reduces the pro-inflammatory activity of the cell, and/or treats, prevents, inhibits or reduces, or slows the progression of, inflammation, as for example using the methods of the disclosure. The therapeutic agent may, in some embodiments, be in the form of a particle (or delivery vehicle) comprising or otherwise associated with an anti-inflammatory agent as described herein. In other embodiments, the therapeutic agent comprises, consists or consists essentially of an anti-inflammatory agent as described herein. The therapeutic agent may optionally comprise a targeting moiety as described herein.

As used herein, the term “therapeutic combination” refers to a combination of one or more active drug substances, i.e., compounds having a therapeutic utility when administered concurrently (i.e., combination therapy). Thus, the compounds may be in the form of a single composition, suitably comprising a mixture of the compounds, or in the form of separate compositions. Typically, each such compound in the therapeutic combinations of the present disclosure will be present in a pharmaceutical composition comprising that compound and a pharmaceutically acceptable carrier. The compounds in a therapeutic combination of the present disclosure are provided in dosage forms such that the beneficial effect of each therapeutic compound is realized by the subject at the desired time.

As used herein, the terms “treat”, “treatment”, “treating”, or “amelioration” refer to therapeutic treatments, wherein the object is to reverse, alleviate, ameliorate, inhibit, slow down or stop the progression or severity of a condition (e.g., inflammation) associated with a disease or disorder, e.g., an acute inflammatory condition such as SIRS, acute tissue injury, massive transfusion, etc. and chronic inflammatory conditions such as chronic liver disease, autoimmune disease, COPD, a neurodegenerative disease, a cardiovascular disease, etc. The term “treating” includes reducing or alleviating at least one adverse effect or symptom of a condition, disease or disorder. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of condition, disease or disorder, delay or slowing of progression of the condition, disease or disorder, amelioration or palliation of the state of the condition, disease or disorder, remission (whether partial or total), and/or decreased mortality, whether detectable or undetectable. The term “treatment” of a condition, disease or disorder also includes providing relief from the symptoms or side-effects of the condition, disease or disorder (including palliative treatment).

A “5′ untranslated region” (UTR) refers to a region of an mRNA that is directly upstream (i.e., 5′) from the start codon (i.e., the first codon of an mRNA transcript translated by a ribosome) that does not encode a polypeptide.

A “3′ untranslated region” (UTR) refers to a region of an mRNA that is directly downstream (i.e., 3′) from the stop codon (i.e., the codon of an mRNA transcript that signals a termination of translation) that does not encode a polypeptide.

As used herein, underscoring or italicizing the name of a gene shall indicate the gene, in contrast to its protein product, which is indicated by the name of the gene in the absence of any underscoring or italicizing. For example, “6PGD” shall mean the gene encoding the enzyme 6-phosphogluconate dehydrogenase, whereas “6PGD” shall indicate the product generated from transcription and translation and/or alternative splicing of the “6PGD” gene (i.e., the enzyme 6-phosphogluconate dehydrogenase).

It should be noted that the specific embodiments disclosed herein are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “some embodiments”, “certain embodiments”, “specific embodiments”, “one embodiment”, “an embodiment”, etc. means that a particular feature, structure or characteristic described in connection with the embodiment(s) is included in at least one embodiment of the present disclosure. Thus, appearances of the phrases “some embodiments”, “certain embodiments”, “specific embodiments”, “one embodiment”, “an embodiment”, etc. in various places throughout this specification are not necessarily all referring to the same embodiment(s), but may refer to the same embodiment(s). Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the disclosure. For example, in the appended claims, any of the claimed embodiments can be used in any combination. Thus, each embodiment described herein is to be applied mutatis mutandis to each and every embodiment unless specifically stated otherwise.

### 2. Therapeutic Agents for Reducing Inflammation

Aspects and embodiments disclosed herein relate to methods, agents and compositions for inhibiting or reducing pro-inflammatory activity of an antigen-presenting cell (APC), or for treating, inhibiting or reducing, or slowing the progression of, inflammation in a subject. These methods, agents and compositions involve the introduction of D-ribulose-5-phosphate (RL5P), a RL5P analog, a RL5P analog-producing agent or a RL5P-producing agent (e.g., 6PGD and a nucleic acid molecule from which 6PGD is producible), as anti-inflammatory agents, into an APC. Delivery is suitably carried out, in some embodiments, using a particle as a delivery vehicle that is capable of being taken up by an APC, and that comprises or is otherwise associated with one or more anti-inflammatory agents as a cargo of the delivery vehicle. The APC is suitably a monocyte such as macrophage, a dendritic cell or a B-cell, preferably a macrophage.

The present disclosure further relates to methods, agents and compositions for inhibiting or reducing pro-inflammatory activity of immune cells generally, including antigen-presenting cells, or for treating, inhibiting or reducing, or slowing the progression of, inflammation in a subject. These methods, agents and compositions involve the co-introduction of D-ribulose-5-phosphate (RL5P), a RL5P analog, a RL5P analog-producing agent or a RL5P-producing agent (e.g., 6PGD and a nucleic acid molecule from which 6PGD is producible) and an HDAC7 polypeptide-producing agent (e.g., an HDAC7 polypeptide or a nucleic acid molecule from which an HDAC7 polypeptide is producible), as anti-inflammatory agents, into the immune cell.

In some embodiments, the therapeutic agent targets an immune cell. In exemplary embodiments, the therapeutic agent is or comprises an APC-targeting agent.

### 2.1 Anti-Inflammatory Agents

### 2.1.1 RL5P and RL5P Analogs

In some embodiments, RL5P or an RL5P analog is delivered directly to an immune cell such as an APC or is used as a cargo of a particle (e.g., delivery vehicle) or targeting moiety. RL5P is available from several commercial suppliers including Simulationtech Inc. (Texas, USA), Biosynth (Berkshire, UK), CymitQuimica (Barcelona, Spain) and eNovation Chemicals LLC (New Jersey, USA) or may be prepared by any suitable technique, including for example according to the method of Pontremoli et al. (J Biol Chem. 1962; 237(3) 643-645) and the synthetic protocol presented in Example 8 herein.

Representative RL5P analogs include both functional and structural analogs that, when contacted with an immune cell such as an APC, ultimately lead to, create and/or trigger one or more biological processes that inhibit or reduce the pro-inflammatory activity of an immune cell (e.g., an APC), and/or treat, prevent, inhibit or reduce, or slow the progression of, inflammation, wherein the one or more biological processes correspond to a biological process that is ordinarily led to, created by and/or triggered by RL5P.

### 2.1.2 RL5P-Producing Agent, or a RL5P Analog-Producing Agent

The RL5P-producing agent is a molecule, preferably an enzyme, that is capable of converting 6-phosphate-D-gluconate (or an analog thereof) into RL5P or an RL5P analog. In a native environment, this conversion occurs in the pentose phosphate pathway and the enzyme responsible is 6PGD. However, in an immune cell such as an APC where there is a source of 6-phosphate-D-gluconate, any active agent or molecule that can covert the 6-phosphate-D-gluconate to RL5P or a functional RL5P analog may be sufficient to lead to, create and/or trigger the one or more biological processes that inhibit or reduce the pro-inflammatory activity of an immune cell (e.g., an APC), and/or treat, prevent, inhibit or reduce, or slow the progression of, inflammation, in accordance, for example, with the methods and compositions of the present disclosure. An exemplary RL5P-producing agent or an RL5P analog-producing agent may be a recombinant enzyme derived from, or modeled from, the native enzyme, 6PGD, or an active site, domain or region of 6PGD. In another example, RL5P-producing agent or an RL5P analog-producing agent may be a functional analog of 6PGD, or may be an protein with an active site that is functionally analogous to one of more active site, domains or regions from 6PGD.

### 2.1.3 6PGD and 6PGD-Encoding Nucleic Acid Molecules

In some embodiments, the RL5P-producing agent is 6PGD or a nucleic acid molecule from which 6PGD is producible.

Representative 6PGD polypeptides comprise, consist or consist essentially of an amino acid sequence selected from:
MAQADIALIGLAVMGQNLILNMNDHGFVVCAFNRTVSKVDDFLANEAKGTKVVGAQSLK EMVSKLKKPRRIILLVKAGQAVDDFIEKLVPLLDTGDIIIDGGNSEYRDTTRRCRDLKAKGILFVGSGVSGGEEGA RYGPSLMPGGNKEAWPHIKTIFQGIAAKVGTGEPCCDWVGDEGAGHFVKMVHNGIEYGDMQLICEAYHLMKDV LGMAQDEMAQAFEDWNKTELDSFLIEITANILKFQDTDGKHLLPKIRDSAGQKGTGKWTAISALEYGVPVTLIGE AVFARCLSSLKDERIQASKKLKGPQKFQFDGDKKSFLEDIRKALYASKIISYAQGFMLLRQAATEFGWTLNYGGI ALMWRGGCIIRSVFLGKIKDAFDRNPELQNLLLDDFFKSAVENCQDSWRRAVSTGVQAGIPMPCFTTALSFYDG YRHEMLPASLIQAQRDYFGAHTYELLAKPGQFIHTNWTGHGGTVSSSSYNA [SEQ ID NO: 1], corresponding to the full-length human 6PGD amino acid sequence set forth in UniProt Accession No. P52209;
MGQNLILNMNDHGFVVCAFNRTVSKVDDFLANEAKGTKVVGAQSLKEMVSKLKKPRRIIL LVKAGQAVDDFIEKLVPLLDTGDIIIDGGNSEYRDTTRRCRDLKAKGILFVGSGVSGGEEGARYGPSLMPGGNKE AWPHIKTIFQGIAAKVGTGEPCCDWVGDEGAGHFVKMVHNGIEYGDMQLICEAYHLMKDVLGMAQDEMAQAF EDWNKTELDSFLIEITANILKFQDTDGKHLLPKIRDSAGQKGTGKWTAISALEYGVPVTLIGEAVFARCLSSLKDE RIQASKKLKGPQKFQFDGDKKSFLEDIRKALYASKIISYAQGFMLLRQAATEFGWTLNYGGIALMWRGGCIIRSV FLGKIKDAFDRNPELQNLLLDDFFKSAVENCQDSWRRAVSTGVQAGIPMPCFTTALSFYDGYRHEMLPASLIQAQ RDYFGAHTYELLAKPGQFIHTNWTGHG [SEQ ID NO:2], corresponding to the NADP-dependent phosphogluconate dehydrogenase domain of SEQ ID NO:1;
MAQADIALIGLAVMGQNLILNMNDHGFVVCAFNRTVSKVDDFLANEAKGTKVVGAQSLK DMVSKLKKPRRVILLVKAGQAVDDFIEKLVPLLDTGDIIIDGGNSEYRDTTRRCRDLKAKGILFVGSGVSGGEEG ARYGPSLMPGGNKEAWPHIKAIFQAIAAKVGTGEPCCDWVGDEGAGHFVKMVHNGIEYGDMQLICEAYHLMKD VLGMRHEEMAQAFEEWNKTELDSFLIEITANILKYRDTDGKELLPKIRDSAGQKGTGKWTAISALEYGMPVTLIG EAVFARCLSSLKEERVQASQKLKGPKVVQLEGSKKSFLEDIRKALYASKIISYAQGFMLLRQAATEFGWTLNYGG IALMWRGGCIIRSVFLGKIKDAFERNPELQNLLLDDFFKSAVDNCQDSWRRVISTGVQAGIPMPCFTTALSFYDG YRHEMLPANLIQAQRDYFGAHTYELLTKPGEFIHTNWTGHGGSVSSSSYNA [SEQ ID NO:3], corresponding to the full-length mouse 6PGD amino acid sequence set forth in UniProt Accession No. Q9DCD0;
MGQNLILNMNDHGFVVCAFNRTVSKVDDFLANEAKGTKVVGAQSLKDMVSKLKKPRRVI LLVKAGQAVDDFIEKLVPLLDTGDIIIDGGNSEYRDTTRRCRDLKAKGILFVGSGVSGGEEGARYGPSLMPGGNK EAWPHIKAIFQAIAAKVGTGEPCCDWVGDEGAGHFVKMVHNGIEYGDMQLICEAYHLMKDVLGMRHEEMAQA FEEWNKTELDSFLIEITANILKYRDTDGKELLPKIRDSAGQKGTGKWTAISALEYGMPVTLIGEAVFARCLSSLKE ERVQASQKLKGPKVVQLEGSKKSFLEDIRKALYASKIISYAQGFMLLRQAATEFGWTLNYGGIALMWRGGCIIRS VFLGKIKDAFERNPELQNLLLDDFFKSAVDNCQDSWRRVISTGVQAGIPMPCFTTALSFYDGYRHEMLPANLIQA QRDYFGAHTYELLTKPGEFIHTNWT [SEQ ID NO:4], corresponding to the NADP-dependent phosphogluconate dehydrogenase domain of SEQ ID NO:3; and
an amino acid sequence corresponding to the amino acid sequence set forth in any one of SEQ ID NO:1-4, representative examples of which display at least about 70, 71, 72, 73, 74, 75, 76, 77, 78, 79, 80, 81, 82, 83, 84, 85, 86, 97, 88, 89, 90, 91, 92, 93, 94, 95, 96, 97, 98 or 99% sequence similarity or identity to a reference amino acid sequence selected from any one of SEQ ID NO:1-4.

Illustrative 6PGD-encoding nucleic acid molecules may comprise, consist or consist essentially of a nucleic acid sequence selected from:
atggcccaagctgacatcgcgctgatcggattggccgtcatgggccagaacttaattctgaacatgaatgaccac ggctttgtggtctgtgcttttaataggactgtctccaaagttgatgatttcttggccaatgaggcaaagggaaccaaagtggtgggtgcccagt ccctgaaagagatggtctccaagctgaagaagccccggcggatcatcctcctggtgaaggctgggcaagctgtggatgatttcatcgagaa attggtaccattgttggatactggtgacatcatcattgacggaggaaattctgaatatagggacaccacaagacggtgccgagacctcaagg ccaagggaattttatttgtggggagcggagtcagtggtggagaggaaggggcccggtatggcccatcgctcatgccaggagggaacaaag aagcgtggccccacatcaagaccatcttccaaggcattgctgcaaaagtgggaactggagaaccctgctgtgactgggtgggagatgaggg agcaggccacttcgtgaagatggtgcacaacgggatagagtatggggacatgcagctgatctgtgaggcataccacctgatgaaagacgtg ctgggcatggcgcaggacgagatggcccaggcctttgaggattggaataagacagagctagactcattcctgattgaaatcacagccaatat tctcaagttccaagacaccgatggcaaacacctgctgccaaagatcagggacagcgcggggcagaagggcacagggaagtggaccgcca tctccgccctggaatacggcgtacccgtcaccctcattggagaagctgtctttgctcggtgcttatcatctctgaaggatgagagaattcaagct agcaaaaagctgaagggtccccagaagttccagtttgatggtgataagaaatcattcctggaggacattcggaaggcactctacgcttccaa gatcatctcttacgctcaaggctttatgctgctaaggcaggcagccaccgagtttggctggactctcaattatggtggcatcgccctgatgtgga gagggggctgcatcattagaagtgtattcctaggaaagataaaggatgcatttgatcgaaacccggaacttcagaacctcctactggacgac ttctttaagtcagctgttgaaaactgccaggactcctggcggcgggcagtcagcactggggtccaggctggcattcccatgccctgttttacca ctgccctctccttctatgacgggtacagacatgagatgcttccagccagcctcatccaggctcagcgggattacttcggggctcacacctatga actcttggccaaaccagggcagtttatccacaccaactggacaggccatggtggcaccgtgtcatcctcgtcatacaatgcc [SEQ ID NO:5], corresponding to the full-length human PGD coding sequence set forth in GenBank Accession No. NM_002631;
atgggccagaacttaattctgaacatgaatgaccacggctttgtggtctgtgcttttaataggactgtctccaaagtt gatgatttcttggccaatgaggcaaagggaaccaaagtggtgggtgcccagtccctgaaagagatggtctccaagctgaagaagccccggc ggatcatcctcctggtgaaggctgggcaagctgtggatgatttcatcgagaaattggtaccattgttggatactggtgacatcatcattgacgg aggaaattctgaatatagggacaccacaagacggtgccgagacctcaaggccaagggaattttatttgtggggagcggagtcagtggtgg agaggaaggggcccggtatggcccatcgctcatgccaggagggaacaaagaagcgtggccccacatcaagaccatcttccaaggcattgc tgcaaaagtgggaactggagaaccctgctgtgactgggtgggagatgagggagcaggccacttcgtgaagatggtgcacaacgggatag agtatggggacatgcagctgatctgtgaggcataccacctgatgaaagacgtgctgggcatggcgcaggacgagatggcccaggcctttga ggattggaataagacagagctagactcattcctgattgaaatcacagccaatattctcaagttccaagacaccgatggcaaacacctgctgcc aaagatcagggacagcgcggggcagaagggcacagggaagtggaccgccatctccgccctggaatacggcgtacccgtcaccctcattgg agaagctgtctttgctcggtgcttatcatctctgaaggatgagagaattcaagctagcaaaaagctgaagggtccccagaagttccagtttga tggtgataagaaatcattcctggaggacattcggaaggcactctacgcttccaagatcatctcttacgctcaaggctttatgctgctaaggcag gcagccaccgagtttggctggactctcaattatggtggcatcgccctgatgtggagagggggctgcatcattagaagtgtattcctaggaaag ataaaggatgcatttgatcgaaacccggaacttcagaacctcctactggacgacttctttaagtcagctgttgaaaactgccaggactcctggc ggcgggcagtcagcactggggtccaggctggcattcccatgccctgttttaccactgccctctccttctatgacgggtacagacatgagatgctt ccagccagcctcatccaggctcagcgggattacttcggggctcacacctatgaactcttggccaaaccagggcagtttatccacaccaactgg acaggccatggt [SEQ ID NO:6], corresponding to the NADP-dependent phosphogluconate dehydrogenase domain coding portion of SEQ ID NO:5;
atggcccaagctgacattgcactgatcggactggctgtcatgggccagaacttaattttgaacatgaatgatcatg gatttgtggtctgtgctttcaataggacagtctccaaagtcgatgacttcttggccaacgaggcgaagggcaccaaggtggttggtgcacagt ccttaaaggacatggtctccaaactaaagaagccccgccgggtcatcctgcttgtgaaggccgggcaagccgtggatgatttcatcgagaaa ttagtacccttgttggacacgggtgacatcatcatcgatggaggaaattctgaataccgggacacgacaagaagatgccgggacctcaagg ccaagggcatcttgtttgtggggagcggagtcagtggtggtgaggaaggggctcggtacgggccgtcactcatgccaggagggaacaaag aggcttggccccacatcaaggcgatcttccaagccatcgctgcaaaagtgggaaccggagaaccctgctgtgactgggtgggagatgagg gggcggggcactttgtgaagatggtgcacaacgggatagagtatggagacatgcagctcatctgtgaggcttaccatttgatgaaggatgtt ctgggcatgcggcatgaggagatggctcaggcatttgaagaatggaacaagacagagctggactcattcctgattgaaatcactgctaacat tctcaagtaccgggacactgacggcaaagagctgttgccaaagatccgggacagtgctgggcagaagggcactgggaagtggaccgccat ctcggcgctggagtacggcatgcccgtcaccctcattggagaagctgtctttgctcggtgcttgtcctctctgaaggaggagcgggttcaggcc agccaaaagctgaagggtcctaaggtggtccagctggaaggcagtaagaagtcattcctggaggacatccgtaaggccctctatgcttccaa gatcatctcctacgcccaaggctttatgctgctcagacaggcagccactgagtttggctggaccctcaattatggcggcattgccctgatgtgg agagggggctgcatcatccgaagtgtgttcctgggaaaaattaaagatgcatttgagcgaaacccagaacttcagaacctactgctagatga cttctttaagtcagcagttgacaactgccaggactcctggcggcgggtgatcagcactggggtgcaagcaggcattcccatgccctgcttcact actgccctctccttctatgatgggtacagacacgagatgctgccagcaaacctcatccaggctcaacgggattactttggggctcacacctatg aactcttaaccaaaccgggagaatttatccacaccaactggacgggccacgggggcagtgtgtcatcctcttcatacaatgcc [SEQ ID NO:7], corresponding to the full-length mouse Pgd coding sequence set forth in GenBank Accession No. NM_001081274;
atgggccagaacttaattttgaacatgaatgatcatggatttgtggtctgtgctttcaataggacagtctccaaagt cgatgacttcttggccaacgaggcgaagggcaccaaggtggttggtgcacagtccttaaaggacatggtctccaaactaaagaagccccgc cgggtcatcctgcttgtgaaggccgggcaagccgtggatgatttcatcgagaaattagtacccttgttggacacgggtgacatcatcatcgat ggaggaaattctgaataccgggacacgacaagaagatgccgggacctcaaggccaagggcatcttgtttgtggggagcggagtcagtggt ggtgaggaaggggctcggtacgggccgtcactcatgccaggagggaacaaagaggcttggccccacatcaaggcgatcttccaagccatc gctgcaaaagtgggaaccggagaaccctgctgtgactgggtgggagatgagggggcggggcactttgtgaagatggtgcacaacgggat agagtatggagacatgcagctcatctgtgaggcttaccatttgatgaaggatgttctgggcatgcggcatgaggagatggctcaggcatttg aagaatggaacaagacagagctggactcattcctgattgaaatcactgctaacattctcaagtaccgggacactgacggcaaagagctgttg ccaaagatccgggacagtgctgggcagaagggcactgggaagtggaccgccatctcggcgctggagtacggcatgcccgtcaccctcattg gagaagctgtctttgctcggtgcttgtcctctctgaaggaggagcgggttcaggccagccaaaagctgaagggtcctaaggtggtccagctg gaaggcagtaagaagtcattcctggaggacatccgtaaggccctctatgcttccaagatcatctcctacgcccaaggctttatgctgctcagac aggcagccactgagtttggctggaccctcaattatggcggcattgccctgatgtggagagggggctgcatcatccgaagtgtgttcctgggaa aaattaaagatgcatttgagcgaaacccagaacttcagaacctactgctagatgacttctttaagtcagcagttgacaactgccaggactcctg gcggcgggtgatcagcactggggtgcaagcaggcattcccatgccctgcttcactactgccctctccttctatgatgggtacagacacgagat gctgccagcaaacctcatccaggctcaacgggattactttggggctcacacctatgaactcttaaccaaaccgggagaatttatccacaccaa ctggacg [SEQ ID NO:8], corresponding to the NADP-dependent phosphogluconate dehydrogenase domain coding portion of SEQ ID NO:7; and
a nucleic acid sequence corresponding to any one of the nucleic acid sequences set forth in SEQ ID NO:5-8, representative examples of which display at least about 70, 71, 72, 73, 74, 75, 76, 77, 78, 79, 80, 81, 82, 83, 84, 85, 86, 97, 88, 89, 90, 91, 92, 93, 94, 95, 96, 97, 98 or 99% sequence identity to a reference nucleic acid sequence selected from any one of SEQ ID NO:5-8, or hybridize to a reference nucleic acid sequence selected from any one of SEQ ID NO:5-8 under at least medium stringency, at least high stringency or very high stringency conditions.

The 6PGD-encoding nucleic acid molecules may be in the form a ribonucleic acid (RNA), or deoxyribonucleic acid (DNA), for endogenous production of 6PGD in a chosen cell (e.g., an immune cell such as an APC).

In some embodiments, the nucleic acid molecules comprise an RNA, suitably a messenger RNA (mRNA) polynucleotide, comprising an open reading frame (ORF) encoding a 6PGD polypeptide (i.e., a coding sequence for a 6PGD polypeptide). In other embodiments, the nucleic acid molecules are in the form of a nucleic acid construct comprising a DNA polynucleotide that comprises an ORF encoding a 6PGD polypeptide, in operable connection with a regulatory element that is suitably operable in a chosen cell (e.g., an immune cell such as an APC).

### 2.1.4 6PGD Activity-Enhancing Agents

In accordance with the present disclosure, the activity of 6PGD may be enhanced by an HDAC7 polypeptide or a nucleic acid molecule from which an HDAC7 polypeptide is producible.

Representative HDAC7 polypeptides comprise, consist or consist essentially of an amino acid sequence selected from:
MHSPGADGTQVSPGAHYCSPTGAGCPRPCADTPGPQPQPMDLRVGQRPPVEPPPEPTLLA LQRPQRLHHHLFLAGLQQQRSVEPMRLSMDTPMPELQVGPQEQELRQLLHKDKSKRSAVASSVVKQKLAEVILK KQQAALERTVHPNSPGIPYRTLEPLETEGATRSMLSSFLPPVPSLPSDPPEHFPLRKTVSEPNLKLRYKPKKSLERR KNPLLRKESAPPSLRRRPAETLGDSSPSSSSTPASGCSSPNDSEHGPNPILGSEALLGQRLRLQETSVAPFALPTV SLLPAITLGLPAPARADSDRRTHPTLGPRGPILGSPHTPLFLPHGLEPEAGGTLPSRLQPILLLDPSGSHAPLLTVPG LGPLPFHFAQSLMTTERLSGSGLHWPLSRTRSEPLPPSATAPPPPGPMQPRLEQLKTHVQVIKRSAKPSEKPRLRQ IPSAEDLETDGGGPGQVVDDGLEHRELGHGQPEARGPAPLQQHPQVLLWEQQRLAGRLPRGSTGDTVLLPLAQ GGHRPLSRAQSSPAAPASLSAPEPASQARVLSSSETPARTLPFTTGLIYDSVMLKHQCSCGDNSRHPEHAGRIQS IWSRLQERGLRSQCECLRGRKASLEELQSVHSERHVLLYGTNPLSRLKLDNGKLAGLLAQRMFVMLPCGGVGVD TDTIWNELHSSNAARWAAGSVTDLAFKVASRELKNGFAVVRPPGHHADHSTAMGFCFFNSVAIACRQLQQQSK ASKILIVDWDVHHGNGTQQTFYQDPSVLYISLHRHDDGNFFPGSGAVDEVGAGSGEGFNVNVAWAGGLDPPM GDPEYLAAFRIVVMPIAREFSPDLVLVSAGFDAAEGHPAPLGGYHVSAKCFGYMTQQLMNLAGGAVVLALEGGH DLTAICDASEACVAALLGNRVDPLSEEGWKQKPNLNAIRSLEAVIRVHSKYWGCMQRLASCPDSWVPRVPGAD KEEVEAVTALASLSVGILAEDRPSEQLVEEEEPMNL [SEQ ID NO:9], corresponding to the full-length human HDAC7 isoform A amino acid sequence set forth in GenPept Accession No. NP_056216, version NP_056216.2;
MDLRVGQRPPVEPPPEPTLLALQRPQRLHHHLFLAGLQQQRSVEPMRLSMDTPMPELQVG PQEQELRQLLHKDKSKRSAVASSVVKQKLAEVILKKQQAALERTVHPNSPGIPYRTLEPLETEGATRSMLSSFLPP VPSLPSDPPEHFPLRKTVSEPNLKLRYKPKKSLERRKNPLLRKESAPPSLRRRPAETLGDSSPSSSSTPASGCSSP NDSEHGPNPILGSEALLGQRLRLQETSVAPFALPTVSLLPAITLGLPAPARADSDRRTHPTLGPRGPILGSPHTPLF LPHGLEPEAGGTLPSRLQPILLLDPSGSHAPLLTVPGLGPLPFHFAQSLMTTERLSGSGLHWPLSRTRSEPLPPSAT APPPPGPMQPRLEQLKTHVQVIKRSAKPSEKPRLRQIPSAEDLETDGGGPGQVVDDGLEHRELGHGQPEARGPA PLQQHPQVLLWEQQRLAGRLPRGSTGDTVLLPLAQGGHRPLSRAQSSPAAPASLSAPEPASQARVLSSSETPAR TLPFTTGLIYDSVMLKHQCSCGDNSRHPEHAGRIQSIWSRLQERGLRSQCECLRGRKASLEELQSVHSERHVLL YGTNPLSRLKLDNGKLAGLLAQRMFVMLPCGGVGVDTDTIWNELHSSNAARWAAGSVTDLAFKVASRELKNGF AVVRPPGHHADHSTAMGFCFFNSVAIACRQLQQQSKASKILIVDWDVHHGNGTQQTFYQDPSVLYISLHRHDD GNFFPGSGAVDEVGAGSGEGFNVNVAWAGGLDPPMGDPEYLAAFRIVVMPIAREFSPDLVLVSAGFDAAEGHP APLGGYHVSAKCFGYMTQQLMNLAGGAVVLALEGGHDLTAICDASEACVAALLGNRVDPLSEEGWKQKPNLNA IRSLEAVIRVHSKYWGCMQRLASCPDSWVPRVPGADKEEVEAVTALASLSVGILAEDRPSEQLVEEEEPMNL [SEQ ID NO:10], corresponding to the human HDAC7 isoform A amino acid sequence of SEQ ID NO:9 lacking the first 39 amino acids of that sequence;
MDLRVGQRPPVEPPPEPTLLALQRPQRLHHHLFLAGLQQQRSVEPMRLSMDTPMPELQVG PQEQELRQLLHKDKSKRSAVASSVVKQKLAEVILKKQQAALERTVHPNSPGIPYRTLEPLETEGATRSMLSSFLPP VPSLPSDPPEHFPLRKTVSEPNLKLRYKPKKSLERRKNPLLRKESAPPSLRRRPAETLGDSSPSSSSTPASGCSSP NDSEHGPNPILGSEALLGQRLRLQETSVAPFALPTVSLLPAITLGLPAPARADSDRRTHPTLGPRGPILGSPHTPLF LPHGLEPEAGGTLPSRLQPILLLDPSGSHAPLLTVPGLGPLPFHFAQSLMTTERLSGSGLHWPLSRTRSEPLPPSAT APPPPGPMQPRLEQLKTHVQVIKRSAKPSEKPRLRQIPSAEDLETDGGGPGQVVDDGLEHRELGHGQPEARGPA PLQQHPQVLLWEQQRLAGRLPRGSTGDTVLLPLAQGGHRPLSRAQSSPAAPASLSAPEPASQARVLSSSET [SEQ ID NO:11], which: (1) lacks the first 39 amino acids of the full-length human HDAC7 isoform A amino acid sequence of SEQ ID NO:9; (2) comprises the PHA03247 super family motif (from about amino acid 81 through about amino acid 522 of the full-length human HDAC7 amino acid sequence set forth in SEQ ID NO:9) corresponding to the herpes simplex virus-1 UL36 large tegument protein, which is known to comprise deubiquitinating activity, and (3) lacks a C-terminal portion of human HDAC7 (spanning from about amino acid 551 through about amino acid 991), which C-terminal portion comprises the HDAC7 deacetylase enzyme domain;
MHSPGAGCPALQPDTPGSQPQPMDLRVGQRPTVEPPPEPALLTLQHPQRLHRHLFLAGLH QQQRSAEPMRLSMDPPMPELQGGQQEQELRQLLNKDKSKRSAVASSVVKQKLAEVILKKQQAALERTVHPSSP SIPYRTLEPLDTEGAARSVLSSFLPPVPSLPTEPPEHFPLRKTVSEPNLKLRYKPKKSLERRKNPLLRKESAPPSLRR RPAETLGDSSPSSSSTPASGCSSPNDSEHGPNPALGSEADGDRRTHSTLGPRGPVLGNPHAPLFLHHGLEPEAG GTLPSRLQPILLLDPSVSHAPLWTVPGLGPLPFHFAQPLLTTERLSGSGLHRPLNRTRSEPLPPSATASPLLAPLQP RQDRLKPHVQLIKPAISPPQRPAKPSEKPRLRQIPSAEDLETDGGGVGPMANDGLEHRESGRGPPEGRGSISLQ QHQQVPPWEQQHLAGRLSQGSPGDSVLIPLAQVGHRPLSRTQSSPAAPVSMLSPEPTCQTQVLNSSETPATGLV YDSVMLKHQCSCGDNSKHPEHAGRIQSIWSRLQERGLRSQCECLRGRKASLEELQSVHSERHVLLYGTNPLSRL KLDNGKLTGLLAQRTFVMLPCGGVGVDTDTIWNELHSSNAARWAAGSVTDLAFKVASRELKNGFAVVRPPGHH ADHSTAMGFCFFNSVAIACRQLQQHGKASKILIVDWDVHHGNGTQQTFYQDPSVLYISLHRHDDGNFFPGSGA VDEVGTGSGEGFNVNVAWAGGLDPPMGDPEYLAAFRIVVMPIAREFAPDLVLVSAGFDAAEGHPAPLGGYHVSA KCFGYMTQQLMNLAGGAVVLALEGGHDLTAICDASEACVAALLGNKVDPLSEESWKQKPNLSAIRSLEAVVRVH RKYWGCMQRLASCPDSWLPRVPGADAEVEAVTALASLSVGILAEDRPSERLVEEEEPMNL [SEQ ID NO: 12], corresponding to a full-length mouse HDAC7 isoform 4 amino acid sequence set for in NCBI Reference Sequence: NP_062518.2;
MDLRVGQRPTVEPPPEPALLTLQHPQRLHRHLFLAGLHQQQRSAEPMRLSMDPPMPELQG GQQEQELRQLLNKDKSKRSAVASSVVKQKLAEVILKKQQAALERTVHPSSPSIPYRTLEPLDTEGAARSVLSSFL PPVPSLPTEPPEHFPLRKTVSEPNLKLRYKPKKSLERRKNPLLRKESAPPSLRRRPAETLGDSSPSSSSTPASGCSS PNDSEHGPNPALGSEADGDRRTHSTLGPRGPVLGNPHAPLFLHHGLEPEAGGTLPSRLQPILLLDPSVSHAPLWT VPGLGPLPFHFAQPLLTTERLSGSGLHRPLNRTRSEPLPPSATASPLLAPLQPRQDRLKPHVQLIKPAISPPQRPAK PSEKPRLRQIPSAEDLETDGGGVGPMANDGLEHRESGRGPPEGRGSISLQQHQQVPPWEQQHLAGRLSQGSP GDSVLIPLAQVGHRPLSRTQSSPAAPVSMLSPEPTCQTQVLNSSETPATGLVYDSVMLKHQCSCGDNSKHPEHA GRIQSIWSRLQERGLRSQCECLRGRKASLEELQSVHSERHVLLYGTNPLSRLKLDNGKLTGLLAQRTFVMLPCG GVGVDTDTIWNELHSSNAARWAAGSVTDLAFKVASRELKNGFAVVRPPGHHADHSTAMGFCFFNSVAIACRQL QQHGKASKILIVDWDVHHGNGTQQTFYQDPSVLYISLHRHDDGNFFPGSGAVDEVGTGSGEGFNVNVAWAGG LDPPMGDPEYLAAFRIVVMPIAREFAPDLVLVSAGFDAAEGHPAPLGGYHVSAKCFGYMTQQLMNLAGGAVVLAL EGGHDLTAICDASEACVAALLGNKVDPLSEESWKQKPNLSAIRSLEAVVRVHRKYWGCMQRLASCPDSWLPRV PGADAEVEAVTALASLSVGILAEDRPSERLVEEEEPMNL [SEQ ID NO: 13], corresponding to the mouse HDAC7 amino acid sequence of SEQ ID NO: 12 lacking the first 22 amino acids of that sequence; and
MDLRVGQRPTVEPPPEPALLTLQHPQRLHRHLFLAGLHQQQRSAEPMRLSMDPPMPELQG GQQEQELRQLLNKDKSKRSAVASSVVKQKLAEVILKKQQAALERTVHPSSPSIPYRTLEPLDTEGAARSVLSSFL PPVPSLPTEPPEHFPLRKTVSEPNLKLRYKPKKSLERRKNPLLRKESAPPSLRRRPAETLGDSSPSSSSTPASGCSS PNDSEHGPNPALGSEADGDRRTHSTLGPRGPVLGNPHAPLFLHHGLEPEAGGTLPSRLQPILLLDPSVSHAPLWT VPGLGPLPFHFAQPLLTTERLSGSGLHRPLNRTRSEPLPPSATASPLLAPLQPRQDRLKPHVQLIKPAISPPQRPAK PSEKPRLRQIPSAEDLETDGGGVGPMANDGLEHRESGRGPPEGRGSISLQQHQQVPPWEQQHLAGRLSQGSP GDSVLIPLAQVGHRPLSRTQSSPAAPVSMLSPEPTCQTQVLNSSETPATGLVYDSVMLKHQCSCGDNSKHPEHA GRIQSIWSRLQERGLRSQCECLRGRKASLEELQSVHSERHVLLYGTNPLSRLKLDNGKLTGLLAQRTFVMLPCG GVGVDTDTIWNELHSSNAARWAAGSVTDLAFKVASRELKNGFAVVRPPGHAADHSTAMGFCFFNSVAIACRQL QQHGKASKILIVDWDVHHGNGTQQTFYQDPSVLYISLHRHDDGNFFPGSGAVDEVGTGSGEGFNVNVAWAGG LDPPMGDPEYLAAFRIVVMPIAREFAPDLVLVSAGFDAAEGHPAPLGGYHVSAKCFGYMTQQLMNLAGGAVVLAL EGGHDLTAICDASEACVAALLGNKVDPLSEESWKQKPNLSAIRSLEAVVRVHRKYWGCMQRLASCPDSWLPRV PGADAEVEAVTALASLSVGILAEDRPSERLVEEEEPMNL [SEQ ID NO: 14], comprising an H-to-A substitution at residue 657, relative to the amino acid numbering of the full-length mouse HDAC7 amino acid sequence of SEQ ID NO:12, which abrogates HDAC7 deacetylase enzyme activity.
MDLRVGQRPTVEPPPEPALLTLQHPQRLHRHLFLAGLHQQQRSAEPMRLSMDPPMPELQG GQQEQELRQLLNKDKSKRSAVASSVVKQKLAEVILKKQQAALERTVHPSSPSIPYRTLEPLDTEGAARSVLSSFL PPVPSLPTEPPEHFPLRKTVSEPNLKLRYKPKKSLERRKNPLLRKESAPPSLRRRPAETLGDSSPSSSSTPASGCSS PNDSEHGPNPALGSEADGDRRTHSTLGPRGPVLGNPHAPLFLHHGLEPEAGGTLPSRLQPILLLDPSVSHAPLWT VPGLGPLPFHFAQPLLTTERLSGSGLHRPLNRTRSEPLPPSATASPLLAPLQPRQDRLKPHVQLIKPAISPPQRPAK PSEKPRLRQIPSAEDLETDGGGVGPMANDGLEHRESGRGPPEGRGSISLQQHQQVPPWEQQHLAGRLSQGSP GDSVLIPLAQVGHRPLSRTQSSPAAPVSMLSPEPTCQTQVLNSSET [SEQ ID NO:15], which: (1) lacks the first 22 amino acids of the full-length mouse HDAC7 amino acid sequence of SEQ ID NO:12; (2) comprises the PHA03247 super family motif (from amino acids 65 through 479 of the full-length mouse HDAC7 amino acid sequence set forth in SEQ ID NO:12) corresponding to the herpes simplex virus-1 UL36 large tegument protein, which is known to comprise deubiquitinating activity, and (3) lacks a C-terminal portion of mouse HDAC7 (spanning from about amino acid 505 through about amino acid 938), which C-terminal portion comprises the HDAC7 deacetylase enzyme domain;
an amino acid sequence corresponding to the amino acid sequence set forth in any one of SEQ ID NO:9-15, or to a domain or motif thereof (e.g., PHA03247 super family motif) representative examples of which display at least about 70, 71, 72, 73, 74, 75, 76, 77, 78, 79, 80, 81, 82, 83, 84, 85, 86, 97, 88, 89, 90, 91, 92, 93, 94, 95, 96, 97, 98 or 99% sequence similarity or identity to a reference amino acid sequence selected from any one of SEQ ID NO:9-15, or to a domain or motif thereof (e.g., PHA03247 super family motif).

Illustrative HDAC7 polypeptide-encoding nucleic acid molecules may comprise, consist or consist essentially of a nucleic acid sequence selected from:
atgcacagccccggcgctgatgggacccaggtgagcccgggtgcccactactgcagccccactggcgcaggctg ccccaggccctgtgcagacacaccaggccctcagccgcagcccatggacctgcgggtgggccagcggcccccagtggagcccccaccaga gcccacattgctggccctgcagcgtccccagcgcctgcaccaccacctcttcctagcaggcctgcagcagcagcgctcggtggagcccatga ggctctccatggacacgccgatgcccgagttgcaggtgggaccccaggaacaagagctgcggcagcttctccacaaggacaagagcaagc gaagtgctgtagccagcagcgtggtcaagcagaagctagcggaggtgattctgaaaaaacagcaggcggccctagaaagaacagtccat cccaacagccccggcattccctacagaaccctggagcccctggagacggaaggagccacccgctccatgctcagcagctttttgcctcctgttc ccagcctgcccagtgaccccccagagcacttccctctgcgcaagacagtctctgagcccaacctgaagctgcgctataagcccaagaagtccc tggagcggaggaagaatccactgctccgaaaggagagtgcgccccccagcctccggcggcggcccgcagagaccctcggagactcctccc caagtagtagcagcacgcccgcatcagggtgcagctcccccaatgacagcgagcacggccccaatcccatcctgggctcggaggcgctctt gggccagcggctgcggctgcaggagacttctgtggccccgttcgccttgccgacagtgtccttgctgcccgcaatcactctggggctgcccgc ccctgccagggctgacagtgaccgcaggacccatccgactctgggccctcgggggccaatcctggggagcccccacactcccctcttcctgcc ccatggcttggagcccgaggctgggggcaccttgccctctcgcctgcagcccattctcctcctggacccctcaggctctcatgccccgctgctga ctgtgcccgggcttgggcccttgcccttccactttgcccagtccttaatgaccaccgagcggctctctgggtcaggcctccactggccactgagc cggactcgctcagagcccctgccccccagtgccaccgctcccccaccgccgggccccatgcagccccgcctggagcagctcaaaactcacgt ccaggtgatcaagaggtcagccaagccgagtgagaagccccggctgcggcagataccctcggctgaagacctggagacagatggcgggg gaccgggccaggtggtggacgatggcctggagcacagggagctgggccatgggcagcctgaggccagaggccccgctcctctccagcag caccctcaggtgttgctctgggaacagcagcgactggctgggcggctcccccggggcagcaccggggacactgtgctgcttcctctggccca gggtgggcaccggcctctgtcccgggctcagtcttccccagccgcacctgcctcactgtcagccccagagcctgccagccaggcccgagtcct ctccagctcagagacccctgccaggaccctgcccttcaccacagggctgatctatgactcggtcatgctgaagcaccagtgctcctgcggtga caacagcaggcacccggagcacgccggccgcatccagagcatctggtcccggctgcaggagcgggggctccggagccagtgtgagtgtct ccgaggccggaaggcctccctggaagagctgcagtcggtccactctgagcggcacgtgctcctctacggcaccaacccgctcagccgcctca aactggacaacgggaagctggcagggctcctggcacagcggatgtttgtgatgctgccctgtggtggggttggggtggacactgacaccat ctggaatgagcttcattcctccaatgcagcccgctgggccgctggcagtgtcactgacctcgccttcaaagtggcttctcgtgagctaaagaat ggtttcgctgtggtgcggcccccaggacaccatgcagatcattcaacagccatgggcttctgcttcttcaactcagtggccatcgcctgccggc agctgcaacagcagagcaaggccagcaagatcctcattgtagactgggacgtgcaccatggcaacggcacccagcaaaccttctaccaag accccagtgtgctctacatctccctgcatcgccatgacgacggcaacttcttcccggggagtggggctgtggatgaggtaggggctggcagc ggtgagggcttcaatgtcaatgtggcctgggctggaggtctggacccccccatgggggatcctgagtacctggctgctttcaggatagtcgtg atgcccatcgcccgagagttctctccagacctagtcctggtgtctgctggatttgatgctgctgagggtcacccggccccactgggtggctacc atgtttctgccaaatgttttggatacatgacgcagcaactgatgaacctggcaggaggcgcagtggtgctggccttggagggtggccatgac ctcacagccatctgtgacgcctctgaggcctgtgtggctgctcttctgggtaacagggtggatcccctttcagaagaaggctggaaacagaaa cccaacctcaatgccatccgctctctggaggccgtgatccgggtgcacagtaaatactggggctgcatgcagcgcctggcctcctgtccagac tcctgggtgcctagagtgccaggggctgacaaagaagaagtggaggcagtgaccgcactggcgtccctctctgtgggcatcctggctgaag ataggccctcggagcagctggtggaggaggaagaacctatgaatctc [SEQ ID NO: 16], corresponding to the full-length human HDAC7 isoform A coding sequence set forth in GenBank Accession No. NM_015401;
atggacctgcgggtgggccagcggcccccagtggagcccccaccagagcccacattgctggccctgcagcgtcc ccagcgcctgcaccaccacctcttcctagcaggcctgcagcagcagcgctcggtggagcccatgaggctctccatggacacgccgatgcccg agttgcaggtgggaccccaggaacaagagctgcggcagcttctccacaaggacaagagcaagcgaagtgctgtagccagcagcgtggtc aagcagaagctagcggaggtgattctgaaaaaacagcaggcggccctagaaagaacagtccatcccaacagccccggcattccctacaga accctggagcccctggagacggaaggagccacccgctccatgctcagcagctttttgcctcctgttcccagcctgcccagtgaccccccagag cacttccctctgcgcaagacagtctctgagcccaacctgaagctgcgctataagcccaagaagtccctggagcggaggaagaatccactgct ccgaaaggagagtgcgccccccagcctccggcggcggcccgcagagaccctcggagactcctccccaagtagtagcagcacgcccgcatc agggtgcagctcccccaatgacagcgagcacggccccaatcccatcctgggctcggaggcgctcttgggccagcggctgcggctgcaggag acttctgtggccccgttcgccttgccgacagtgtccttgctgcccgcaatcactctggggctgcccgcccctgccagggctgacagtgaccgca ggacccatccgactctgggccctcgggggccaatcctggggagcccccacactcccctcttcctgccccatggcttggagcccgaggctgggg gcaccttgccctctcgcctgcagcccattctcctcctggacccctcaggctctcatgccccgctgctgactgtgcccgggcttgggcccttgccctt ccactttgcccagtccttaatgaccaccgagcggctctctgggtcaggcctccactggccactgagccggactcgctcagagcccctgcccccc agtgccaccgctcccccaccgccgggccccatgcagccccgcctggagcagctcaaaactcacgtccaggtgatcaagaggtcagccaagc cgagtgagaagccccggctgcggcagataccctcggctgaagacctggagacagatggcgggggaccgggccaggtggtggacgatgg cctggagcacagggagctgggccatgggcagcctgaggccagaggccccgctcctctccagcagcaccctcaggtgttgctctgggaacag cagcgactggctgggcggctcccccggggcagcaccggggacactgtgctgcttcctctggcccagggtgggcaccggcctctgtcccgggc tcagtcttccccagccgcacctgcctcactgtcagccccagagcctgccagccaggcccgagtcctctccagctcagagacccctgccaggac cctgcccttcaccacagggctgatctatgactcggtcatgctgaagcaccagtgctcctgcggtgacaacagcaggcacccggagcacgccg gccgcatccagagcatctggtcccggctgcaggagcgggggctccggagccagtgtgagtgtctccgaggccggaaggcctccctggaag agctgcagtcggtccactctgagcggcacgtgctcctctacggcaccaacccgctcagccgcctcaaactggacaacgggaagctggcagg gctcctggcacagcggatgtttgtgatgctgccctgtggtggggttggggtggacactgacaccatctggaatgagcttcattcctccaatgca gcccgctgggccgctggcagtgtcactgacctcgccttcaaagtggcttctcgtgagctaaagaatggtttcgctgtggtgcggcccccagga caccatgcagatcattcaacagccatgggcttctgcttcttcaactcagtggccatcgcctgccggcagctgcaacagcagagcaaggccagc aagatcctcattgtagactgggacgtgcaccatggcaacggcacccagcaaaccttctaccaagaccccagtgtgctctacatctccctgcatc gccatgacgacggcaacttcttcccggggagtggggctgtggatgaggtaggggctggcagcggtgagggcttcaatgtcaatgtggcctg ggctggaggtctggacccccccatgggggatcctgagtacctggctgctttcaggatagtcgtgatgcccatcgcccgagagttctctccaga cctagtcctggtgtctgctggatttgatgctgctgagggtcacccggccccactgggtggctaccatgtttctgccaaatgttttggatacatgac gcagcaactgatgaacctggcaggaggcgcagtggtgctggccttggagggtggccatgacctcacagccatctgtgacgcctctgaggcct gtgtggctgctcttctgggtaacagggtggatcccctttcagaagaaggctggaaacagaaacccaacctcaatgccatccgctctctggagg ccgtgatccgggtgcacagtaaatactggggctgcatgcagcgcctggcctcctgtccagactcctgggtgcctagagtgccaggggctgac aaagaagaagtggaggcagtgaccgcactggcgtccctctctgtgggcatcctggctgaagataggccctcggagcagctggtggaggag gaagaacctatgaatctc [SEQ ID NO: 17], corresponding to the portion of the coding sequence for the HDAC polypeptide of SEQ ID NO:9 that codes for the HDCA7 polypeptide set forth in SEQ ID NO:10;
atggacctgcgggtgggccagcggcccccagtggagcccccaccagagcccacattgctggccctgcagcgtcc ccagcgcctgcaccaccacctcttcctagcaggcctgcagcagcagcgctcggtggagcccatgaggctctccatggacacgccgatgcccg agttgcaggtgggaccccaggaacaagagctgcggcagcttctccacaaggacaagagcaagcgaagtgctgtagccagcagcgtggtc aagcagaagctagcggaggtgattctgaaaaaacagcaggcggccctagaaagaacagtccatcccaacagccccggcattccctacaga accctggagcccctggagacggaaggagccacccgctccatgctcagcagctttttgcctcctgttcccagcctgcccagtgaccccccagag cacttccctctgcgcaagacagtctctgagcccaacctgaagctgcgctataagcccaagaagtccctggagcggaggaagaatccactgct ccgaaaggagagtgcgccccccagcctccggcggcggcccgcagagaccctcggagactcctccccaagtagtagcagcacgcccgcatc agggtgcagctcccccaatgacagcgagcacggccccaatcccatcctgggctcggaggcgctcttgggccagcggctgcggctgcaggag acttctgtggccccgttcgccttgccgacagtgtccttgctgcccgcaatcactctggggctgcccgcccctgccagggctgacagtgaccgca ggacccatccgactctgggccctcgggggccaatcctggggagcccccacactcccctcttcctgccccatggcttggagcccgaggctgggg gcaccttgccctctcgcctgcagcccattctcctcctggacccctcaggctctcatgccccgctgctgactgtgcccgggcttgggcccttgccctt ccactttgcccagtccttaatgaccaccgagcggctctctgggtcaggcctccactggccactgagccggactcgctcagagcccctgcccccc agtgccaccgctcccccaccgccgggccccatgcagccccgcctggagcagctcaaaactcacgtccaggtgatcaagaggtcagccaagc cgagtgagaagccccggctgcggcagataccctcggctgaagacctggagacagatggcgggggaccgggccaggtggtggacgatgg cctggagcacagggagctgggccatgggcagcctgaggccagaggccccgctcctctccagcagcaccctcaggtgttgctctgggaacag cagcgactggctgggcggctcccccggggcagcaccggggacactgtgctgcttcctctggcccagggtgggcaccggcctctgtcccgggc tcagtcttccccagccgcacctgcctcactgtcagccccagagcctgccagccaggcccgagtcctctccagctcagagacc [SEQ ID NO: 18], corresponding to the portion of the coding sequence for the HDAC polypeptide of SEQ ID NO:9 that codes for the HDCA7 polypeptide set forth in SEQ ID NO:11;
atgcacagccccggcgcgggctgccctgccctccagccagacacaccaggctctcagccccaacccatggacct gcgggtgggccagcggcccacggtggagcccccaccagagcctgcgctgctgaccctgcaacacccccaacgcctgcaccgccatctcttcc tggcaggcttacaccagcaacagcgctcagccgagcccatgaggctctccatggacccaccaatgccggagctgcaggggggacagcagg agcaagaacttcggcaacttctcaataaagacaagagcaagcgaagtgccgtagccagcagtgtggtcaagcagaagctggctgaagtga tcctgaagaaacagcaggcagcccttgagagaacagtccatcccagcagccccagtattccctacagaactcttgagcccttggacacagag ggtgctgcccgctccgtgcttagcagcttcctgcctcctgttcccagcctgcccactgaacccccggaacactttcccttgcgtaaaacagtgtct gaacccaacctgaagttgcgctacaaacccaagaaatccctggagagacgcaagaatcccctgctcaggaaggagagtgccccgcccagc cttcggaggaggcctgccgagacccttggagattcctcccccagtagtagcagcacacccgcgtcagggtgcagctcccctaatgacagcga gcatggccctaaccctgccctaggctcagaggctgatggtgaccgcaggacccattcaactttaggccctcggggtcctgtactggggaaccc ccatgctcccctcttcctgcaccacggtctggagccagaggctgggggcaccttaccctctcgcctgcaacccattctcctgctggacccctcag tctctcatgccccactgtggactgtgcctggccttgggcccttgcccttccactttgcccagcccttactgaccaccgagcggctctctgggtcag gcctccatcgaccacttaaccggacccgctcagagcccctgccccccagcgccacagcctcccctctgctggcccccctgcagccccgccagg atcggctcaaacctcacgtccagctgatcaagccagccatctcccctccccagaggcctgccaagcccagtgagaagccccgactgcgacag ataccctcggctgaggacctagagacagatggtgggggagtgggacctatggcgaatgatggcctggaacatagggagtcaggccgtgg gcctcctgagggcagaggctccatttctctgcagcagcaccaacaggtgccaccctgggagcagcagcatctagccgggcggctctctcagg gaagccccggggactccgtgctgatacctctggcccaggttggacaccggcccctgtccagaacccagtcttccccagcagcacctgtttccat gttgagcccagagcccacctgtcagacccaagtcctcaacagctcagagacacctgctacagggctggtctatgactcggtgatgctgaaac accaatgttcctgtggagacaacagcaagcatcccgagcatgcaggccgcatccagagcatctggtcccggctgcaggaacggggtctccg cagccagtgtgagtgtctccgaggccgaaaggcttccctagaggagctgcagtcagtccactctgaacggcacgtgctcctttacggcacga acccactcagccgcctcaaactggataacgggaagcttacaggactcctggcacagcggacgtttgtgatgctaccctgtggcggggttggg gtcgatactgacaccatctggaacgagctgcattcctccaatgcagcccgctgggctgcgggcagtgtcaccgaccttgccttcaaagtagctt cccgagagctgaagaatggctttgctgtggtgcgacccccgggacaccatgcagatcattctacagccatgggcttctgcttcttcaactccgt ggccatcgcctgccgacagctacagcaacacggcaaagccagcaagatcctcattgttgactgggatgttcaccatggcaacggcacacag cagactttctaccaggaccccagtgtgctctacatttcccttcatcgccatgacgacggcaacttcttcccaggcagtggggccgtggatgagg tgggaactgccagtggcgagggcttcaatgtcaacgtggcttgggctgggggcttggatccacccatgggggatcctgagtacctggctgctt tcaggatagtggtgatgcccattgcccgagagtttgctccagacctggtcctggtgtctgctgggtttgatgctgcggagggtcacccagcccc actgggtggctaccatgtttctgccaaatgttttgggtacatgacgcagcagttgatgaacttggcaggaggcgccgtggtgttggccttagag ggtggacatgacctcacggccatctgtgatgcctcggaggcctgtgtagctgctcttctgggcaacaaggtggaccccctttcagaagaaagc tggaaacagaaacccaacctcagtgccatccgctcgctggaagctgtggtcagggtgcacaggaaatactggggctgcatgcagcgcttgg cctcctgtccagactcctggctacccagagtgccgggagctgatgcagaagtggaagccgtgaccgcgctggcatccctttctgtgggcatcc tggctgaagacaggccctcggagcggctggtggaagaggaagaacccatgaacctc [SEQ ID NO: 19], corresponding to the coding sequence for the full-length mouse HDAC7 polypeptide of SEQ ID NO:12, and to the coding sequence set out in NCBI Reference Sequence: NM_019572.3;
atggacctgcgggtgggccagcggcccacggtggagcccccaccagagcctgcgctgctgaccctgcaacaccc ccaacgcctgcaccgccatctcttcctggcaggcttacaccagcaacagcgctcagccgagcccatgaggctctccatggacccaccaatgcc ggagctgcaggggggacagcaggagcaagaacttcggcaacttctcaataaagacaagagcaagcgaagtgccgtagccagcagtgtgg tcaagcagaagctggctgaagtgatcctgaagaaacagcaggcagcccttgagagaacagtccatcccagcagccccagtattccctacag aactcttgagcccttggacacagagggtgctgcccgctccgtgcttagcagcttcctgcctcctgttcccagcctgcccactgaacccccggaa cactttcccttgcgtaaaacagtgtctgaacccaacctgaagttgcgctacaaacccaagaaatccctggagagacgcaagaatcccctgctc aggaaggagagtgccccgcccagccttcggaggaggcctgccgagacccttggagattcctcccccagtagtagcagcacacccgcgtcag ggtgcagctcccctaatgacagcgagcatggccctaaccctgccctaggctcagaggctgatggtgaccgcaggacccattcaactttaggc cctcggggtcctgtactggggaacccccatgctcccctcttcctgcaccacggtctggagccagaggctgggggcaccttaccctctcgcctgc aacccattctcctgctggacccctcagtctctcatgccccactgtggactgtgcctggccttgggcccttgcccttccactttgcccagcccttact gaccaccgagcggctctctgggtcaggcctccatcgaccacttaaccggacccgctcagagcccctgccccccagcgccacagcctcccctct gctggcccccctgcagccccgccaggatcggctcaaacctcacgtccagctgatcaagccagccatctcccctccccagaggcctgccaagcc cagtgagaagccccgactgcgacagataccctcggctgaggacctagagacagatggtgggggagtgggacctatggcgaatgatggcct ggaacatagggagtcaggccgtgggcctcctgagggcagaggctccatttctctgcagcagcaccaacaggtgccaccctgggagcagca gcatctagccgggcggctctctcagggaagccccggggactccgtgctgatacctctggcccaggttggacaccggcccctgtccagaaccc agtcttccccagcagcacctgtctccatgctgagcccagagcccacctgtcagacccaagtcctcaacagctcagagacacctgctacagggc tggtctatgactcggtgatgctgaaacaccaatgttcctgtggagacaacagcaagcatcccgagcatgcaggccgcatccagagcatctgg tcccggctgcaggaacggggtctccgcagccagtgtgagtgtctccgaggccgaaaggcttccctagaggagctgcagtcagtccactctga acggcacgtgctcctctacggcacgaacccactcagccgcctcaaactggataacgggaagcttacaggactcctggcacagcggacgtttg tgatgctaccctgtggcggggttggggtcgatactgacaccatctggaacgagctgcattcctccaatgcagcccgctgggctgcgggcagc gtcaccgaccttgccttcaaagtagcttcccgagagctgaagaacggctttgctgtggtgcgacccccgggacaccatgcagatcattctaca gccatgggcttctgcttcttcaactccgtggccatcgcctgccgacagctacagcaacacggcaaagccagcaagatcctcattgttgactggg atgttcaccatggcaacggcacacagcagactttctaccaggaccccagtgtgctctacatttcccttcatcgtcatgacgacggcaacttcttc ccaggcagtggggccgtggatgaggtgggaactggcagtggcgagggcttcaatgtcaacgtggcttgggctgggggcttggatccaccc atgggggatcctgagtacctggctgctttcaggatagtggtgatgcccattgcccgagagtttgctccagacctggtcctggtgtctgctgggtt tgatgctgcggagggtcacccagccccactgggtggctaccatgtttctgccaaatgttttgggtacatgacgcagcagttgatgaacttggca ggaggcgccgtggtgttggccttagagggtggacatgacctcacggccatctgtgatgcctcggaggcctgtgtagctgctcttctgggcaac aaggtggaccccctttcagaagaaagctggaaacagaaacccaacctcagtgccatccgctcgctggaagctgtggtcagggtgcacagga aatactggggctgcatgcagcgcttggcctcctgtccagactcctggctacccagagtgccgggagctgatgcagaagtggaagccgtgacc gcgctggcatccctttctgtgggcatcctggctgaagacaggccctcggagcggctggtggaagaggaagaacccatgaacctc [SEQ ID NO:20], corresponding to the portion of the coding sequence for the HDAC polypeptide of SEQ ID NO:12 that codes for the HDCA7 polypeptide set forth in SEQ ID NO:13;
atggacctgcgggtgggccagcggcccacggtggagcccccaccagagcctgcgctgctgaccctgcaacaccc ccaacgcctgcaccgccatctcttcctggcaggcttacaccagcaacagcgctcagccgagcccatgaggctctccatggacccaccaatgcc ggagctgcaggggggacagcaggagcaagaacttcggcaacttctcaataaagacaagagcaagcgaagtgccgtagccagcagtgtgg tcaagcagaagctggctgaagtgatcctgaagaaacagcaggcagcccttgagagaacagtccatcccagcagccccagtattccctacag aactcttgagcccttggacacagagggtgctgcccgctccgtgcttagcagcttcctgcctcctgttcccagcctgcccactgaacccccggaa cactttcccttgcgtaaaacagtgtctgaacccaacctgaagttgcgctacaaacccaagaaatccctggagagacgcaagaatcccctgctc aggaaggagagtgccccgcccagccttcggaggaggcctgccgagacccttggagattcctcccccagtagtagcagcacacccgcgtcag ggtgcagctcccctaatgacagcgagcatggccctaaccctgccctaggctcagaggctgatggtgaccgcaggacccattcaactttaggc cctcggggtcctgtactggggaacccccatgctcccctcttcctgcaccacggtctggagccagaggctgggggcaccttaccctctcgcctgc aacccattctcctgctggacccctcagtctctcatgccccactgtggactgtgcctggccttgggcccttgcccttccactttgcccagcccttact gaccaccgagcggctctctgggtcaggcctccatcgaccacttaaccggacccgctcagagcccctgccccccagcgccacagcctcccctct gctggcccccctgcagccccgccaggatcggctcaaacctcacgtccagctgatcaagccagccatctcccctccccagaggcctgccaagcc cagtgagaagccccgactgcgacagataccctcggctgaggacctagagacagatggtgggggagtgggacctatggcgaatgatggcct ggaacatagggagtcaggccgtgggcctcctgagggcagaggctccatttctctgcagcagcaccaacaggtgccaccctgggagcagca gcatctagccgggcggctctctcagggaagccccggggactccgtgctgatacctctggcccaggttggacaccggcccctgtccagaaccc agtcttccccagcagcacctgtctccatgctgagcccagagcccacctgtcagacccaagtcctcaacagctcagagacacctgctacagggc tggtctatgactcggtgatgctgaaacaccaatgttcctgtggagacaacagcaagcatcccgagcatgcaggccgcatccagagcatctgg tcccggctgcaggaacggggtctccgcagccagtgtgagtgtctccgaggccgaaaggcttccctagaggagctgcagtcagtccactctga acggcacgtgctcctctacggcacgaacccactcagccgcctcaaactggataacgggaagcttacaggactcctggcacagcggacgtttg tgatgctaccctgtggcggggttggggtcgatactgacaccatctggaacgagctgcattcctccaatgcagcccgctgggctgcgggcagc gtcaccgaccttgccttcaaagtagcttcccgagagctgaagaacggctttgctgtggtgcgacccccgggacacgctgcagatcattctaca gccatgggcttctgcttcttcaactccgtggccatcgcctgccgacagctacagcaacacggcaaagccagcaagatcctcattgttgactggg atgttcaccatggcaacggcacacagcagactttctaccaggaccccagtgtgctctacatttcccttcatcgtcatgacgacggcaacttcttc ccaggcagtggggccgtggatgaggtgggaactggcagtggcgagggcttcaatgtcaacgtggcttgggctgggggcttggatccaccc atgggggatcctgagtacctggctgctttcaggatagtggtgatgcccattgcccgagagtttgctccagacctggtcctggtgtctgctgggtt tgatgctgcggagggtcacccagccccactgggtggctaccatgtttctgccaaatgttttgggtacatgacgcagcagttgatgaacttggca ggaggcgccgtggtgttggccttagagggtggacatgacctcacggccatctgtgatgcctcggaggcctgtgtagctgctcttctgggcaac aaggtggaccccctttcagaagaaagctggaaacagaaacccaacctcagtgccatccgctcgctggaagctgtggtcagggtgcacagga aatactggggctgcatgcagcgcttggcctcctgtccagactcctggctacccagagtgccgggagctgatgcagaagtggaagccgtgacc gcgctggcatccctttctgtgggcatcctggctgaagacaggccctcggagcggctggtggaagaggaagaacccatgaacct [SEQ ID NO:21], corresponding to the portion of the coding sequence for the HDAC polypeptide of SEQ ID NO:12 that codes for the HDCA7 polypeptide set forth in SEQ ID NO:14;
atggacctgcgggtgggccagcggcccacggtggagcccccaccagagcctgcgctgctgaccctgcaacaccc ccaacgcctgcaccgccatctcttcctggcaggcttacaccagcaacagcgctcagccgagcccatgaggctctccatggacccaccaatgcc ggagctgcaggggggacagcaggagcaagaacttcggcaacttctcaataaagacaagagcaagcgaagtgccgtagccagcagtgtgg tcaagcagaagctggctgaagtgatcctgaagaaacagcaggcagcccttgagagaacagtccatcccagcagccccagtattccctacag aactcttgagcccttggacacagagggtgctgcccgctccgtgcttagcagcttcctgcctcctgttcccagcctgcccactgaacccccggaa cactttcccttgcgtaaaacagtgtctgaacccaacctgaagttgcgctacaaacccaagaaatccctggagagacgcaagaatcccctgctc aggaaggagagtgccccgcccagccttcggaggaggcctgccgagacccttggagattcctcccccagtagtagcagcacacccgcgtcag ggtgcagctcccctaatgacagcgagcatggccctaaccctgccctaggctcagaggctgatggtgaccgcaggacccattcaactttaggc cctcggggtcctgtactggggaacccccatgctcccctcttcctgcaccacggtctggagccagaggctgggggcaccttaccctctcgcctgc aacccattctcctgctggacccctcagtctctcatgccccactgtggactgtgcctggccttgggcccttgcccttccactttgcccagcccttact gaccaccgagcggctctctgggtcaggcctccatcgaccacttaaccggacccgctcagagcccctgccccccagcgccacagcctcccctct gctggcccccctgcagccccgccaggatcggctcaaacctcacgtccagctgatcaagccagccatctcccctccccagaggcctgccaagcc cagtgagaagccccgactgcgacagataccctcggctgaggacctagagacagatggtgggggagtgggacctatggcgaatgatggcct ggaacatagggagtcaggccgtgggcctcctgagggcagaggctccatttctctgcagcagcaccaacaggtgccaccctgggagcagca gcatctagccgggcggctctctcagggaagccccggggactccgtgctgatacctctggcccaggttggacaccggcccctgtccagaaccc agtcttccccagcagcacctgtctccatgctgagcccagagcccacctgtcagacccaagtcctcaacagctcagagaca [SEQ ID NO:22], corresponding to the portion of the coding sequence for the HDAC polypeptide of SEQ ID NO:12 that codes for the HDCA7 polypeptide set forth in SEQ ID NO:15; and
a nucleic acid sequence corresponding to any one of the nucleic acid sequences set forth in SEQ ID NO:16-22, or to a coding sequence for a domain or motif thereof (e.g., coding sequence for PHA03247 super family motif) representative examples of which display at least about 70, 71, 72, 73, 74, 75, 76, 77, 78, 79, 80, 81, 82, 83, 84, 85, 86, 97, 88, 89, 90, 91, 92, 93, 94, 95, 96, 97, 98 or 99% sequence identity to a reference nucleic acid sequence selected from any one of SEQ ID NO:16-22, or hybridize to a reference nucleic acid sequence selected from any one of SEQ ID NO:16-22 under at least medium stringency, at least high stringency or very high stringency conditions.

The HDAC7 polypeptide-encoding nucleic acid molecules may be in the form a ribonucleic acid (RNA), or deoxyribonucleic acid (DNA), for enhancing the activity of 6PGD in a chosen cell (e.g., an immune cell such as an APC).

In some embodiments, the nucleic acid molecules comprise an RNA, suitably a messenger RNA (mRNA) polynucleotide, comprising an open reading frame (ORF) encoding a HDAC7 polypeptide (i.e., a coding sequence for a HDAC7 polypeptide). In other embodiments, the nucleic acid molecules are in the form of a nucleic acid construct comprising a DNA polynucleotide that comprises an ORF encoding a HDAC7 polypeptide, in operable connection with a regulatory element that is suitably operable in a chosen cell (e.g., an immune cell such as an APC).

### Codon Optimization

The polynucleotides of the present disclosure may be codon optimized. Codon optimization methods are known in the art and may be used for optimizing expression of the polypeptides disclosed herein. Codon optimization, in some embodiments, may be used to match codon frequencies in target and host organisms to ensure proper folding; bias GC content to increase mRNA stability or reduce secondary structures; minimize tandem repeat codons or base runs that may impair gene construction or expression; customize transcriptional and translational control regions; insert or remove protein trafficking sequences; remove/add post translation modification sites in encoded protein (e.g., glycosylation sites); add, remove or shuffle protein domains; insert or delete restriction sites; modify ribosome binding sites and mRNA degradation sites; adjust translational rates to allow the various domains of the protein to fold properly; or to reduce or eliminate problem secondary structures within the polynucleotide. Codon optimization tools, algorithms and services are known in the art. Non-limiting examples include services from GeneArt (Life Technologies), DNA2.0 (Menlo Park Calif.) and/or proprietary methods. In some embodiments, the open reading frame (ORF) sequence is optimized using optimization algorithms. In some embodiments a codon optimized RNA may, for instance, be one in which the levels of G/C are enhanced. The G/C-content of nucleic acid molecules may influence the stability of the RNA. RNA having an increased amount of guanine (G) and/or cytosine (C) residues may be functionally more stable than nucleic acids containing a large amount of adenine (A) and thymine (T) or uracil (U) nucleotides. WO02/098443 discloses a pharmaceutical composition containing an mRNA stabilized by sequence modifications in the translated region. Due to the degeneracy of the genetic code, the modifications work by substituting existing codons for those that promote greater RNA stability without changing the resulting amino acid. The approach is limited to coding regions of the RNA.

### Flanking Regions

Features, which can be considered beneficial in some embodiments of the present disclosure, can be encoded by regions of the polynucleotide and such regions can be upstream (5′) or downstream (3′) to, or within, a region that encodes a polypeptide. These regions can be incorporated into the polynucleotide before and/or after sequence optimization of the protein encoding region or open reading frame (ORF). It is not required that a polynucleotide contain both a 5′ and 3′ flanking region. Examples of such features include, but are not limited to, untranslated regions (UTRs), Kozak sequences, an oligo(dT) sequence, and detectable tags and can include multiple cloning sites that can have Xbal recognition.

In some embodiments, a 5′ UTR and/or a 3′ UTR region can be provided as flanking regions. Multiple 5′ or 3′ UTRs can be included in the flanking regions and can be the same or of different sequences. Any portion of the flanking regions, including none, can be sequence-optimized and any can independently contain one or more different structural or chemical modifications, before and/or after sequence optimization.

Untranslated regions (UTRs) are nucleic acid sections of a polynucleotide before a start codon (5′UTR) and after a stop codon (3′UTR) that are not translated. In some embodiments, a polynucleotide (e.g., a ribonucleic acid (RNA), e.g., a messenger RNA (mRNA)) of the disclosure comprising an open reading frame (ORF) encoding an antigen polypeptide further comprises UTR (e.g., a 5′UTR or functional fragment thereof, a 3′UTR or functional fragment thereof, or a combination thereof).

Exemplary UTRs include, but are not limited to, one or more 5′UTR and/or 3′UTR derived from the nucleic acid sequence of: a globin, such as an α- or β-globin (e.g., a Xenopus, mouse, rabbit, or human globin); a strong Kozak translational initiation signal; a CYBA (e.g., human cytochrome b-245α polypeptide); an albumin (e.g., human albumin7); a HSD17B4 (hydroxysteroid (17-β) dehydrogenase); a virus (e.g., a tobacco etch virus (TEV), a Venezuelan equine encephalitis virus (VEEV), a Dengue virus, a cytomegalovirus (CMV) (e.g., CMV immediate early 1 (IE1)), a hepatitis virus (e.g., hepatitis B virus), a sindbis virus, or a PAV barley yellow dwarf virus); a heat shock protein (e.g., hsp70); a translation initiation factor (e.g., eIF4G); a glucose transporter (e.g., hGLUT1 (human glucose transporter 1)); an actin (e.g., human a or 3 actin); a GAPDH; a tubulin; a histone; a citric acid cycle enzyme; a topoisomerase (e.g., a 5′UTR of a TOP gene lacking the 5′ TOP motif (the oligopyrimidine tract)); a ribosomal protein Large 32 (L32); a ribosomal protein (e.g., human or mouse ribosomal protein, such as, for example, rps9); an ATP synthase (e.g., ATP5A1 or the [subunit of mitochondrial H+-ATP synthase); a growth hormone e (e.g., bovine (bGH) or human (hGH)); an elongation factor (e.g., elongation factor 1 α1 (EEF1A1)); a manganese superoxide dismutase (MnSOD); a myocyte enhancer factor 2A (MEF2A); a β-F1-ATPase, a creatine kinase, a myoglobin, a granulocyte-colony stimulating factor (G-CSF); a collagen (e.g., collagen type I, alpha 2 (Col1A2), collagen type I, alpha 1 (Col1A1), collagen type VI, alpha 2 (Col6A2), collagen type VI, alpha 1 (Col6A1)); a ribophorin (e.g., ribophorin I (RPNI)); a low density lipoprotein receptor-related protein (e.g., LRP1); a cardiotrophin-like cytokine factor (e.g., Nnt1); calreticulin (Calr); a procollagen-lysine, 2-oxoglutarate 5-dioxygenase 1 (Plod1); and a nucleobindin (e.g., Nucb1).

In some embodiments, the 5′UTR is selected from the group consisting of a β-globin 5′UTR; a 5′UTR containing a strong Kozak translational initiation signal; a cytochrome b-245 α polypeptide (CYBA) 5′UTR; a hydroxysteroid (17-3) dehydrogenase (HSD17B4) 5′UTR; a Tobacco etch virus (TEV) 5′UTR; a Venezuelan equine encephalitis virus (TEEV) 5′UTR; a 5′ proximal open reading frame of rubella virus (RV) RNA encoding nonstructural proteins; a Dengue virus (DEN) 5′UTR; a heat shock protein 70 (Hsp70) 5′UTR; a eIF4G 5′UTR; a GLUT1 5′UTR; functional fragments thereof and any combination thereof.

In some embodiments, the 3′UTR is selected from the group consisting of a β-globin 3′UTR; a CYBA 3′UTR; an albumin 3′UTR; a growth hormone (GH) 3′UTR; a VEEV 3′UTR; a hepatitis B virus (HBV) 3′UTR; α-globin 3′UTR; a DEN 3′UTR; a PAV barley yellow dwarf virus (BYDV-PAV) 3′UTR; an elongation factor 1 α1 (EEF1A1) 3′UTR; a manganese superoxide dismutase (MnSOD) 3′UTR; a β subunit of mitochondrial H(+)-ATP synthase (P-mRNA) 3′UTR; a GLUT1 3′UTR; a MEF2A 3′UTR; a β-F1-ATPase 3′UTR; functional fragments thereof and combinations thereof.

In some embodiments, the polynucleotide comprises multiple UTRs, e.g., a double, a triple or a quadruple 5′UTR or 3′UTR. For example, a double UTR comprises two copies of the same UTR either in series or substantially in series. For example, a double beta-globin 3′UTR can be used (see US2010/0129877, the contents of which are incorporated herein by reference in its entirety).

In some embodiments, the UTR can also include at least one translation enhancer polynucleotide, translation enhancer element, or translational enhancer elements (collectively, “TEE,” which refers to nucleic acid sequences that increase the amount of polypeptide or protein produced from a polynucleotide. As a non-limiting example, the TEE can be located between the transcription promoter and the start codon. In some embodiments, the 5′UTR comprises a TEE. The TEE may be a conserved element in a UTR that can promote translational activity of a nucleic acid such as, but not limited to, cap-dependent or cap-independent translation. In one non-limiting example, the TEE comprises the TEE sequence in the 5′-leader of the Gtx homeodomain protein. See Chappell et al., PNAS 2004 101:9590-9594.

In some embodiments, RNA polynucleotides of the present disclosure comprise a 5′UTR element, an optionally codon optimized open reading frame, and a 3′UTR element, a poly(A) sequence and/or a polyadenylation signal, wherein the RNA is not chemically modified.

The RNA polynucleotide may be transcribed in vitro from template DNA, referred to as an “in vitro transcription template”. In some embodiments, an in vitro transcription template encodes a 5′ untranslated (UTR) region, contains an open reading frame, and encodes a 3′ UTR and a polyA tail. The particular nucleotide sequence composition and length of an in vitro transcription template will depend on the mRNA encoded by the template.

### Chemical Modifications

In some embodiments, an polynucleotides of the present disclosure are chemically modified. As used herein in reference to a polynucleotide, the terms “chemical modification” or, as appropriate, “chemically modified” refer to modification with respect to adenosine (A), guanosine (G), uridine (U), or cytidine (C) ribo- or deoxyribonucleosides in one or more of their position, pattern, percent or population. Generally, herein, these terms are not intended to refer to the ribonucleotide modifications in naturally occurring 5′-terminal mRNA cap moieties.

In some embodiments, the polynucleotides of the present disclosure can have a uniform chemical modification of all or any of the same nucleoside type or a population of modifications produced by mere downward titration of the same starting modification in all or any of the same nucleoside type, or a measured percent of a chemical modification of all any of the same nucleoside type but with random incorporation, such as where all uridines are replaced by a uridine analog, e.g., pseudouridine or 5-methoxyuridine. In another embodiment, the polynucleotides can have a uniform chemical modification of two, three, or four of the same nucleoside type throughout the entire polynucleotide (such as all uridines and all cytosines, etc. are modified in the same way).

Modified nucleotide base pairing encompasses not only the standard adenosine-thymine, adenosine-uracil, or guanosine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures. One example of such non-standard base pairing is the base pairing between the modified nucleotide inosine and adenine, cytosine or uracil. Any combination of base/sugar or linker can be incorporated into polynucleotides of the present disclosure.

The skilled artisan will appreciate that, except where otherwise noted, polynucleotide sequences set forth in the instant application will recite “T”s in a representative DNA sequence but where the sequence represents RNA, the “T”s would be substituted for “U”s.

Modifications of polynucleotides (e.g., RNA polynucleotides, such as mRNA polynucleotides) that are useful in the agent, compositions and methods of the present disclosure include, but are not limited to the following nucleotides, nucleosides, and nucleobases: 2-methylthio-N6-(cis-hydroxyisopentenyl)adenosine; 2-methylthio-N6-methyladenosine; 2-methylthio-N6-threonyl carbamoyladenosine; N6-glycinylcarbamoyladenosine; N6-isopentenyladenosine; N6-methyladenosine; N6-threonyl carbamoyladenosine; 1,2′-O-dimethyladenosine; 1-methyladenosine; 2′-O-methyladenosine; 2′-O-ribosyladenosine (phosphate); 2-methyladenosine; 2-methylthio-N6 isopentenyladenosine; 2-methylthio-N6-hydroxynorvalyl carbamoyladenosine; 2′-O-methyladenosine; 2′-O-ribosyladenosine (phosphate); Isopentenyladenosine; N6-(cis-hydroxyisopentenyl)adenosine; N6,2′-O-dimethyladenosine; N6,2′-O-dimethyladenosine; N6,N6,2′-O-trimethyladenosine; N6,N6-dimethyladenosine; N6-acetyladenosine; N6-hydroxynorvalylcarbamoyladenosine; N6-methyl-N6-threonylcarbamoyladenosine; 2-methyladenosine; 2-methylthio-N6-isopentenyladenosine; 7-deaza-adenosine; N1-methyl-adenosine; N6, N6 (dimethyl)adenine; N6-cis-hydroxy-isopentenyl-adenosine; α-thio-adenosine; 2 (amino)adenine; 2 (aminopropyl)adenine; 2 (methylthio) N6 (isopentenyl)adenine; 2-(alkyl)adenine; 2-(aminoalkyl)adenine; 2-(aminopropyl)adenine; 2-(halo)adenine; 2-(halo)adenine; 2-(propyl) adenine; 2′-Amino-2′-deoxy-ATP; 2′-Azido-2′-deoxy-ATP; 2′-Deoxy-2′-a-aminoadenosine TP; 2′-Deoxy-2′-a-azidoadenosine TP; 6 (alkyl)adenine; 6 (methyl)adenine; 6-(alkyl)adenine; 6-(methyl)adenine; 7 (deaza)adenine; 8 (alkenyl)adenine; 8 (alkynyl)adenine; 8 (amino)adenine; 8 (thioalkyl)adenine; 8-(alkenyl)adenine; 8-(alkyl)adenine; 8-(alkynyl)adenine; 8-(amino)adenine; 8-(halo)adenine; 8-(hydroxyl)adenine; 8-(thioalkyl)adenine; 8-(thiol)adenine; 8-azido-adeno sine; aza adenine; deaza adenine; N6 (methyl)adenine; N6-(isopentyl)adenine; 7-deaza-8-aza-adenosine; 7-methyladenine; 1-Deazaadenosine TP; 2′Fluoro-N6-Bz-deoxyadenosine TP; 2′-OMe-2-Amino-ATP; 2′O-methyl-N6-Bz-deoxyadenosine TP; 2′-a-Ethynyladenosine TP; 2-aminoadenine; 2-Aminoadenosine TP; 2-Amino-ATP; 2′-a-Trifluoromethyladenosine TP; 2-Azidoadenosine TP; 2′-b-Ethynyladenosine TP; 2-Bromoadenosine TP; 2′-b-Trifluoromethyladenosine TP; 2-Chloroadenosine TP; 2′-Deoxy-2′,2′-difluoroadenosine TP; 2′-Deoxy-2′-a-mercaptoadenosine TP; 2′-Deoxy-2′-a-thiomethoxyadenosine TP; 2′-Deoxy-2′-b-aminoadenosine TP; 2′-Deoxy-2′-b-azidoadenosine TP; 2′-Deoxy-2′-b-bromoadenosine TP; 2′-Deoxy-2′-b-chloroadenosine TP; 2′-Deoxy-2′-b-fluoroadenosine TP; 2′-Deoxy-2′-b-iodoadenosine TP; 2′-Deoxy-2′-b-mercaptoadenosine TP; 2′-Deoxy-2′-b-thiomethoxyadenosine TP; 2-Fluoroadenosine TP; 2-Iodoadenosine TP; 2-Mercaptoadenosine TP; 2-methoxy-adenine; 2-methylthio-adenine; 2-Trifluoromethyladenosine TP; 3-Deaza-3-bromoadenosine TP; 3-Deaza-3-chloroadenosine TP; 3-Deaza-3-fluoroadenosine TP; 3-Deaza-3-iodoadenosine TP; 3-Deazaadenosine TP; 4′-Azidoadenosine TP; 4′-Carbocyclic adenosine TP; 4′-Ethynyladenosine TP; 5′-Homo-adenosine TP; 8-Aza-ATP; 8-bromo-adenosine TP; 8-Trifluoromethyladenosine TP; 9-Deazaadenosine TP; 2-aminopurine; 7-deaza-2,6-diaminopurine; 7-deaza-8-aza-2,6-diaminopurine; 7-deaza-8-aza-2-aminopurine; 2,6-diaminopurine; 7-deaza-8-aza-adenine, 7-deaza-2-aminopurine; 2-thiocytidine; 3-methylcytidine; 5-formylcytidine; 5-hydroxymethylcytidine; 5-methylcytidine; N4-acetylcytidine; 2′-O-methylcytidine; 2′-O-methylcytidine; 5,2′-O-dimethylcytidine; 5-formyl-2′-O-methylcytidine; Lysidine; N4,2′-O-dimethylcytidine; N4-acetyl-2′-O-methylcytidine; N4-methylcytidine; N4,N4-Dimethyl-2′-OMe-Cytidine TP; 4-methylcytidine; 5-aza-cytidine; Pseudo-iso-cytidine; pyrrolo-cytidine; α-thio-cytidine; 2-(thio)cytosine; 2′-Amino-2′-deoxy-CTP; 2′-Azido-2′-deoxy-CTP; 2′-Deoxy-2′-a-aminocytidine TP; 2′-Deoxy-2′-a-azidocytidine TP; 3 (deaza) 5 (aza)cytosine; 3 (methyl)cytosine; 3-(alkyl)cytosine; 3-(deaza) 5 (aza)cytosine; 3-(methyl)cytidine; 4,2′-O-dimethylcytidine; 5 (halo)cytosine; 5 (methyl)cytosine; 5 (propynyl)cytosine; 5 (trifluoromethyl)cytosine; 5-(alkyl)cytosine; 5-(alkynyl)cytosine; 5-(halo)cytosine; 5-(propynyl)cytosine; 5-(trifluoromethyl)cytosine; 5-bromo-cytidine; 5-iodo-cytidine; 5-propynyl cytosine; 6-(azo)cytosine; 6-aza-cytidine; aza cytosine; deaza cytosine; N4 (acetyl)cytosine; 1-methyl-1-deaza-pseudoisocytidine; 1-methyl-pseudoisocytidine; 2-methoxy-5-methyl-cytidine; 2-methoxy-cytidine; 2-thio-5-methyl-cytidine; 4-methoxy-1-methyl-pseudoisocytidine; 4-methoxy-pseudoisocytidine; 4-thio-1-methyl-1-deaza-pseudoisocytidine; 4-thio-1-methyl-pseudoisocytidine; 4-thio-pseudoisocytidine; 5-aza-zebularine; 5-methyl-zebularine; pyrrolo-pseudoisocytidine; Zebularine; (E)-5-(2-Bromo-vinyl)cytidine TP; 2,2′-anhydro-cytidine TP hydrochloride; 2′Fluor-N4-Bz-cytidine TP; 2′Fluoro-N4-Acetyl-cytidine TP; 2′-O-Methyl-N4-Acetyl-cytidine TP; 2′O-methyl-N4-Bz-cytidine TP; 2′-a-Ethynylcytidine TP; 2′-a-Trifluoromethylcytidine TP; 2′-b-Ethynylcytidine TP; 2′-b-Trifluoromethylcytidine TP; 2′-Deoxy-2′,2′-difluorocytidine TP; 2′-Deoxy-2′-a-mercaptocytidine TP; 2′-Deoxy-2′-a-thiomethoxycytidine TP; 2′-Deoxy-2′-b-aminocytidine TP; 2′-Deoxy-2′-b-azidocytidine TP; 2′-Deoxy-2′-b-bromocytidine TP; 2′-Deoxy-2′-b-chlorocytidine TP; 2′-Deoxy-2′-b-fluorocytidine TP; 2′-Deoxy-2′-b-iodocytidine TP; 2′-Deoxy-2′-b-mercaptocytidine TP; 2′-Deoxy-2′-b-thiomethoxycytidine TP; 2′-O-Methyl-5-(1-propynyl)cytidine TP; 3′-Ethynylcytidine TP; 4′-Azidocytidine TP; 4′-Carbocyclic cytidine TP; 4′-Ethynylcytidine TP; 5-(1-Propynyl)ara-cytidine TP; 5-(2-Chloro-phenyl)-2-thiocytidine TP; 5-(4-Amino-phenyl)-2-thiocytidine TP; 5-Aminoallyl-CTP; 5-Cyanocytidine TP; 5-Ethynylara-cytidine TP; 5-Ethynylcytidine TP; 5′-Homo-cytidine TP; 5-Methoxycytidine TP; 5-Trifluoromethyl-Cytidine TP; N4-Amino-cytidine TP; N4-Benzoyl-cytidine TP; Pseudoisocytidine; 7-methylguanosine; N2,2′-O-dimethylguanosine; N2-methylguanosine; Wyosine; 1,2′-O-dimethylguanosine; 1-methylguanosine; 2′-O-methylguanosine; 2′-O-ribosylguanosine (phosphate); 2′-O-methylguanosine; 2′-O-ribosylguanosine (phosphate); 7-aminomethyl-7-deazaguanosine; 7-cyano-7-deazaguanosine; Archaeosine; Methylwyo sine; N2,7-dimethylguanosine; N2,N2,2′-O-trimethylguanosine; N2,N2,7-trimethylguanosine; N2,N2-dimethylguanosine; N2,7,2′-O-trimethylguanosine; 6-thio-guanosine; 7-deaza-guanosine; 8-oxo-guanosine; N1-methyl-guanosine; α-thio-guanosine; 2 (propyl)guanine; 2-(alkyl)guanine; 2′-Amino-2′-deoxy-GTP; 2′-Azido-2′-deoxy-GTP; 2′-Deoxy-2′-a-aminoguanosine TP; 2′-Deoxy-2′-a-azidoguanosine TP; 6 (methyl)guanine; 6-(alkyl)guanine; 6-(methyl)guanine; 6-methyl-guanosine; 7 (alkyl)guanine; 7 (deaza)guanine; 7 (methyl)guanine; 7-(alkyl)guanine; 7-(deaza)guanine; 7-(methyl)guanine; 8 (alkyl)guanine; 8 (alkynyl)guanine; 8 (halo)guanine; 8 (thioalkyl)guanine; 8-(alkenyl)guanine; 8-(alkyl)guanine; 8-(alkynyl)guanine; 8-(amino)guanine; 8-(halo)guanine; 8-(hydroxyl)guanine; 8-(thioalkyl)guanine; 8-(thiol)guanine; aza guanine; deaza guanine; N (methyl)guanine; N-(methyl)guanine; 1-methyl-6-thio-guanosine; 6-methoxy-guanosine; 6-thio-7-deaza-8-aza-guanosine; 6-thio-7-deaza-guanosine; 6-thio-7-methyl-guanosine; 7-deaza-8-aza-guanosine; 7-methyl-8-oxo-guanosine; N2,N2-dimethyl-6-thio-guanosine; N2-methyl-6-thio-guanosine; 1-Me-GTP; 2′Fluoro-N2-isobutyl-guanosine TP; 2′O-methyl-N2-isobutyl-guanosine TP; 2′-a-Ethynylguanosine TP; 2′-a-Trifluoromethylguanosine TP; 2′-b-Ethynylguanosine TP; 2′-b-Trifluoromethylguanosine TP; 2′-Deoxy-2′,2′-difluoroguanosine TP; 2′-Deoxy-2′-a-mercaptoguanosine TP; 2′-Deoxy-2′-a-thiomethoxyguanosine TP; 2′-Deoxy-2′-b-aminoguanosine TP; 2′-Deoxy-2′-b-azidoguanosine TP; 2′-Deoxy-2′-b-bromoguanosine TP; 2′-Deoxy-2′-b-chloroguanosine TP; 2′-Deoxy-2′-b-fluoroguanosine TP; 2′-Deoxy-2′-b-iodoguanosine TP; 2′-Deoxy-2′-b-mercaptoguanosine TP; 2′-Deoxy-2′-b-thiomethoxyguanosine TP; 4′-Azidoguanosine TP; 4′-Carbocyclic guanosine TP; 4′-Ethynylguanosine TP; 5′-Homo-guanosine TP; 8-bromo-guanosine TP; 9-Deazaguanosine TP; N2-isobutyl-guanosine TP; 1-methylinosine; Inosine; 1,2′-O-dimethylinosine; 2′-O-methylinosine; 7-methylinosine; 2′-O-methylinosine; Epoxyqueuosine; galactosyl-queuosine; Mannosylqueuosine; Queuosine; allyamino-thymidine; aza thymidine; deaza thymidine; deoxy-thymidine; 2′-O-methyluridine; 2-thiouridine; 3-methyluridine; 5-carboxymethyluridine; 5-hydroxyuridine; 5-methyluridine; 5-taurinomethyl-2-thiouridine; 5-taurinomethyluridine; Dihydrouridine; Pseudouridine; (3-(3-amino-3-carboxypropyl)uridine; 1-methyl-3-(3-amino-5-carboxypropyl)pseudouridine; 1-methylpseduouridine; 1-ethyl-pseudouridine; 2′-O-methyluridine; 2′-O-methylpseudouridine; 2′-O-methyluridine; 2-thio-2′-O-methyluridine; 3-(3-amino-3-carboxypropyl)uridine; 3,2′-O-dimethyluridine; 3-Methyl-pseudo-Uridine TP; 4-thiouridine; 5-(carboxyhydroxymethyl)uridine; 5-(carboxyhydroxymethyl)uridine methyl ester; 5,2′-O-dimethyluridine; 5,6-dihydro-uridine; 5-aminomethyl-2-thiouridine; 5-carbamoylmethyl-2′-O-methyluridine; 5-carbamoylmethyluridine; 5-carboxyhydroxymethyluridine; 5-carboxyhydroxymethyluridine methyl ester; 5-carboxymethylaminomethyl-2′-O-methyluridine; 5-carboxymethylaminomethyl-2-thiouridine; 5-carboxymethylaminomethyl-2-thiouridine; 5-carboxymethylaminomethyluridine; 5-carboxymethylaminomethyluridine; 5-Carbamoylmethyluridine TP; 5-methoxycarbonylmethyl-2′-O-methyluridine; 5-methoxycarbonylmethyl-2-thiouridine; 5-methoxycarbonylmethyluridine; 5-methyluridine,), 5-methoxyuridine; 5-methyl-2-thiouridine; 5-methylaminomethyl-2-selenouridine; 5-methylaminomethyl-2-thiouridine; 5-methylaminomethyluridine; 5-Methyldihydrouridine; 5-Oxyacetic acid-Uridine TP; 5-Oxyacetic acid-methyl ester-Uridine TP; N1-methyl-pseudo-uracil; N1-ethyl-pseudo-uracil; uridine 5-oxyacetic acid; uridine 5-oxyacetic acid methyl ester; 3-(3-Amino-3-carboxypropyl)-Uridine TP; 5-(iso-Pentenylaminomethyl)-2-thiouridine TP; 5-(iso-Pentenylaminomethyl)-2′-O-methyluridine TP; 5-(iso-Pentenylaminomethyl)uridine TP; 5-propynyl uracil; α-thio-uridine; 1 (aminoalkylamino-carbonylethylenyl)-2(thio)-pseudouracil; 1 (aminoalkylaminocarbonylethylenyl)-2,4-(dithio)pseudouracil; 1 (aminoalkylaminocarbonylethylenyl)-4 (thio)pseudouracil; 1 (aminoalkylaminocarbonylethylenyl)-pseudouracil; 1 (aminocarbonylethylenyl)-2(thio)-pseudouracil; 1 (aminocarbonylethylenyl)-2,4-(dithio)pseudouracil; 1 (aminocarbonylethylenyl)-4 (thio)pseudouracil; 1 (aminocarbonylethylenyl)-pseudouracil; 1 substituted 2(thio)-pseudouracil; 1 substituted 2,4-(dithio)pseudouracil; 1 substituted 4 (thio)pseudouracil; 1 substituted pseudouracil; 1-(aminoalkylamino-carbonylethylenyl)-2-(thio)-pseudouracil; 1-Methyl-3-(3-amino-3-carboxypropyl) pseudouridine TP; 1-Methyl-3-(3-amino-3-carboxypropyl)pseudo-UTP; 1-Methyl-pseudo-UTP; 1-Ethyl-pseudo-UTP; 2 (thio)pseudouracil; 2′ deoxy uridine; 2′ fluorouridine; 2-(thio)uracil; 2,4-(dithio)psuedouracil; 2′ methyl, 2′amino, 2′azido, 2′fluro-guanosine; 2′-Amino-2′-deoxy-UTP; 2′-Azido-2′-deoxy-UTP; 2′-Azido-deoxyuridine TP; 2′-O-methylpseudouridine; 2′ deoxy uridine; 2′ fluorouridine; 2′-Deoxy-2′-a-aminouridine TP; 2′-Deoxy-2′-a-azidouridine TP; 2-methylpseudouridine; 3 (3 amino-3 carboxypropyl)uracil; 4 (thio)pseudouracil; 4-(thio) pseudouracil; 4-(thio)uracil; 4-thiouracil; 5 (1,3-diazole-1-alkyl)uracil; 5 (2-aminopropyl)uracil; 5 (aminoalkyl)uracil; 5 (dimethylaminoalkyl)uracil; 5 (guanidiniumalkyl)uracil; 5 (methoxycarbonylmethyl)-2-(thio)uracil; 5 (methoxycarbonyl-methyl)uracil; 5 (methyl) 2 (thio)uracil; 5 (methyl) 2,4 (dithio)uracil; 5 (methyl) 4 (thio)uracil; 5 (methylaminomethyl)-2 (thio)uracil; 5 (methylaminomethyl)-2,4 (dithio)uracil; 5 (methylaminomethyl)-4 (thio)uracil; 5 (propynyl)uracil; 5 (trifluoromethyl)uracil; 5-(2-aminopropyl)uracil; 5-(alkyl)-2-(thio)pseudouracil; 5-(alkyl)-2,4 (dithio)pseudouracil; 5-(alkyl)-4 (thio)pseudouracil; 5-(alkyl)pseudouracil; 5-(alkyl)uracil; 5-(alkynyl)uracil; 5-(allylamino)uracil; 5-(cyanoalkyl)uracil; 5-(dialkylaminoalkyl)uracil; 5-(dimethylaminoalkyl)uracil; 5-(guanidiniumalkyl)uracil; 5-(halo)uracil; 5-(1,3-diazole-1-alkyl)uracil; 5-(methoxy)uracil; 5-(methoxycarbonylmethyl)-2-(thio)uracil; 5-(methoxycarbonyl-methyl)uracil; 5-(methyl) 2(thio)uracil; 5-(methyl) 2,4 (dithio)uracil; 5-(methyl) 4 (thio)uracil; 5-(methyl)-2-(thio)pseudouracil; 5-(methyl)-2,4 (dithio)pseudouracil; 5-(methyl)-4 (thio)pseudouracil; 5-(methyl)pseudouracil; 5-(methylaminomethyl)-2 (thio)uracil; 5-(methylaminomethyl)-2,4(dithio)uracil; 5-(methylaminomethyl)-4-(thio)uracil; 5-(propynyl)uracil; 5-(trifluoromethyl)uracil; 5-aminoallyl-uridine; 5-bromo-uridine; 5-iodo-uridine; 5-uracil; 6 (azo)uracil; 6-(azo)uracil; 6-aza-uridine; allyamino-uracil; aza uracil; deaza uracil; N3 (methyl)uracil; Pseudo-UTP-1-2-ethanoic acid; Pseudouracil; 4-Thio-pseudo-UTP; 1-carboxymethyl-pseudouridine; 1-methyl-1-deaza-pseudouridine; 1-propynyl-uridine; 1-taurinomethyl-1-methyl-uridine; 1-taurinomethyl-4-thio-uridine; 1-taurinomethyl-pseudouridine; 2-methoxy-4-thio-pseudouridine; 2-thio-1-methyl-1-deaza-pseudouridine; 2-thio-1-methyl-pseudouridine; 2-thio-5-aza-uridine; 2-thio-dihydropseudouridine; 2-thio-dihydrouridine; 2-thio-pseudouridine; 4-methoxy-2-thio-pseudouridine; 4-methoxy-pseudouridine; 4-thio-1-methyl-pseudouridine; 4-thio-pseudouridine; 5-aza-uridine; Dihydropseudouridine; (±)1-(2-Hydroxypropyl)pseudouridine TP; (2R)-1-(2-Hydroxypropyl)pseudouridine TP; (2S)-1-(2-Hydroxypropyl)pseudouridine TP; (E)-5-(2-Bromo-vinyl)ara-uridine TP; (E)-5-(2-Bromo-vinyl)uridine TP; (Z)-5-(2-Bromo-vinyl)ara-uridine TP; (Z)-5-(2-Bromo-vinyl)uridine TP; 1-(2,2,2-Trifluoroethyl)-pseudo-UTP; 1-(2,2,3,3,3-Pentafluoropropyl)pseudouridine TP; 1-(2,2-Diethoxyethyl)pseudouridine TP; 1-(2,4,6-Trimethylbenzyl)pseudouridine TP; 1-(2,4,6-Trimethyl-benzyl)pseudo-UTP; 1-(2,4,6-Trimethyl-phenyl)pseudo-UTP; 1-(2-Amino-2-carboxyethyl)pseudo-UTP; 1-(2-Amino-ethyl)pseudo-UTP; 1-(2-Hydroxyethyl)pseudouridine TP; 1-(2-Methoxyethyl)pseudouridine TP; 1-(3,4-Bis-trifluoromethoxybenzyl)pseudouridine TP; 1-(3,4-Dimethoxybenzyl)pseudouridine TP; 1-(3-Amino-3-carboxypropyl)pseudo-UTP; 1-(3-Amino-propyl)pseudo-UTP; 1-(3-Cyclopropyl-prop-2-ynyl)pseudouridine TP; 1-(4-Amino-4-carboxybutyl)pseudo-UTP; 1-(4-Amino-benzyl)pseudo-UTP; 1-(4-Amino-butyl)pseudo-UTP; 1-(4-Amino-phenyl)pseudo-UTP; 1-(4-Azidobenzyl)pseudouridine TP; 1-(4-Bromobenzyl)pseudouridine TP; 1-(4-Chlorobenzyl)pseudouridine TP; 1-(4-Fluorobenzyl)pseudouridine TP; 1-(4-Iodobenzyl)pseudouridine TP; 1-(4-Methanesulfonylbenzyl)pseudouridine TP; 1-(4-Methoxybenzyl)pseudouridine TP; 1-(4-Methoxy-benzyl)pseudo-UTP; 1-(4-Methoxy-phenyl)pseudo-UTP; 1-(4-Methylbenzyl)pseudouridine TP; 1-(4-Methyl-benzyl)pseudo-UTP; 1-(4-Nitrobenzyl)pseudouridine TP; 1-(4-Nitro-benzyl)pseudo-UTP; 1(4-Nitro-phenyl)pseudo-UTP; 1-(4-Thiomethoxybenzyl)pseudouridine TP; 1-(4-Trifluoromethoxybenzyl)pseudouridine TP; 1-(4-Trifluoromethylbenzyl)pseudouridine TP; 1-(5-Amino-pentyl)pseudo-UTP; 1-(6-Amino-hexyl)pseudo-UTP; 1,6-Dimethyl-pseudo-UTP; 1-[3-(2-{2-[2-(2-Aminoethoxy)-ethoxy]-ethoxy}-ethoxy)-propionyl]pseudouridine TP; 1-{3-[2-(2-Aminoethoxy)-ethoxy]-propionyl}pseudouridine TP; 1-Acetylpseudouridine TP; 1-Alkyl-6-(1-propynyl)-pseudo-UTP; 1-Alkyl-6-(2-propynyl)-pseudo-UTP; 1-Alkyl-6-allyl-pseudo-UTP; 1-Alkyl-6-ethynyl-pseudo-UTP; 1-Alkyl-6-homoallyl-pseudo-UTP; 1-Alkyl-6-vinyl-pseudo-UTP; 1-Allylpseudouridine TP; 1-Aminomethyl-pseudo-UTP; 1-Benzoylpseudouridine TP; 1-Benzyloxymethylpseudouridine TP; 1-Benzyl-pseudo-UTP; 1-Biotinyl-PEG2-pseudouridine TP; 1-Biotinylpseudouridine TP; 1-Butyl-pseudo-UTP; 1-Cyanomethylpseudouridine TP; 1-Cyclobutylmethyl-pseudo-UTP; 1-Cyclobutyl-pseudo-UTP; 1-Cycloheptylmethyl-pseudo-UTP; 1-Cycloheptyl-pseudo-UTP; 1-Cyclohexylmethyl-pseudo-UTP; 1-Cyclohexyl-pseudo-UTP; 1-Cyclooctylmethyl-pseudo-UTP; 1-Cyclooctyl-pseudo-UTP; 1-Cyclopentylmethyl-pseudo-UTP; 1-Cyclopentyl-pseudo-UTP; 1-Cyclopropylmethyl-pseudo-UTP; 1-Cyclopropyl-pseudo-UTP; 1-Ethyl-pseudo-UTP; 1-Hexyl-pseudo-UTP; 1-Homoallylpseudouridine TP; 1-Hydroxymethylpseudouridine TP; 1-iso-propyl-pseudo-UTP; 1-Me-2-thio-pseudo-UTP; 1-Me-4-thio-pseudo-UTP; 1-Me-alpha-thio-pseudo-UTP; 1-Methanesulfonylmethylpseudouridine TP; 1-Methoxymethylpseudouridine TP; 1-Methyl-6-(2,2,2-Trifluoroethyl)pseudo-UTP; 1-Methyl-6-(4-morpholino)-pseudo-UTP; 1-Methyl-6-(4-thiomorpholino)-pseudo-UTP; 1-Methyl-6-(substituted phenyl) pseudo-UTP; 1-Methyl-6-amino-pseudo-UTP; 1-Methyl-6-azido-pseudo-UTP; 1-Methyl-6-bromo-pseudo-UTP; 1-Methyl-6-butyl-pseudo-UTP; 1-Methyl-6-chloro-pseudo-UTP; 1-Methyl-6-cyano-pseudo-UTP; 1-Methyl-6-dimethylamino-pseudo-UTP; 1-Methyl-6-ethoxy-pseudo-UTP; 1-Methyl-6-ethylcarboxylate-pseudo-UTP; 1-Methyl-6-ethyl-pseudo-UTP; 1-Methyl-6-fluoro-pseudo-UTP; 1-Methyl-6-formyl-pseudo-UTP; 1-Methyl-6-hydroxyamino-pseudo-UTP; 1-Methyl-6-hydroxy-pseudo-UTP; 1-Methyl-6-iodo-pseudo-UTP; 1-Methyl-6-iso-propyl-pseudo-UTP; 1-Methyl-6-methoxy-pseudo-UTP; 1-Methyl-6-methylamino-pseudo-UTP; 1-Methyl-6-phenyl-pseudo-UTP; 1-Methyl-6-propyl-pseudo-UTP; 1-Methyl-6-tert-butyl-pseudo-UTP; 1-Methyl-6-trifluoromethoxy-pseudo-UTP; 1-Methyl-6-trifluoromethyl-pseudo-UTP; 1-Morpholinomethylpseudouridine TP; 1-Pentyl-pseudo-UTP; 1-Phenyl-pseudo-UTP; 1-Pivaloylpseudouridine TP; 1-Propargylpseudouridine TP; 1-Propyl-pseudo-UTP; 1-propynyl-pseudouridine; 1-p-tolyl-pseudo-UTP; 1-tert-Butyl-pseudo-UTP; 1-Thiomethoxymethylpseudouridine TP; 1-Thiomorpholinomethylpseudouridine TP; 1-Trifluoroacetylpseudouridine TP; 1-Trifluoromethyl-pseudo-UTP; 1-Vinylpseudouridine TP; 2,2′-anhydro-uridine TP; 2′-bromo-deoxyuridine TP; 2′-F-5-Methyl-2′-deoxy-UTP; 2′-OMe-5-Me-UTP; 2′-OMe-pseudo-UTP; 2′-a-Ethynyluridine TP; 2′-a-Trifluoromethyluridine TP; 2′-b-Ethynyluridine TP; 2′-b-Trifluoromethyluridine TP; 2′-Deoxy-2′,2′-difluorouridine TP; 2′-Deoxy-2′-a-mercaptouridine TP; 2′-Deoxy-2′-a-thiomethoxyuridine TP; 2′-Deoxy-2′-b-aminouridine TP; 2′-Deoxy-2′-b-azidouridine TP; 2′-Deoxy-2′-b-bromouridine TP; 2′-Deoxy-2′-b-chlorouridine TP; 2′-Deoxy-2′-b-fluorouridine TP; 2′-Deoxy-2′-b-iodouridine TP; 2′-Deoxy-2′-b-mercaptouridine TP; 2′-Deoxy-2′-b-thiomethoxyuridine TP; 2-methoxy-4-thio-uridine; 2-methoxyuridine; 2′-O-Methyl-5-(1-propynyl)uridine TP; 3-Alkyl-pseudo-UTP; 4′-Azidouridine TP; 4′-Carbocyclic uridine TP; 4′-Ethynyluridine TP; 5-(1-Propynyl)ara-uridine TP; 5-(2-Furanyl)uridine TP; 5-Cyanouridine TP; 5-Dimethylaminouridine TP; 5′-Homo-uridine TP; 5-iodo-2′-fluoro-deoxyuridine TP; 5-Phenylethynyluridine TP; 5-Trideuteromethyl-6-deuterouridine TP; 5-Trifluoromethyl-Uridine TP; 5-Vinylarauridine TP; 6-(2,2,2-Trifluoroethyl)-pseudo-UTP; 6-(4-Morpholino)-pseudo-UTP; 6-(4-Thiomorpholino)-pseudo-UTP; 6-(Substituted-Phenyl)-pseudo-UTP; 6-Amino-pseudo-UTP; 6-Azido-pseudo-UTP; 6-Bromo-pseudo-UTP; 6-Butyl-pseudo-UTP; 6-Chloro-pseudo-UTP; 6-Cyano-pseudo-UTP; 6-Dimethylamino-pseudo-UTP; 6-Ethoxy-pseudo-UTP; 6-Ethylcarboxylate-pseudo-UTP; 6-Ethyl-pseudo-UTP; 6-Fluoro-pseudo-UTP; 6-Formyl-pseudo-UTP; 6-Hydroxyamino-pseudo-UTP; 6-Hydroxy-pseudo-UTP; 6-Iodo-pseudo-UTP; 6-iso-Propyl-pseudo-UTP; 6-Methoxy-pseudo-UTP; 6-Methylamino-pseudo-UTP; 6-Methyl-pseudo-UTP; 6-Phenyl-pseudo-UTP; 6-Phenyl-pseudo-UTP; 6-Propyl-pseudo-UTP; 6-tert-Butyl-pseudo-UTP; 6-Trifluoromethoxy-pseudo-UTP; 6-Trifluoromethyl-pseudo-UTP; Alpha-thio-pseudo-UTP; Pseudouridine 1-(4-methylbenzenesulfonic acid) TP; Pseudouridine 1-(4-methylbenzoic acid) TP; Pseudouridine TP 1-[3-(2-ethoxy)]propionic acid; Pseudouridine TP 1-[3-{2-(2-[2-(2-ethoxy)-ethoxy]-ethoxy)-ethoxy}]propionic acid; Pseudouridine TP 1-[3-{2-(2-[2-{2(2-ethoxy)-ethoxy}-ethoxy]-ethoxy)-ethoxy}]propionic acid; Pseudouridine TP 1-[3-{2-(2-[2-ethoxy]-ethoxy)-ethoxy}]propionic acid; Pseudouridine TP 1-[3-{2-(2-ethoxy)-ethoxy}]propionic acid; Pseudouridine TP 1-methylphosphonic acid; Pseudouridine TP 1-methylphosphonic acid diethyl ester; Pseudo-UTP-N1-3-propionic acid; Pseudo-UTP-N1-4-butanoic acid; Pseudo-UTP-N1-5-pentanoic acid; Pseudo-UTP-N1-6-hexanoic acid; Pseudo-UTP-N1-7-heptanoic acid; Pseudo-UTP-N1-methyl-p-benzoic acid; Pseudo-UTP-N1-p-benzoic acid; Wybutosine; Hydroxywybutosine; Isowyosine; Peroxywybutosine; undermodified hydroxywybutosine; 4-demethylwyosine; 2,6-(diamino)purine;1-(aza)-2-(thio)-3-(aza)-phenoxazin-1-yl: 1,3-(diaza)-2-(oxo)-phenthiazin-1-yl;1,3-(diaza)-2-(oxo)-phenoxazin-1-yl;1,3,5-(triaza)-2,6-(dioxa)-naphthalene; 2 (amino)purine;2,4,5-(trimethyl)phenyl;2′methyl, 2′amino, 2′azido, 2′fluro-cytidine;2′methyl, 2′amino, 2′azido, 2′fluro-adenine;2′methyl, 2′amino, 2′azido, 2′fluro-uridine;2′-amino-2′-deoxyribose; 2-amino-6-Chloro-purine; 2-aza-inosinyl; 2′-azido-2′-deoxyribose; 2′fluoro-2′-deoxyribose; 2′-fluoro-modified bases; 2′-O-methyl-ribose; 2-oxo-7-aminopyridopyrimidin-3-yl; 2-oxo-pyridopyrimidine-3-yl; 2-pyridinone; 3 nitropyrrole; 3-(methyl)-7-(propynyl)isocarbostyrilyl; 3-(methyl)isocarbostyrilyl; 4-(fluoro)-6-(methyl)benzimidazole; 4-(methyl)benzimidazole; 4-(methyl)indolyl; 4,6-(dimethyl)indolyl; 5 nitroindole; 5 substituted pyrimidines; 5-(methyl)isocarbostyrilyl; 5-nitroindole; 6-(aza)pyrimidine; 6-(azo)thymine; 6-(methyl)-7-(aza)indolyl; 6-chloro-purine; 6-phenyl-pyrrolo-pyrimidin-2-on-3-yl; 7-(aminoalkylhydroxy)-1-(aza)-2-(thio)-3-(aza)-phenthiazin-1-yl; 7-(aminoalkylhydroxy)-1-(aza)-2-(thio)-3-(aza)-phenoxazin-1-yl; 7-(aminoalkylhydroxy)-1,3-(diaza)-2-(oxo)-phenoxazin-1-yl; 7-(aminoalkylhydroxy)-1,3-(diaza)-2-(oxo)-phenthiazin-1-yl; 7-(aminoalkylhydroxy)-1,3-(diaza)-2-(oxo)-phenoxazin-1-yl; 7-(aza)indolyl; 7-(guanidiniumalkylhydroxy)-1-(aza)-2-(thio)-3-(aza)-phenoxazinl-yl; 7-(guanidiniumalkylhydroxy)-1-(aza)-2-(thio)-3-(aza)-phenthiazin-1-yl; 7-(guanidiniumalkylhydroxy)-1-(aza)-2-(thio)-3-(aza)-phenoxazin-1-yl; 7-(guanidiniumalkylhydroxy)-1,3-(diaza)-2-(oxo)-phenoxazin-1-yl; 7-(guanidiniumalkyl-hydroxy)-1,3-(diaza)-2-(oxo)-phenthiazin-1-yl; 7-(guanidiniumalkylhydroxy)-1,3-(diaza)-2-(oxo)-phenoxazin-1-yl; 7-(propynyl)isocarbostyrilyl; 7-(propynyl)isocarbostyrilyl, propynyl-7-(aza)indolyl; 7-deaza-inosinyl; 7-substituted 1-(aza)-2-(thio)-3-(aza)-phenoxazin-1-yl; 7-substituted 1,3-(diaza)-2-(oxo)-phenoxazin-1-yl; 9-(methyl)-imidizopyridinyl; Aminoindolyl; Anthracenyl; bis-ortho-(aminoalkylhydroxy)-6-phenyl-pyrrolo-pyrimidin-2-on-3-yl; bis-ortho-substituted-6-phenyl-pyrrolo-pyrimidin-2-on-3-yl; Difluorotolyl; Hypoxanthine; Imidizopyridinyl; Inosinyl; Isocarbostyrilyl; Isoguanisine; N2-substituted purines; N6-methyl-2-amino-purine; N6-substituted purines; N-alkylated derivative; Napthalenyl; Nitrobenzimidazolyl; Nitroimidazolyl; Nitroindazolyl; Nitropyrazolyl; Nubularine; 06-substituted purines; O-alkylated derivative; ortho-(aminoalkylhydroxy)-6-phenyl-pyrrolo-pyrimidin-2-on-3-yl; ortho-substituted-6-phenyl-pyrrolo-pyrimidin-2-on-3-yl; Oxoformycin TP; para-(aminoalkylhydroxy)-6-phenyl-pyrrolo-pyrimidin-2-on-3-yl; para-substituted-6-phenyl-pyrrolo-pyrimidin-2-on-3-yl; Pentacenyl; Phenanthracenyl; Phenyl; propynyl-7-(aza)indolyl; Pyrenyl; pyridopyrimidin-3-yl; pyridopyrimidin-3-yl, 2-oxo-7-amino-pyridopyrimidin-3-yl; pyrrolo-pyrimidin-2-on-3-yl; Pyrrolopyrimidinyl; Pyrrolopyrizinyl; Stilbenzyl; substituted 1,2,4-triazoles; Tetracenyl; Tubercidine; Xanthine; Xanthosine-5′-TP; 2-thio-zebularine; 5-aza-2-thio-zebularine; 7-deaza-2-amino-purine; pyridin-4-one ribonucleoside; 2-Amino-riboside-TP; Formycin A TP; Formycin B TP; Pyrrolosine TP; 2′-OH-ara-adenosine TP; 2′-OH-ara-cytidine TP; 2′-OH-ara-uridine TP; 2′-OH-ara-guanosine TP; 5-(2-carbomethoxyvinyl)uridine TP; and N6-(19-Amino-pentaoxanonadecyl)adenosine TP.

In some embodiments, the polynucleotide (e.g., RNA polynucleotide, such as mRNA polynucleotide) includes a combination of at least two (e.g., 2, 3, 4 or more) of the aforementioned modified nucleobases.

In some embodiments, the mRNA comprises at least one chemically modified nucleoside. In representative examples, the at least one chemically modified nucleoside is selected from the group consisting of pseudouridine (p), 2-thiouridine (s2U), 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methoxyuridine, 2′-O-methyl uridine, 1-methyl-pseudouridine (m1ψ), 1-ethyl-pseudouridine (e1ψ), 5-methoxy-uridine (mo5U), 5-methyl-cytidine (m5C), α-thio-guanosine, α-thio-adenosine, 5-cyano uridine, 4′-thio uridine 7-deaza-adenine, 1-methyl-adenosine (m1A), 2-methyl-adenine (m2A), N6-methyl-adenosine (m6A), and 2,6-Diaminopurine, (I), 1-methyl-inosine (m1I), wyosine (imG), methylwyosine (mimG), 7-deaza-guanosine, 7-cyano-7-deaza-guanosine (preQO), 7-aminomethyl-7-deaza-guanosine (preQ1), 7-methyl-guanosine (m7G), 1-methyl-guanosine (m1G), 8-oxo-guanosine, 7-methyl-8-oxo-guanosine, 2,8-dimethyladenosine, 2-geranylthiouridine, 2-lysidine, 2-selenouridine, 3-(3-amino-3-carboxypropyl)-5,6-dihydrouridine, 3-(3-amino-3-carboxypropyl)pseudouridine, 3-methylpseudouridine, 5-(carboxyhydroxymethyl)-2′-O-methyluridine methyl ester, 5-aminomethyl-2-geranylthiouridine, 5-aminomethyl-2-selenouridine, 5-aminomethyluridine, 5-carbamoylhydroxymethyluridine, 5-carbamoylmethyl-2-thiouridine, 5-carboxymethyl-2-thiouridine, 5-carboxymethylaminomethyl-2-geranylthiouridine, 5-carboxymethylaminomethyl-2-selenouridine, 5-cyanomethyluridine, 5-hydroxycytidine, 5-methylaminomethyl-2-geranylthiouridine, 7-aminocarboxypropyl-demethylwyosine, 7-aminocarboxypropylwyosine, 7-aminocarboxypropylwyosine methyl ester, 8-methyladenosine, N4,N4-dimethylcytidine, N6-formyladenosine, N6-hydroxymethyladenosine, agmatidine, cyclic N6-threonylcarbamoyladenosine, glutamyl-queuosine, methylated undermodified hydroxywybutosine, N4,N4,2′-O-trimethylcytidine, geranylated 5-methylaminomethyl-2-thiouridine, geranylated 5-carboxymethylaminomethyl-2-thiouridine, Qbase, preQObase, preQ1base, and two or more combinations thereof. In some embodiments, the at least one chemically modified nucleoside is selected from the group consisting of pseudouridine, 1-methyl-pseudouridine, 1-ethyl-pseudouridine, 5-methylcytosine, 5-methoxyuridine, and a combination thereof. In some embodiments, the polynucleotide (e.g., RNA polynucleotide, such as mRNA polynucleotide) includes a combination of at least two (e.g., 2, 3, 4 or more) of the aforementioned modified nucleobases.

In some embodiments, the RNA polynucleotide is formulated within a lipid nanoparticle. 5′-capping of polynucleotides may be completed concomitantly during the in vitro-transcription reaction using the following chemical RNA cap analogs to generate the 5′-guanosine cap structure according to manufacturer protocols: 3′-O-Me-m7G(5′)ppp(5′) G [the ARCA cap]; G(5′)ppp(5′)A; G(5′)ppp(5′)G; m7G(5′)ppp(5′)A; m7G(5′)ppp(5′)G (New England BioLabs, Ipswich, Mass.). 5′-capping of modified RNA may be completed post-transcriptionally using a Vaccinia Virus Capping Enzyme to generate the “Cap 0” structure: m7G(5′)ppp(5′)G (New England BioLabs, Ipswich, Mass.). Cap 1 structure may be generated using both Vaccinia Virus Capping Enzyme and a 2′-O methyl-transferase to generate: m7G(5′)ppp(5′)G-2′-O-methyl. Cap 2 structure may be generated from the Cap 1 structure followed by the 2′-O-methylation of the 5′-antepenultimate nucleotide using a 2′-O methyl-transferase. Cap 3 structure may be generated from the Cap 2 structure followed by the 2′-O-methylation of the 5′-preantepenultimate nucleotide using a 2′-O methyl-transferase. Enzymes may be derived from a recombinant source.

### 2.1.5 Nucleic Acid Constructs

The present disclosure also contemplates nucleic acid constructs for endogenous production of a polypeptide (e.g., 6PGD, HDCA7), wherein the nucleic acid constructs comprise an ORF encoding the polypeptide, which ORF is operably connected to a regulatory element that is suitably operable in a cell such as an APC or other immune cell. The nucleic acid constructs can be self-replicating extra-chromosomal vectors/replicons (e.g., plasmids) or vectors that integrate into a host genome. In specific embodiments, the nucleic acid constructs are viral vectors. Exemplary viral vectors include retroviral vectors, lentiviral vectors, poxvirus vectors, vaccinia virus vectors, adenovirus vectors, adenovirus-associated virus vectors, herpes virus vectors, flavivirus vectors, and alphavirus vectors. Viral vectors may be live, attenuated, replication conditional or replication deficient, and typically is a non-pathogenic (defective), replication competent viral vector.

### 2.2 Particles and Delivery Vehicles

In accordance with the present disclosure, the anti-inflammatory agents disclosed herein are useful in therapeutic agents for inhibiting or reducing pro-inflammatory activity of an immune cells such as an APC, or for treating, inhibiting or reducing, or slowing the progression of, inflammation in a subject. In some embodiments, the anti-inflammatory agents are, or are included as, cargos of particles or delivery vehicles.

Exemplary particles include particles that are capable of being taken up by immune cells such as APCs. Particles may be microparticles or nanoparticles, representative examples of which include liposomes, lipid-based particles, lipid carriers such as lipidoids, lipoplexes, polymeric particles, inorganic particles, inorganic particles coated with polymer or lipid, micelles, filomicelles, exosomes, peptide carriers, lipoproteins, lipid-coated bubbles, polymersomes, niosomes, nanotubes, carbon nanoassemblies, paramagnetic particles, ferromagnetic particles, microvesicles, dendrimers, hyperbranched polymers and conjugates.

In specific embodiments, the particles are nanoparticles. The nanoparticles may have any desired size for the intended use. The nanoparticles may have any diameter from 10 nm to 1,000 nm. The nanoparticle can have a diameter from 10 nm to 900 nm, from 10 nm to 800 nm, from 10 nm to 700 nm, from 10 nm to 600 nm, from 10 nm to 500 nm, from 20 nm from 500 nm, from 30 nm to 500 nm, from 40 nm to 500 nm, from 50 nm to 500 nm, from 50 nm to 400 nm, from 50 nm to 350 nm, from 50 nm to 300 nm, or from 50 nm to 200 nm. In preferred embodiments the nanoparticles can have a diameter less than 400 nm, less than 300 nm, or less than 200 nm. The preferred range is between 50 nm and 300 nm.

Nanoparticles can be polymeric particles, non-polymeric particles (e.g., a metal particle, quantum dot, ceramic, inorganic material, bone, etc.), liposomes, exosomes, micelles, polymeric micelles, viral particles, hybrids thereof, and/or combinations thereof. In some embodiments, the nanoparticles are, but not limited to, one or a plurality of lipid-based nanoparticles, polymeric nanoparticles, metallic nanoparticles, surfactant-based emulsions, dendrimers, buckyballs, nanowires, virus-like particles, peptide or protein-based particles (such as albumin nanoparticles) and/or nanoparticles that are developed using a combination of nanomaterials such as lipid-polymer nanoparticles. In some embodiments, nanoparticles can comprise one or more polymers or co-polymers.

Nanoparticles may be a variety of different shapes, including but not limited to spheroidal, cubic, pyramidal, oblong, cylindrical, toroidal, and the like. Nanoparticles can comprise one or more surfaces.

In some embodiments, the nanoparticles present within a population, e.g., in a composition, can have substantially the same shape and/or size (i.e., they are “monodisperse”). For example, the particles can have a distribution such that no more than about 5% or about 10% of the nanoparticles have a diameter greater than about 10% greater than the average diameter of the particles, and in some cases, such that no more than about 8%, about 5%, about 3%, about 1%, about 0.3%, about 0.1%, about 0.03%, or about 0.01% have a diameter greater than about 10% greater than the average diameter of the nanoparticles.

In some embodiments, the diameter of no more than 25% of the nanoparticles varies from the mean nanoparticle diameter by more than 150%, 100%, 75%, 50%, 25%, 20%, 10%, or 5% of the mean nanoparticle diameter. It is often desirable to produce a population of nanoparticles that is relatively uniform in terms of size, shape, and/or composition so that most of the nanoparticles have similar properties. In some embodiments, a population of nanoparticles can be heterogeneous with respect to size, shape, and/or composition. In this regard, see, e.g., WO 2007/150030, which is incorporated herein by reference in its entirety.

### 2.2.1 Lipid-Based Particles

In some embodiments, the particles (e.g., nanoparticles or microparticles) may comprise a lipid-based particle, or cationic lipid and optionally a biodegradable polymer. In some embodiments, a particle may comprise a liposome. In some embodiments, a particle may comprise a lipid bilayer. In some embodiments, a nanoparticle may comprise a lipid monolayer. In some embodiments, a particle may comprise a micelle. In these delivery vehicles, the anti-inflammatory agent disclosed herein is in the hollow core of the liposome or the micelle.

Methods of preparing liposomes, lipid discs, and other lipid nanoparticles and the like are generally known in the art, such as by methods described by Zhu et al. (US P20140348900), Cullis et al. (US 20140328759), See Wang et al., ACS Synthetic Biology, 1, 403-07 (2012); Wang et al., PNAS, 113(11) 2868-2873 (2016); Spuch and Navarro, Journal of Drug Delivery, vol. 2011, Article ID 469679, 12 pages, 2011. doi:10.1155/2011/469679; PCT Patent Publication WO 2008/042973; U.S. Pat. No. 8,071,082; and PCT Patent Publication WO 2014/186366 A1 (US Patent Publication No. US20160082126), the compositions and techniques of which can be used with and/or adapted for use with the present disclosure.

The cationic lipid may comprise 1,2-dioleoyl-3-trimethylammonium-propane (DOTAP). In some embodiments, the hydrophilic polymer comprises ethylene glycol or polyethylene glycol. In some embodiments, the particle further comprises a lipoprotein, preferably cholesterol.

In some embodiments, the anti-inflammatory agent(s) disclosed herein can be delivered by a particle, complex or other composition, such as any of those described, for example, by:
U.S. Pat. Appl. Pub. No. 20190091164, which describes nanoparticles comprising an endo-lysosomal escape agent, a nucleic acid, and a polymer;
U.S. Pat. No. 9,895,378, which discloses nanoparticles comprising a substantially hydrophobic acid and a therapeutic agent (1-(4-{[4-(dimethylamino)piperidin-1-yl]carbonyl}phenyl)-3-[4-(4,6-dimorpholin-4-yl-1,3,5-triazin-2-yl)phenyl]urea), or pharmaceutically acceptable salts thereof, and a polymer;
U.S. Pat. No. 9,579,386, which teaches nanoparticles having about 0.2 to about 35 weight percent of a therapeutic agent; and about 10 to about 99 weight percent of biocompatible polymer such as a diblock poly(lactic) acid-poly(ethylene)glycol;
U.S. Pat. No. 9,498,443, which describes compositions comprising a sugar, a cyclodextrin, and polymeric nanoparticles comprising a copolymer and a therapeutic agent which, upon reconstitution, have low levels of greater than 10 micron size particles;
U.S. Pat. Appl. Pub. No. 20150232883. In some embodiments, the particle or other composition described by U.S. Pat. Publ. No. 20150232883 can include a surfactant, a lipid, a protein, or a combination thereof. In some embodiments, the surfactant can be a cationic lipid;
PCT Patent Publication WO2015089419 A2 and documents cited therein, such as 7C1 (see, e.g., James E. Dahlman and Carmen Barnes et al., Nature Nanotechnology (2014) published online 11 May 2014, doi:10.1038/nnano.2014.84);
U.S. Pat. No. 9,301,923, which discloses cationic lipids and stealth lipids, and formulations containing them;
U.S. Pat. Appl. Pub. No. 20160174546, which describes nanolipid delivery particles and systems;
U.S. Pat. Appl. Pub. No. 20140301951, which describes at least a protocell nanostructure composed of a porous particle core comprising a plurality of pores and at least one lipid bilayer surrounding the porous particle core to form a protocell, wherein the protocell is capable of loading one or more cargo components to the plurality of pores of the porous particle core and releasing the one or more cargo components from the porous particle core across the surrounding lipid bilayer;
U.S. Pat. Appl. Pub. No. 20110293703, which describe lipidoid compounds, including aminoalcohol lipidoid compounds;
U.S. Pat. No. 5,985,309, which discloses particles incorporating a surfactant and/or a hydrophilic or hydrophobic complex of a positively or negatively charged therapeutic agent and a charged molecule of opposite charge;
PCT Patent Publication WO 2012/135025, which describes conjugated lipomers.
PCT Publications WO 2005/105152 (PCT/EP2005/004920), WO 2006/069782 (PCT/EP2005/014074), WO 2007/121947 (PCT/EP2007/003496), and WO 2015/082080 (PCT/EP2014/003274); and
U.S. Pat. Appl. Pub. Nos. 20140308304, 20150140070, 20160200779, 20150118216, 20150071903, and 20150071903, and PCT Patent Publication WO 2013/093648, which describes lipid-containing particles.

In some embodiments, the lipid particle can be a liposome such as a Trojan Horse liposome. See e.g., http://cshprotocols.cshlp.org/content/2010/4/pdb.prot5407.long,

In some embodiments, the lipid particle can be a stable nucleic-acid-lipid particle (SNALP). See, e.g., Morrissey et al., Nature Biotechnology, Vol. 23, No. 8, August 2005, the teachings of which can be applied and/or adapted to the present disclosure.

In some embodiments, the lipid particles as described in, e.g., Wang et al., J. Control Release, 2017 Jan. 31. pii: 50168-3659(17)30038-X. doi: 10.1016/j.jconre1.2017.01.037.; Altnoǧlu et al., Biomater Sci., 4(12):1773-80, Nov. 15, 2016; Wang et al., PNAS, 113(11):2868-73 Mar. 15, 2016; Wang et al., PloS One, 10(11): e0141860. doi: 10.1371/journal.pone.0141860. eCollection 2015, Nov. 3, 2015; Takeda et al., Neural Regen Res. 10(5):689-90, May 2015; Wang et al., Adv. Healthc Mater., 3(9):1398-403, September 2014; and Wang et al., Agnew Chem Int Ed Engl., 53(11):2893-8, Mar. 10, 2014, can be used/adapted to deliver one or more of the anti-inflammatory agent(s) described herein.

### 2.2.2 Polymer-Based Particles

The anti-inflammatory agent(s) described herein may be delivered using polymer-based particles (e.g., nanoparticles or microparticles). In some embodiments, the polymer-based particles may mimic a viral mechanism of membrane fusion. The polymer-based particles may be a synthetic copy of Influenza virus machinery and form transfection complexes with various types of nucleic acids (siRNA, miRNA, plasmid DNA or shRNA, mRNA) that cells take up via the endocytosis pathway, a process that involves the formation of an acidic compartment. The low pH in late endosomes acts as a chemical switch that renders the particle surface hydrophobic and facilitates membrane crossing. Once into the cytosol, the particle can release its payload for cellular action. This active endosome escape technology is safe and can maximize transfection efficiency, as it uses a natural uptake pathway. In some embodiments, the polymer-based particles may comprise alkylated and carboxyalkylated branched polyethylenimine. In some examples, the polymer-based particles are VIROMER, e.g., VIROMER RNAi, VIROMER RED, VIROMER mRNA, VIROMER CRISPR. Example methods of delivering the systems and compositions herein include those described in Bawage S S et al., Synthetic mRNA expressed Cas13a mitigates RNA virus infections, www.biorxiv.org/content/10.1101/370460v1.full doi: doi.org/10.1101/370460, Viromer® RED, a powerful tool for transfection of keratinocytes. doi: 10.13140/RG.2.2.16993.61281, Viromer® Transfection—Factbook 2018: technology, product overview, users' data, doi:10.13140/RG.2.2.23912.16642.

In some embodiments, the anti-inflammatory agent(s) disclosed herein can be delivered by a polymer-based particle or complex, such as any as set forth in:
U.S. Pat. Appl. Pub. No. 20230165971, which discloses silica organic nanoparticles, which typically have a hollow core and a surface morphology with projections forming a plurality of spikes or finger-like structures on the surface between which matter can become enmeshed;
U.S. Pat. Appl. Pub. No. 20200392005, which describes processes for making hollow inorganic nanoparticles typically having a rough or “spiky” surface morphology;
U.S. Pat. Publ. No. 20110212179, which at least provides bimodal porous polymer microspheres.
U.S. Pat. Appl. Pub. No. 20130302401, which describes at least a class of poly(beta-amino alcohols.
U.S. Pat. No. 8,709,843, which describes at least targeted lipo-polymeric particles.
U.S. Pat. No. 6,007,845, which describes at least particles composed of multiblock copolymers.
U.S. Pat. No. 5,543,158, which describes at least biodegradable injectable particles having a biodegradable solid core containing a biologically active material and poly(alkylene glycol) moieties on the surface.
U.S. Pat. Appl. Pub. Nos. 20130252281, US 20130245107, and US 20130244279, which describe at least PLGA microparticles.

### 2.2.3 Dendrimers

In some embodiments, the anti-inflammatory agent(s) disclosed herein can be delivered via a dendrimer nanoparticle or a modified dendrimer nanoparticle, such as those described in US Patent Publication Nos. US 20170079916, US 20050019923, and US 20080267903. Dendrimers are synthetic 3-dimensional macromolecules that are prepared in a step-wise fashion from simple branched monomer units, the nature and functionality of which can be easily controlled and varied. Dendrimers are synthesized from the repeated addition of building blocks to a multifunctional core (divergent approach to synthesis), or towards a multifunctional core (convergent approach to synthesis) and each addition of a 3-dimensional shell of building blocks leads to the formation of a higher generation of the dendrimers.

### 2.2.4 Sugar-Based Particles

In some embodiments, the particle for delivery of the anti-inflammatory agent(s) disclosed herein can be a sugar-based particle, an illustrative example of which includes GalNAc, as disclosed, for example, in PCT Publication WO 2014/118272 and Nair, J K et al. (2014, Journal of the American Chemical Society 136 (49), 16958-16961).

The disclosed anti-inflammatory agent(s) may be delivered by any lipid-protein-sugar particle, such as by any of those described in US Patent Publication No. US 20020150626. In some embodiments, the lipid-protein-sugar particle can encapsulate the one or more RNA molecules. Encapsulation can be accomplished by contacting the polynucleotide with a lipid, a protein, and a sugar; and spray drying mixture of the polynucleotide, the lipid, the protein, and the sugar to make microparticles.

### 2.2.5 Spherical Nucleic Acid (SNA)

In some embodiments, the particle for delivery of the anti-inflammatory agent(s) disclosed herein can be a SNA. SNAs are three dimensional nanostructures that can be composed of densely functionalized and highly oriented nucleic acids that can be covalently attached to the surface of spherical nanoparticle cores. The core of the spherical nucleic acid can impart the conjugate with specific chemical and physical properties, and it can act as a scaffold for assembling and orienting the oligonucleotides into a dense spherical arrangement that gives rise to many of their functional properties, distinguishing them from all other forms of matter.

In some embodiments, the SNA can be any of those set forth in Cutler et al., J. Am. Chem. Soc. 2011 133:9254-9257, Hao et al., Small. 2011 7:3158-3162, Zhang et al., ACS Nano. 2011 5:6962-6970, Cutler et al., J. Am. Chem. Soc. 2012 134:1376-1391, Young et al., Nano Lett. 2012 12:3867-71, Zheng et al., Proc. Natl. Acad. Sci. USA. 2012 109:11975-80, Mirkin, Nanomedicine 2012 7:635-638 Zhang et al., J. Am. Chem. Soc. 2012 134:16488-1691, Weintraub, Nature 2013 495:S14-S16, Choi et al., Proc. Natl. Acad. Sci. USA. 2013 110(19):7625-7630, Jensen et al., Sci. Transl. Med. 5, 209ra152 (2013) and Mirkin, et al., Small, 10:186-192, which can be applied and/or adapted for generation of and delivery of one or more systems or components thereof, complexes, polypeptides, polynucleotides, vectors, virus particles, and combinations thereof described herein.

### 2.2.6 Self-Assembling Nanoparticles

In some embodiments, the particle for delivery of the anti-inflammatory agent(s) disclosed herein can be a self-assembling nanoparticle or nanoplex. Representative examples of self-assembling nanoparticles or nanoplexes are described in Bartlett et al. PNAS, Sep. 25, 2007, vol. 104, no. 39; Schiffelers et al., Nucleic Acids Research, 2004, Vol. 32, No. 19; and Nature, Vol 464, 15 Apr. 2010.

### 2.2.7 Nanoclews

In some embodiments, the particle for delivery of the disclosed anti-inflammatory agent(s) is a nanoclew, non-limiting examples of which are described by Sun W et al., Cocoon-like self-degradable DNA nanoclew for anticancer drug delivery, J Am Chem Soc. 2014 Oct. 22; 136(42):14722-5. doi: 10.1021/ja5088024. Epub 2014 Oct. 13.; or in Sun W et al., Self-Assembled DNA Nanoclews for the Efficient Delivery of CRISPR-Cas9 for Genome Editing., Angew Chem Int Ed Engl. 2015 Oct. 5; 54(41):12029-33. doi: 10.1002/anie.201506030. Epub 2015 Aug. 27.

### 2.2.8 Hybrid Particles

In some embodiments, the delivery vehicle is a particle (e.g., a nanoparticle or microparticle) comprising a water-insoluble polymeric core.

The water-insoluble polymeric core can comprise a variety of materials. The water-insoluble polymer can comprise homopolymers (i.e., synthesized from hydrophobic monomers (e.g., styrene, methyl methacrylate, glycidyl methacrylate, DL-lactide, and the like)), random copolymers (i.e., synthesized from two or more monomers (e.g., styrene, methyl methacrylate, glycidyl methacrylate, DL-lactide, acrylic acid, methacrylic acid, 2-hydroxyethyl acrylate, and the like)), block polymers (i.e., synthesized from two or more monomers (e.g., styrene, methyl methacrylate, glycidyl methacrylate, DL-lactide, acrylic acid, methacrylic acid, 2-hydroxyethyl acrylate, and the like)), graft polymers (e.g., synthesized from artificial polymers (polyacrylic acid, polyglycidyl methacrylate, and the like) and/or natural polymers (e.g., dextran, starch, chitosan, and the like) with functional pendent groups (e.g., amino, carboxylate, hydroxyl, epoxy groups, and the like)), and/or branched polymers (e.g., a hyperbranched polyester with multifunctional alcohol building block and 2,2-bis(methylol)propionic acid branching units, such as Boltorn™ H40).

Non-limiting exemplary polymers that can be included in the polymeric core include polymer systems that are approved for use in humans, e.g., poly(glycolic acid), poly(lactic acid), poly(caprolactone), poly(lactide-co-glycolide), poly(ortho ester) II, poly(alkyl cyanoacrylate), desaminotyrosyl octyl ester, polyphosphoesters, polyester amides, polyurethanes, and lipids. Other non-limiting examples of polymers that the core can comprise include: chitosan; acrylates copolymer; acrylic acid-isooctyl acrylate copolymer; ammonio methacrylate copolymer; ammonio methacrylate copolymer type A; ammonio methacrylate copolymer type B; butyl ester of vinyl methyl ether/maleic anhydride copolymer (125,000 molecular weight); carbomer homopolymer type A (allyl pentaerythritol crosslinked); carbomer homopolymer type B (allyl sucrose crosslinked); cellulosic polymers; dimethylaminoethyl methacrylate-butyl methacrylate-methyl methacrylate copolymer; dimethylsiloxane/methylvinylsiloxane copolymer; divinylbenzene styrene copolymer; ethyl acrylate-methacrylic acid copolymer; ethyl acrylate and methyl methacrylate copolymer (2:1; 750,000 molecular weight); ethylene vinyl acetate copolymer; ethylene-propylene copolymer; ethylene-vinyl acetate copolymer (28% vinyl acetate); glycerin polymer solution i-137; glycerin polymer solution im-137; hydrogel polymer; ink/polyethylene terephthalate/aluminum/polyethylene/sodium polymethacrylate/ethylene vinyl acetate copolymer; isooctyl acrylate/acrylamide/vinyl acetate copolymer; Kollidon® VA 64 polymer; methacrylic acid-ethyl acrylate copolymer (1:1) type A; methacrylic acid-methyl methacrylate copolymer (1:1); methacrylic acid-methyl methacrylate copolymer (1:2); methacrylic acid copolymer; methacrylic acid copolymer type A; methacrylic acid copolymer type B; methacrylic acid copolymer type C; octadecene-1/maleic acid copolymer; PEG-22 methyl ether/dodecyl glycol copolymer; PEG-45/dodecyl glycol copolymer; Polyester polyamine copolymer; poly(ethylene glycol) 1,000; poly(ethylene glycol) 1,450; poly(ethylene glycol) 1,500; poly(ethylene glycol) 1,540; poly(ethylene glycol) 200; poly(ethylene glycol) 20,000; poly(ethylene glycol) 200,000; poly(ethylene glycol) 2,000,000; poly(ethylene glycol) 300; poly(ethylene glycol) 300-1,600; poly(ethylene glycol) 300-1,600; poly(ethylene glycol) 3,350; poly(ethylene glycol) 3,500; poly(ethylene glycol) 400; poly(ethylene glycol) 4,000; poly(ethylene glycol) 4,500; poly(ethylene glycol) 540; poly(ethylene glycol) 600; poly(ethylene glycol) 6,000; poly(ethylene glycol) 7,000; poly(ethylene glycol) 7,000,000; poly(ethylene glycol) 800; poly(ethylene glycol) 8,000; poly(ethylene glycol) 900; polyvinyl chloride-polyvinyl acetate copolymer; povidone acrylate copolymer; povidone/eicosene copolymer; polyoxy(methyl-1,2-ethanediyl), alpha-hydro-omega-hydroxy-, polymer with 1,1′-methylenebis[4-isocyanatocyclohexane]copolymer; polyvinyl methyl ether/maleic acid copolymer; styrene/isoprene/styrene block copolymer; vinyl acetate-crotonic acid copolymer; {poly[(9,9-di-n-octylfluorenyl-2,7-diyl)-alt-(benzo[2,1,3]thiadiazol-4,8-diyl)]}, and {poly[2,6-(4,4-bis-(2-ethylhexyl)-4H-cyclopenta [2,1-b;3,4-b′]dithiophene)-alt-4,7(2,1,3-benzothiadiazole)]}.

In some embodiments, the water-insoluble core comprises a hydrophobic polymer. Non-limiting examples of hydrophobic polymers include, but are not limited to: polylactic acid (PLA), polypropylene oxide, poly(lactide-co-glycolide) (PLGA), poly(epsilon-caprolactone), poly(ethylethylene), polybutadiene, polyglycolide, polymethylacrylate, polyvinylbutylether, polystyrene, polycyclopentadienyl-methylnorbornene, polyethylenepropylene, polyethylethylene, polyisobutylene, polysiloxane, a polymer of any of the following: methyl acrylate, ethyl acrylate, propyl acrylate, n-butyl acrylate, isobutyl acrylate, 2-ethyl acrylate, t-butyl acrylate, methacrylates (e.g., ethyl methacrylate, n-butyl methacrylate, and isobutyl methacrylate), acrylonitriles, methacrylonitrile, vinyls (e.g., vinyl acetate, vinylversatate, vinylpropionate, vinylformamide, vinylacetamide, vinylpyridines, and vinyllimidazole), aminoalkyls (e.g., aminoalkylacrylates, aminoalkylsmethacrylates, aminoalkyl(meth)acrylamides), styrenes, and lactic acids.

In some embodiments, the water-insoluble core comprises an amphipathic polymer. Amphipathic polymers contain a molecular structure containing one or more repeating units (monomers) connected by covalent bonds and the overall structure includes both hydrophilic (polar) and lipophilic (apolar) properties, e.g., at opposite ends of the molecule. In some embodiments, the amphipathic polymers are copolymers containing a first hydrophilic polymer and a first hydrophobic polymer. Several methods are known in the art for identifying an amphipathic polymer. For example, an amphipathic polymer (e.g., an amphipathic copolymer) can be identified by its ability to form micelles in an aqueous solvent and/or Langmuir Blodgett films.

In some embodiments, the amphipathic polymer (e.g., an amphipathic copolymer) contains a polymer selected from the group of: polyethylene glycol (PEG), polyethylene oxide, polyethyleneimine, diethyleneglycol, triethyleneglycol, polyalkylene glycol, polyalkyline oxide, polyvinyl alcohol, polyvinylpyrrolidone, polyvinylmethylether, polymethyloxazoline, polyethyloxazoline, polyhydroxypropyl-oxazoline, polyhydroxypropylmethacrylamide, polymethacrylamide, polydimethylacryl-amide, polyhydroxypropylmethacrylate, polyhydroxyethylacrylate, hydroxymethylcellulose, hydroxyethylcellulose, polyglycerine, polyaspartamide, polyoxyethlene-polyoxypropylene copolymer (poloxamer), a polymer of any of lecithin or carboxylic acids (e.g., acrylic acid, methacrylic acid, itaconic acid, and maleic acid), polyoxyethylenes, polyethyleneoxide, and unsaturated ethylenic monocarboxylic acids. In some embodiments, the amphipathic polymer contains a polymer selected from the group of: polylactic acid (PLA), polypropylene oxide, poly(lactide-co-glycolide) (PLGA), poly(epsilon-caprolactone), poly(ethylethylene), polybutadiene, polyglycolide, polymethylacrylate, polyvinylbutylether, polystyrene, polycyclopentadienylmethylnorbornene, polyethylenepropylene, polyethylethylene, polyisobutylene, polysiloxane, and a polymer of any of the following: methyl acrylate, ethyl acrylate, propyl acrylate, n-butyl acrylate, isobutyl acrylate, 2-ethyl acrylate, t-butyl acrylate, methacrylates (e.g., ethyl methacrylate, n-butyl methacrylate, and isobutyl methacrylate), acrylonitriles, methacrylonitrile, vinyls (e.g., vinyl acetate, vinylversatate, vinylpropionate, vinylformamide, vinylacetamide, vinylpyridines, and vinyllimidazole), aminoalkyls (e.g., aminoalkylacrylates, aminoalkylsmethacrylates, and aminoalkyl(meth)acrylamides), styrenes, and lactic acids.

In some embodiments, the amphipathic polymer contains poly(ethylene glycol)-co-poly(D,L-lactic acid) (PLA-PEG), poly(ethylene glycol)-co-(poly(lactide-co-glycolide)) (PLGA-PEG) (e.g., the amphipathic polymer is PLGA-PEG), polystyrene-b-polyethylene oxide, polybutylacrylate-b-polyacrylic acid, or polybutylmethacrylate-b-polyethyleneoxide. Additional examples of amphipathic copolymers are described in U.S. Patent Application Publication No. 2004/0091546 (incorporated herein by reference in its entirety). Additional examples of amphipathic polymers (e.g., amphipathic copolymers) are known in the art.

In some embodiments, the water-insoluble core comprises a polymer comprising an aliphatic polyester polymer, e.g., polycaprolactone (PCL), polybutylene succinate (PBS), or a polyhydroxylalkanoate (PHA), such as polyhydroxybutyrate. Other examples include polylactic acid (PLA) and polyglycolic acid (PGA). In some embodiments, the aliphatic polyester polymer is selected from polylactic acids, polyglycolic acids, and copolymers of lactic acid and glycolic acid (PLGA). A copolymer of lactic acid and glycolic acid can comprise a range of ratios of lactic acid to glycolic acid monomers, for example, from about 1:9 to about 9:1, from about 1:4 to about 4:1, from about 3:7 to about 7:3, or from about 3:2 to about 2:3. In some embodiments, the ratio of lactic acid to glycolic acid monomers can be about 1:9; about 1:8; about 1:7; about 1:6; about 1:5; about 1:4; about 3:7; about 2:3; about 1:1; about 3:2; about 7:3; about 4:1; about 5:1; about 6:1; about 7:1; about 8:1; or about 9:1.

In some embodiments, the water-insoluble core comprises a fluorescent polymer. The fluorescent polymer can be one or more polymers selected from polyphenylenevinylenes (e.g., poly[(2-methoxy-5-(2-ethylhexyloxy)-1,4-phenylene-vinylene)-co-(4,4′-biphenylene-vinylene)]), polyfluorenes (e.g., poly(fluorene-co-phenylene) (PFP), poly(9,9-dioctylfluorenyl-2,7-diyl); copolymers such as poly[{9,9-dioctyl-2,7-divinylene-fluorenylene}-alt-co-{2-methoxy-5-(2-ethylhexyloxy)-1,4-phenylene}]), polythiophenes (e.g., poly(3-butylthiophene-2,5-diyl), poly(3-decyl-thiophene-2,5-diyl), poly[3-(2-ethyl-isocyanato-octadecanyl)thiophene], poly(3,3′″-didodecyl quarter thiophene), copolymers such as poly[(9,9-dihexylfluorenyl-2,7-diyl)-alt-co-(bithiophene)] and poly[(9,9-dioctylfluorenyl-2,7-diyl)-alt-co-(bithiophene)]), poly(p-phenyleneethylene)s (PPE), polydiacetylenes (PDA), and their derivatives. Additional non-limiting examples of fluorescent polymers include F8BT {poly[(9,9-di-n-octylfluorenyl-2,7-diyl)-alt-(benzo[2,1,3]thiadiazol-4,8-diyl)]} and PCPDTBT {poly[2,6-(4,4-bis-(2-ethylhexyl)-4H-cyclopenta [2,1-b;3,4-b′]dithiophene)-alt-4,7(2,1,3-benzothiadiazole)]}.

In some embodiments, the particles of the present disclosure can be prepared according to the methods similar to those described in WO 2018/089688, US20170362388, and US20170304213.

### 2.3 Targeting Moieties

The therapeutic agent may comprise a targeting moiety specific for an immune cells such as an APC. The targeting moiety includes and encompasses any molecule or moiety that is specific for a protein, sugar, or other molecule present on the surface of an immune cell (e.g., an APC). The targeting moiety is suitably selected from antigen-binding molecules, illustrative examples of which include antibodies and non-antibody targeting molecules.

In some embodiments, the targeted immune cell is an APC.

The targeted APC may be a macrophage, exemplary receptors and cell surface molecules of which include TLR4, CD14, CD35, CD11b/CD18, CD11a/CD18, CD64, CD32, CD16, mannose receptor (CD206) and macrophage scavenger receptor.

In other embodiments, the targeted APC is a dendritic cell. Non-limiting examples of dendritic cell receptors and cell surface molecules include dendritic cell-specific intercellular adhesion molecule-3-capture non-integrin (DC-SIGN), also known as CD209 or Clec4L, Clec9A, and mannose receptor (CD206).

In other embodiments, the targeted APC is a B-cell. Illustrative dendritic cell receptors and cell surface molecules include B-cell antigen receptor, CD20, CD19 and CD79b.

In some embodiments, the targeted immune cell is a T cell. Non-limiting examples of receptors and cell surface molecules of T cells include CD3, CD4, CD8, and TCR α and β chains.

The targeting moiety is suitably an antigen-binding molecule such as, but not limited to, an antibody, antigen-binding antibody fragment, or a non-antibody targeting molecule that binds specifically to a receptor or other molecule on the surface of an immune cells such as an APC. Antibodies contemplated by the present disclosure include whole antibodies and antigen-binding antibody fragments. Thus, antibodies may be selected from naturally occurring antibodies that comprise at least two heavy (H) chains and two light (L) chains inter-connected by disulfide bonds. Each heavy chain is comprised of a heavy chain variable region (abbreviated herein as V_{H}) and a heavy chain constant region. The heavy chain constant region is comprised of three domains, C_{H1}, C_{H2} and C_{H3}. Each light chain is comprised of a light chain variable region (abbreviated herein as VL) and a light chain constant region. The light chain constant region is comprised of one domain, CL. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen or epitope thereof. The constant regions of the antibodies may mediate the binding of the immunoglobulin to host tissues or factors, including various cells of the immune system (e.g., effector cells) and the first component (Clq) of the classical complement system. Non-limiting examples of antibodies include monoclonal antibodies, human antibodies, humanized antibodies, camelized antibodies, chimeric antibodies, bi-specific or multiple-specific antibody and anti-idiotypic (anti-Id) antibodies. The antibodies can be of any isotype (e.g., IgG, IgE, IgM, IgD, IgA and IgY), class (e.g., IgG1, IgG2, IgG3, IgG4, IgA1 and IgA2) or subclass.

Generally, antibody fragments include portions of an antibody including, for example, single-chain Fv (scFv), Fab fragments, monovalent fragments consisting of the V_{L}, V_{H}, C_{L} and C_{H1} domains; a F(ab)₂ fragment, bivalent fragments comprising two Fab fragments linked together by a disulfide bridge at the hinge region; Fd fragments consisting of the VH and CH1 domains; a Fv fragment consisting of the V_{L} and V_{H} domains of a single arm of an antibody; dAb fragments (Ward et al., 1989. Nature 341:544-546), which consists of a V_{H} domain; and an isolated complementarity determining region (CDR). Antibody fragments can also be incorporated into single domain antibodies, maxibodies, minibodies, intrabodies, diabodies, triabodies, tetrabodies, v-NAR and bis-scFv (see, e.g., Hollinger and Hudson, (2005) Nature Biotechnology 23: 1126-1136), and nanobodies. Antibody fragments can be incorporated into single chain molecules comprising a pair of tandem Fv segments (V_{H}-C_{H1}-VH-C_{H1}) which, together with complementary light chain polypeptides, form a pair of antigen binding regions (as disclosed, e.g., Zapata et al. (1995. Protein Eng. 8:1057-1062); and U.S. Pat. No. 5,641,870). In certain embodiments, a targeting moiety may be an affibody, avimer, aptamer or fynomer.

### 3. Pharmaceutical Compositions

Also provided herein are pharmaceutical compositions or formulations comprising a therapeutic agent disclosed herein, optionally an HDAC7-producing agent disclosed herein and a pharmaceutically acceptable carrier. These compositions can be prepared by mixing the active ingredients (e.g., therapeutic agent and/or HDAC7-producing agent) having the desired degree of purity with one or more optional pharmaceutically acceptable carriers (Remington's Pharmaceutical Sciences 16th edition, Osol, A. Ed. (1980)). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

The compositions disclosed herein may also contain further active ingredients as necessary for the particular indication being treated, preferably those with complementary activities that do not adversely affect each other. Such active ingredients are suitably present in combination in amounts that are effective for the purpose intended.

Depending on the specific conditions being treated, the formulations may be administered systemically or locally. Suitable routes may, for example, include oral, rectal, transmucosal, or intestinal administration; parenteral delivery, including intramuscular, subcutaneous, intramedullary injections, as well as intrathecal, direct intraventricular, intravenous, intraperitoneal, intranasal, or intraocular injections. Techniques for formulation and administration may be found in “Remington's Pharmaceutical Sciences”, Mack Publishing Co., Easton, Pa., latest edition.

The pharmaceutical forms suitable for injectable use include sterile injectable solutions or dispersions and sterile powders for the preparation of sterile injectable solutions. Such forms should be stable under the conditions of manufacture and storage and may be preserved against reduction, oxidation and microbial contamination.

### 4. Ancillary Active Agents

The pharmaceutical compositions of the present disclosure may further comprise at least one additional or ancillary active agent for treating inflammation. For example, the ancillary active agent may be an auxiliary anti-inflammatory agent, such as a steroidal anti-inflammatory agent or a non-steroidal anti-inflammatory agent.

In still other embodiments, the ancillary active agent is an analgesic agent, such as a non-opioid analgesic or an opioid analgesic. In representative examples of this type, the analgesic agent relieves pain by elevating the pain threshold without disturbing consciousness or altering other sensory modalities.

In some embodiments, the ancillary active agent is an antimicrobial agent, which includes without limitation compounds that kill or inhibit the growth of microorganisms such as viruses, bacteria, yeast, fungi, protozoa, etc. and thus include antibiotics, antifungals, antiprotozoals, antimalarials, antituberculotics and antivirals.

Alternatively, or in addition, one can administer compounds which inhibit the cytokine release syndrome or cytokine storm, anti-coagulants and/or platelet aggregation inhibitors that address blood clots, compounds which chelate iron ions released from hemoglobin by viruses such as COVID-19, cytochrome P-450 (CYP450) inhibitors and/or NOX inhibitors, as ancillary active agents.

Alternatively, or in addition, HMGB1 antibodies and/or COX-2 inhibitors can be used, which downregulate the cytokine storm. Examples of such compounds include Actemra (Roche). Celebrex (celecoxib), a COX-2 inhibitor, can be used. IL-8 (CXCL8) inhibitors can also be used. Chemokine receptor CCR2 antagonists can reduce pulmonary immune pathology.

Accordingly, the present disclosure encompasses co-administration of a therapeutic agent disclosed herein in concert with an ancillary active agent or intervention, as described for example above and elsewhere herein. It will be understood that, in embodiments comprising administration of the therapeutic agent with other agents, the dosages of the actives in the combination may on their own comprise an effective amount and the additional agent(s) may further augment the therapeutic benefit to the patient. Alternatively, the therapeutic agent and the additional agent(s) may together comprise an effective amount for treating an inflammation. It will also be understood that effective amounts may be defined in the context of particular treatment regimens, including, e.g., timing and number of administrations, modes of administrations, formulations, etc. In some embodiments, the therapeutic agent and optionally the ancillary active agent are administered on a routine schedule. Alternatively, the ancillary active agent may be administered as symptoms arise. A “routine schedule” as used herein, refers to a predetermined designated period of time. The routine schedule may encompass periods of time which are identical, or which differ in length, as long as the schedule is predetermined. For instance, the routine schedule may involve administration of the therapeutic agent on a daily basis, every two days, every three days, every four days, every five days, every six days, a weekly basis, a monthly basis or any set number of days or weeks there-between, every two months, three months, four months, five months, six months, seven months, eight months, nine months, ten months, eleven months, twelve months, etc. Alternatively, the predetermined routine schedule may involve concurrent administration of the therapeutic agent and the ancillary active agent on a daily basis for the first week, followed by a monthly basis for several months, and then every three months after that. Any particular combination would be covered by the routine schedule as long as it is determined ahead of time that the appropriate schedule involves administration on a certain day.

Dosing is dependent on severity and responsiveness of the inflammatory disease state to be treated, with the course of treatment lasting from several days to several months, or until a cure is achieved or a diminution of the disease state is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual therapeutic agent and can generally be estimated based on EC₅₀ₛ found to be effective in in vitro and in vivo animal models. In general, dosage is from 0.01 μg to 100 g per kg of body weight, and may be given once or more daily, weekly, monthly or yearly. Persons of ordinary skill in the art can easily estimate repetition rates for dosing based on measured residence times and concentrations of the drug in bodily fluids or tissues.

Toxicity and therapeutic efficacy of therapeutic agents can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit large therapeutic indices are preferred. The data obtained from these cell culture assays, and animal studies can be used in formulating a range of dosage for use in human. The dosage of such compounds lies preferably within a range of circulating concentrations that include the ED50 with little or no toxicity. The dosage may vary within this range depending upon the dosage form employed and the route of administration utilized. The exact formulation, route of administration and dosage can be chosen by the individual physician in view of the patient's condition. (See for example Fingl et al., 1975, in “The Pharmacological Basis of Therapeutics”, Ch. 1 p1).

Pharmaceutical compositions of the present disclosure may be provided in a kit. The kit may comprise additional components to assist in performing the methods of the present disclosure such as, for example, administration device(s), buffer(s), and/or diluent(s). The kits may include containers for housing the various components and instructions for using the kit components in the methods of the present disclosure.

### 5. Therapeutic Uses

The present disclosure also extends to the use of the disclosed herein therapeutic agents, optionally in combination with an HDAC7-producing agent and/or at least one ancillary active agent, for treating a subject with inflammation. The inflammation may be associated with an acute inflammatory condition or a chronic inflammatory condition.

Acute inflammatory conditions may be selected from cytokine release syndrome (CRS) or a cytokine storm, multisystem inflammatory syndrome in children (MIS-C), systemic inflammatory response syndrome (SIRS), acute respiratory distress syndrome (ARDS), severe acute respiratory syndrome (SARS), ischemia, ischemia-reperfusion injuries, hemorrhagic shock, transfusion related acute lung injury (TRALI), pancreatitis, dermatitis, gingivitis, acute meningitis, acute gastritis, acute sarcoidosis, pneumonia, acute allergic rhinitis, glomerulonephritis, acute liver injury, renal failure, acute kidney disease, acute bacterial, viral or fungal infection, acute graft versus host disease, acute gout, acute thyroiditis, nephritis, polyarteritis nodosa, necrotizing enterocolitis, endotoxemia, septicemia, toxic shock syndrome, and acute tissue injury.

In some embodiments, the acute inflammatory condition is associated with, or results from, trauma, burns, massive transfusion, radiation injury, reperfusion injury, traumatic nerve injury, spinal cord injury, aging, chemical exposure, oxidative damage to tissues or cardiopulmonary bypass.

Alternatively, the acute inflammatory condition may be associated with a cardiovascular disease. In illustrative embodiments of this type, the cardiovascular disease is selected from myocardial infarction, stroke, vasculitis, microvasculopathy, stable angina pectoris (SAP), unstable angina pectoris (UAP), acute arrhythmia, acute rheumatic heart disease, acute myocarditis, acute pericarditis, acute heart failure (aHF), acute ischemic heart disease (aIHD), sudden cardiac death (SCD), cardiac tamponade, cardiogenic shock, acute coronary artery syndrome and hypertensive crisis.

In other embodiments, the subject has or is at risk of developing a chronic inflammatory condition, representative examples of which include chronic liver disease, inflammatory bowel disease (IBD) (e.g., Crohn's disease or ulcerative colitis), asthma, byssinosis, asbestosis, pneumonitis, pneumonia, chronic meningitis, diabetes, chronic allergic rhinitis, chronic sarcoidosis, chronic kidney disease, chronic thyroiditis, sciatica, pulmonary fibrosis, cystic fibrosis, polymyositis, gout, and chronic obstructive pulmonary disorder (COPD).

In some embodiments, the chronic inflammatory condition is associated with chronic granulomatous disease (CGD).

Alternatively, the chronic inflammatory condition may be associated with an autoimmune disease, a neurodegenerative disease, or a cardiovascular disease. In particular embodiments, the autoimmune disease is selected from alopecia areata, ankylosing spondylitis, antiphospholipid syndrome, autoimmune Addison's disease, autoimmune diseases of the adrenal gland, autoimmune hemolytic anemia, autoimmune hepatitis, autoimmune oophoritis and orchitis, autoimmune thrombocytopenia, Behcet's disease, bullous pemphigoid, cardiomyopathy, celiac sprue-dermatitis, chronic fatigue immune dysfunction syndrome (CFIDS), chronic inflammatory demyelinating polyneuropathy, Churg-Strauss syndrome, cicatrical pemphigoid, CREST syndrome, cold agglutinin disease, discoid lupus, essential mixed cryoglobulinemia, fibromyalgia-fibromyositis, glomerulonephritis, Graves' disease, Guillain-Barre, Hashimoto's thyroiditis, pulmonary fibrosis, idiopathic thrombocytopenia purpura (ITP), IgA neuropathy, juvenile arthritis, lichen planus, lupus erthematosus, Meniere's disease, mixed connective tissue disease, multiple sclerosis, type 1 or immune-mediated diabetes mellitus, myasthenia gravis, pemphigus vulgaris, pernicious anemia, polyarteritis nodosa, polychrondritis, polyglandular syndromes, polymyalgia rheumatica, polymyositis and dermatomyositis, primary agammaglobulinemia, primary biliary cirrhosis, psoriasis, psoriatic arthritis, Raynauld's phenomenon, Reiter's syndrome, Rheumatoid arthritis, sarcoidosis, scleroderma, Sjogren's syndrome, stiff-man syndrome, systemic lupus erythematosus, lupus erythematosus, takayasu arteritis, temporal arteristis/giant cell arteritis, ulcerative colitis, uveitis, vasculitides such as dermatitis herpetiformis vasculitis, vitiligo, thyroid-associated ophthalmopathy (TAO) such as Grave's disease, Wegener's granulomatosis, and the like.

The neurodegenerative disease associated with chronic inflammation may be selected from Alzheimer's disease, amyotrophic lateral sclerosis (ALS), multiple sclerosis (MS), Friedreich ataxia, Huntington's disease, Lewy body disease, Parkinson's disease, Guillain-Barre syndrome, spinal muscular atrophy, Bell's Palsy, spinal cord injury, cerebral aneurysm, epilepsy, and seizures.

In some embodiments, the cardiovascular disease associated with chronic inflammation is selected from atherosclerosis, coronary artery disease (CAD), chronic rheumatic heart disease, peripheral artery disease, peripheral vascular disease (PAD), congenital heart disease (CHD), sickle cell anemia, congestive heart failure, myocardial ischemia, chronic arrhythmia, cardiomyopathy and thrombosis.

### 6. Representative Embodiments

1. A method for inhibiting or reducing pro-inflammatory activity of an antigen-presenting cell, the method comprising contacting the antigen-presenting cell with a therapeutic agent, the therapeutic agent comprising D-ribulose-5-phosphate (RL5P), a RL5P analog, a RL5P analog-producing agent and/or a RL5P-producing agent.
2. The method of embodiment 1, wherein the RL5P-producing agent is 6-phosphogluconate dehydrogenase (6PGD) and/or a nucleic acid molecule from which 6PGD is producible.
3. The method of embodiment 1 or embodiment 2, wherein the antigen-presenting cell is selected from a macrophage, a dendritic cell and a B-cell.
4. The method of embodiment 3, wherein the antigen-presenting cell is a macrophage.
5. The method of any one of embodiments 1 to 4, wherein the pro-inflammatory activity comprises the production and/or release of a pro-inflammatory mediator.
6. The method of embodiment 5, wherein the pro-inflammatory mediator is a pro-inflammatory cytokine.
7. The method of embodiment 6, wherein the pro-inflammatory cytokine is IL-1β or TNF.
8. The method of any one of embodiments 1 to 7, wherein the therapeutic agent comprises a particle that is capable of being taken up by the antigen-presenting cell.
9. The method of embodiment 8, wherein the particle is a nanoparticle or microparticle.
10. The method of embodiment 8 or embodiment 9, wherein the particle is a liposome, a lipid-based particle, a polymeric particle, an inorganic particle, an inorganic particle coated with polymer or lipid, a micelle, a filomicelle, an exosome, a lipoprotein, a lipid-coated bubble, a polymersome, a niosome, a carbon nanoassembly, a paramagnetic particle, a ferromagnetic particle, a microvesicle, a dendrimer or a hyperbranched polymer.
11. The method of any one of embodiments 1 to 10, wherein the therapeutic agent comprises a nucleic acid molecule from which 6PGD is producible, wherein the nucleic acid molecule comprises, consists or consists essentially of a 6PGD coding sequence.
12. The method of embodiment 11, wherein the nucleic acid molecule comprises mRNA.
13. The method of embodiment 11, wherein the nucleic acid molecule comprises DNA.
14. The method of embodiment 13, wherein the coding sequence is operably connected to a regulatory element that is operable in the antigen-presenting cell.
15. The method of any one of embodiments 1 to 14, wherein the therapeutic agent comprises an antigen-presenting cell-targeting moiety.
16. The method of any one of embodiments 1 to 15, further comprising contacting the antigen-presenting cell with an HDAC7 polypeptide-producing agent (e.g., an HDAC7 polypeptide or a nucleic acid molecule from which an HDAC7 polypeptide is producible).
17. A method for inhibiting or reducing pro-inflammatory activity of an immune cell, the methods comprising, consisting or consisting essentially of contacting the immune cell with a therapeutic agent and an HDAC7 polypeptide-producing agent (e.g., an HDAC7 polypeptide or a nucleic acid molecule from which an HDAC7 polypeptide is producible), wherein the therapeutic agent comprises D-ribulose-5-phosphate (RL5P), a RL5P analog, a RL5P analog-producing agent and/or a RL5P-producing agent.
18. A therapeutic agent comprising, consisting or consisting essentially of D-ribulose-5-phosphate (RL5P), a RL5P analog, a RL5P analog-producing agent and/or a RL5P-producing agent.
19. The therapeutic agent of embodiment 18, wherein the RL5P-producing agent is 6-phosphogluconate dehydrogenase (6PGD) and/or a nucleic acid molecule from which 6PGD is producible.
20. The therapeutic agent of embodiment 18 or embodiment 19, wherein the therapeutic agent comprises a particle that is capable of being taken up by an antigen-presenting cell.
21. The therapeutic agent of embodiment 20, wherein the particle is a nanoparticle or microparticle.
22. The therapeutic agent of embodiment 20 or embodiment 21, wherein the particle is a liposome, a lipid-based particle, a polymeric particle, an inorganic particle, an inorganic particle coated with polymer or lipid, a micelle, a filomicelle, an exosome, a lipoprotein, a lipid-coated bubble, a polymersome, a niosome, a carbon nanoassembly, a paramagnetic particle, a ferromagnetic particle, a microvesicle, a dendrimer or a hyperbranched polymer.
23. The therapeutic agent of any one of embodiments 18 to 22, wherein the therapeutic agent comprises a nucleic acid molecule from which 6PGD is producible, wherein the nucleic acid molecule comprises, consists or consists essentially of a 6PGD coding sequence.
24. The therapeutic agent of embodiment 23, wherein the nucleic acid molecule comprises mRNA.
25. The therapeutic agent of embodiment 23, wherein the nucleic acid molecule comprises DNA.
26. The therapeutic agent of embodiment 25, wherein the coding sequence is operably connected to a regulatory element that is operable in an antigen-presenting cell.
27. The therapeutic agent of any one of embodiments 18 to 26, wherein the therapeutic agent targets an antigen-presenting cell.
28. The therapeutic agent of any one of embodiments 18 to 27, further comprising an antigen-presenting cell-targeting moiety.
29. A therapeutic combination comprising, consisting or consisting essentially of a therapeutic agent and an HDAC7 polypeptide-producing agent(e.g., an HDAC7 polypeptide or a nucleic acid molecule from which an HDAC7 polypeptide is producible), wherein the therapeutic agent comprises D-ribulose-5-phosphate (RL5P), a RL5P analog, a RL5P analog-producing agent and/or a RL5P-producing agent (e.g., 6PGD, or a nucleic acid molecule from which 6PGD is producible).
30. The therapeutic combination of embodiment 29, wherein one or both of the therapeutic agent and the HDAC7 polypeptide-producing agent are comprised or otherwise associated with one or more particles.
31. The therapeutic combination of embodiment 30, wherein the therapeutic agent and the HDAC7 polypeptide-producing agent are comprised or otherwise associated with the same particle or with different particles.
32. The therapeutic combination of embodiment 30 or embodiment 31, wherein the particle or particles are capable of being taken up by an immune cell.
33. The therapeutic combination of any one of embodiments 30 to 32, wherein the particle or particles are nanoparticles or microparticles.
34. The therapeutic agent of any one of embodiments 30 to 33, wherein the particle or particles are selected from liposomes, lipid-based particles, polymeric particles, inorganic particles, inorganic particles coated with polymer or lipid, micelles, filomicelles, exosomes, lipoproteins, lipid-coated bubbles, polymersomes, niosomes, carbon nanoassemblies, paramagnetic particles, ferromagnetic particles, microvesicles, dendrimers and hyperbranched polymers.
35. A pharmaceutical composition comprising the therapeutic agent of any one of embodiments 18 to 28, and a pharmaceutically acceptable carrier, diluent or excipient.
36. A pharmaceutical composition comprising the therapeutic combination of any one of embodiments 29 to 34, and a pharmaceutically acceptable carrier, diluent or excipient.
37. A method for treating, preventing, inhibiting or reducing, or slowing the progression of, inflammation in a subject, the method comprising administering to the subject an effective amount of the therapeutic agent of any one of embodiments 18 to 28, or the therapeutic combination of any one of embodiments 29 to 34, or the pharmaceutical composition of embodiment 35 or embodiment 36.
38. The method of embodiment 37, wherein the inflammation is associated with the presence of an antigen-presenting cell that produces a pro-inflammatory mediator.
39. The method of embodiment 38, wherein the pro-inflammatory mediator is a pro-inflammatory cytokine.
40. The method of embodiment 39, wherein the pro-inflammatory cytokine is IL-1p or TNF.
41. The method of any one of embodiments 37 to 40, wherein the subject has or is at risk of developing an acute inflammatory condition.
42. The method of embodiment 41, wherein the acute inflammatory condition is selected from cytokine release syndrome (CRS) or a cytokine storm, multisystem inflammatory syndrome in children (MIS-C), systemic inflammatory response syndrome (SIRS), acute respiratory distress syndrome (ARDS), severe acute respiratory syndrome (SARS), ischemia, ischemia-reperfusion injuries, hemorrhagic shock, transfusion related acute lung injury (TRALI), pancreatitis, dermatitis, gingivitis, acute meningitis, acute gastritis, acute sarcoidosis, pneumonia, acute allergic rhinitis, glomerulonephritis, acute liver injury, renal failure, acute kidney disease, acute bacterial, viral or fungal infection, acute graft versus host disease, acute gout, acute thyroiditis, nephritis, polyarteritis nodosa, necrotizing enterocolitis, endotoxemia, septicemia, toxic shock syndrome, and acute tissue injury.
43. The method of embodiment 41, wherein the acute inflammatory condition is associated with or results from trauma, burns, massive transfusion, radiation injury, reperfusion injury, traumatic nerve injury, spinal cord injury, aging, chemical exposure, oxidative damage to tissues or cardiopulmonary bypass.
44. The method of embodiment 41, wherein the acute inflammatory condition is associated with a cardiovascular disease.
45. The method of embodiment 44, wherein the cardiovascular disease is selected from myocardial infarction, stroke, vasculitis, microvasculopathy, stable angina pectoris (SAP), unstable angina pectoris (UAP), acute arrhythmia, acute rheumatic heart disease, acute myocarditis, acute pericarditis, acute heart failure (aHF), acute ischemic heart disease (aIHD), sudden cardiac death (SCD), cardiac tamponade, cardiogenic shock, acute coronary artery syndrome and hypertensive crisis.
46. The method of any one of embodiments 37 to 40, wherein the subject has or is at risk of developing a chronic inflammatory condition.
47. The method of embodiment 46, wherein the chronic inflammatory condition is selected from chronic liver disease, inflammatory bowel disease (IBD) (e.g., Crohn's disease or ulcerative colitis), asthma, byssinosis, asbestosis, pneumonitis, pneumonia, chronic meningitis, diabetes, chronic allergic rhinitis, chronic sarcoidosis, chronic kidney disease, chronic thyroiditis, sciatica, pulmonary fibrosis, cystic fibrosis, polymyositis, gout, and chronic obstructive pulmonary disorder (COPD).
48. The method of embodiment 46, wherein the chronic inflammatory condition is associated with an autoimmune disease, a neurodegenerative disease, or a cardiovascular disease.
49. The method of embodiment 48, wherein the autoimmune disease is selected from rheumatoid arthritis, systemic lupus erythematosus (SLE), type 1 diabetes, psoriasis, Graves' disease, colitis, autoimmune encephalitis, autoimmune kidney disease, celiac disease, Sjogren's syndrome, primary biliary cirrhosis, autoimmune hepatitis, Immune Thrombocytic Purpura (ITP), chronic glomerulonephritis, polymyositis, Addison's disease, systemic sclerosis, and graft versus host disease.
50. The method of embodiment 48, wherein the neurodegenerative disease is selected from Alzheimer's disease, amyotrophic lateral sclerosis (ALS), multiple sclerosis (MS), Friedreich ataxia, Huntington's disease, Lewy body disease, Parkinson's disease, Guillain-Barre syndrome, spinal muscular atrophy, Bell's Palsy, spinal cord injury, cerebral aneurysm, epilepsy, and seizures.
51. The method of embodiment 48, wherein the cardiovascular disease is selected from atherosclerosis, coronary artery disease (CAD), chronic rheumatic heart disease, peripheral artery disease, peripheral vascular disease (PAD), congenital heart disease (CHD), sickle cell anemia, congestive heart failure, myocardial ischemia, chronic arrhythmia, cardiomyopathy, and thrombosis.
52. The method of embodiment 46, wherein the chronic inflammatory condition is associated with chronic granulomatous disease (CGD).
53. A method for treating an acute inflammatory condition in a subject, the method comprising administering to the subject an effective amount of the therapeutic agent of any one of embodiments 18 to 28, or the therapeutic combination of any one of embodiments 29 to 34, or the pharmaceutical composition of embodiment 35 or embodiment 36.
54. The method of embodiment 53, wherein the acute inflammatory condition is selected from cytokine release syndrome (CRS) or a cytokine storm, multisystem inflammatory syndrome in children (MIS-C), systemic inflammatory response syndrome (SIRS), acute respiratory distress syndrome (ARDS), severe acute respiratory syndrome (SARS) ischemia, ischemia-reperfusion injuries, hemorrhagic shock, transfusion related acute lung injury (TRALI), pancreatitis, dermatitis, gingivitis, acute meningitis, acute gastritis, acute sarcoidosis, pneumonia, acute allergic rhinitis, glomerulonephritis, acute liver injury, renal failure, acute kidney disease, acute bacterial, viral or fungal infection, acute graft versus host disease, acute gout, acute thyroiditis, nephritis, polyarteritis nodosa, necrotizing enterocolitis, endotoxemia, septicemia, toxic shock syndrome, and acute tissue injury.
55. The method of embodiment 53, wherein the acute inflammatory condition is associated with or results from trauma, burns, massive transfusion, radiation injury, reperfusion injury, traumatic nerve injury, spinal cord injury, aging, chemical exposure, oxidative damage to tissues or cardiopulmonary bypass.
56. The method of embodiment 53, wherein the acute inflammatory condition is associated with a cardiovascular disease.
57. The method of embodiment 56, wherein the cardiovascular disease is selected from myocardial infarction, stroke, vasculitis, microvasculopathy, stable angina pectoris (SAP), unstable angina pectoris (UAP), acute arrhythmia, acute rheumatic heart disease, acute myocarditis, acute pericarditis, acute heart failure (aHF), acute ischemic heart disease (aIHD), sudden cardiac death (SCD), cardiac tamponade, cardiogenic shock, acute coronary artery syndrome, and hypertensive crisis.
58. A method for treating a chronic inflammatory condition in a subject, the method comprising administering to the subject an effective amount of the therapeutic agent of any one of embodiments 18 to 28, or the therapeutic combination of any one of embodiments 29 to 34, or the pharmaceutical composition of embodiment 35 or embodiment 36.
59. The method of embodiment 58, wherein the chronic inflammatory condition is selected from chronic liver disease, chronic graft versus host disease, inflammatory bowel disease (IBD) (e.g., Crohn's disease or ulcerative colitis), asthma, byssinosis, asbestosis, pneumonitis, pneumonia, chronic meningitis, diabetes, chronic allergic rhinitis, chronic sarcoidosis, chronic kidney disease, chronic thyroiditis, sciatica, pulmonary fibrosis, cystic fibrosis, polymyositis, gout, and chronic obstructive pulmonary disorder (COPD).
60. The method of embodiment 58, wherein the chronic inflammatory condition is an autoimmune disease, a neurodegenerative disease, or a cardiovascular disease.
61. The method of embodiment 60, wherein the autoimmune disease is selected from rheumatoid arthritis, systemic lupus erythematosus (SLE), type 1 diabetes, psoriasis, Graves' disease, colitis, autoimmune encephalitis, autoimmune kidney disease, celiac disease, Sjogren's syndrome, primary biliary cirrhosis, autoimmune hepatitis, Immune Thrombocytic Purpura (ITP), chronic glomerulonephritis, polymyositis, Addison's disease, systemic sclerosis, and graft versus host disease.
62. The method of embodiment 60, wherein the neurodegenerative disease selected from Alzheimer's disease, amyotrophic lateral sclerosis (ALS), multiple sclerosis (MS), Friedreich ataxia, Huntington's disease, Lewy body disease, Parkinson's disease, Guillain-Barre syndrome, spinal muscular atrophy, Bell's Palsy, spinal cord injury, cerebral aneurysm, epilepsy, and seizures.
63. The method of embodiment 60, the cardiovascular disease is selected from atherosclerosis, coronary artery disease (CAD), chronic rheumatic heart disease, peripheral artery disease, peripheral vascular disease (PAD), congenital heart disease (CHD), sickle cell anemia, congestive heart failure, myocardial ischemia, chronic arrhythmia, cardiomyopathy, and thrombosis.
64. The method of embodiment 58, wherein the chronic inflammatory condition is chronic granulomatous disease (CGD).
65. Use of the therapeutic agent of any one of embodiments 18 to 28, or the therapeutic combination of any one of embodiments 29 to 34, or the pharmaceutical composition of embodiment 35 or embodiment 36 in the manufacture of a medicament for inhibiting or reducing pro-inflammatory activity of an antigen-presenting cell, or for treating an inflammatory response, or for treating an acute inflammatory condition, or treating a chronic inflammatory condition.
66. Use of the therapeutic combination of any one of embodiments 29 to 34, or the pharmaceutical composition of embodiment 36 in the manufacture of a medicament for inhibiting or reducing pro-inflammatory activity of an immune cell, or for treating an inflammatory response, or for treating an acute inflammatory condition, or treating a chronic inflammatory condition.
67. The method of any one of embodiments 1 to 17 and 37 to 64, or the therapeutic agent of any one of embodiments 18 to 28, or the therapeutic combination of any one of embodiments 29 to 34, or the pharmaceutic composition of embodiment 35 or embodiment 36, or the use of embodiment 65, wherein the therapeutic agent targets an antigen-presenting cell.

In order that the disclosure may be readily understood and put into practical effect, particular preferred embodiments will now be described by way of the following non-limiting examples.

### EXPERIMENTAL

### Investigating the Inhibition of the 6PGD-RL5P Axis on the Inflammatory Response in Macrophages

6PGD, along with glucose-6-phosphate dehydrogenase (G6PD), is responsible for generating NADPH that is subsequently used by the phagocyte NADPH oxidase to generate ROS for antimicrobial host defense (Jin and Zhou, 2019).

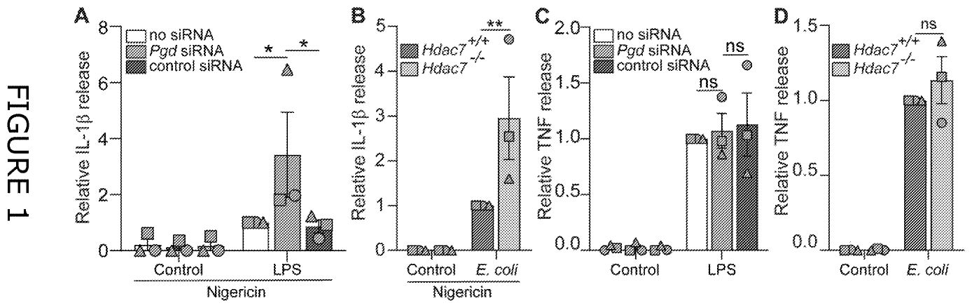

As disclosed herein, the present inventors found that either reducing the expression of 6PGD by siRNA (i.e., Pgd silencing) or genetic targeting of HDAC7 (knock out of Hdac7-Hdac7^{-/-}) to suppress 6PGD enzyme activation in bone marrow-derived macrophages (BMM), resulted in increased IL-1β production in activated macrophages i.e., the macrophages were less able to suppress the inflammatory pathway (FIG. 1A-B). These effects were at least partly selective to IL-1β, as antagonizing 6PGD expression or function or targeting HDAC7 did not affect inducible TNF production in BMM (FIG. 1C-D).

### Investigating the Overexpression of 6Pgd on the Inflammatory Response in Macrophages

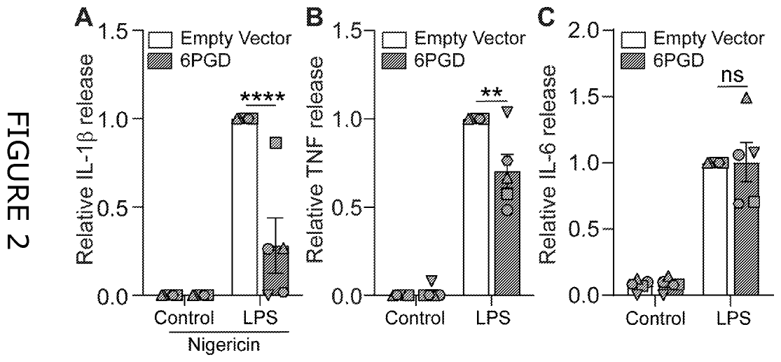

Through gain-of-function studies, the present inventors found that over-expression of 6PGD attenuates both inducible IL-1β and TNF but does not affect IL-6 in BMM (FIG. 2A-C). Note that (a) agonizing this pathway inhibits TNF in BMM, whereas blocking the pathway does not increase TNF in these cells (see above); and (b) the effect on TNF is much more striking for human macrophages than for mouse macrophages (see below).

Consistent with the Pgd silencing data described above, ectopic expression of 6PGD in BMM attenuated the inducible IL-1β response (FIG. 2A). In this gain-of-function approach, 6PGD overexpression also significantly reduced LPS-induced TNF (FIG. 2B). However, the effect of 6PGD overexpression was selective, since LPS-induced IL-6 was unaffected (FIG. 2C). These experiments thus revealed that 6PGD serves a key role during innate immune responses to threats that activate the 6PGD-5RLP pathway of the PPP, limiting inflammatory responses to enable cells to focus their resources on bacterial killing.

### Investigating the Effect of Treatment with RL5P on Macrophage Inflammatory Response

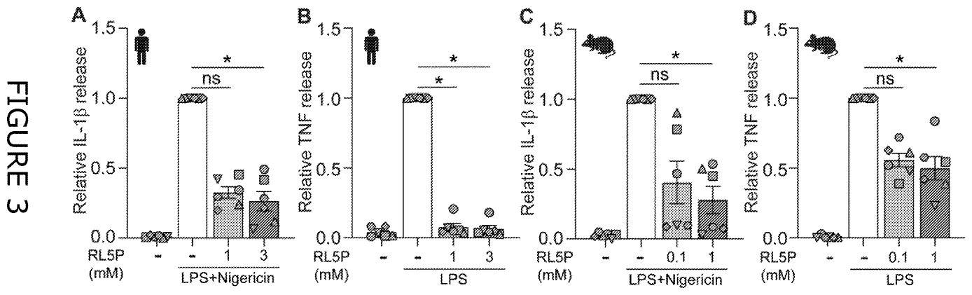

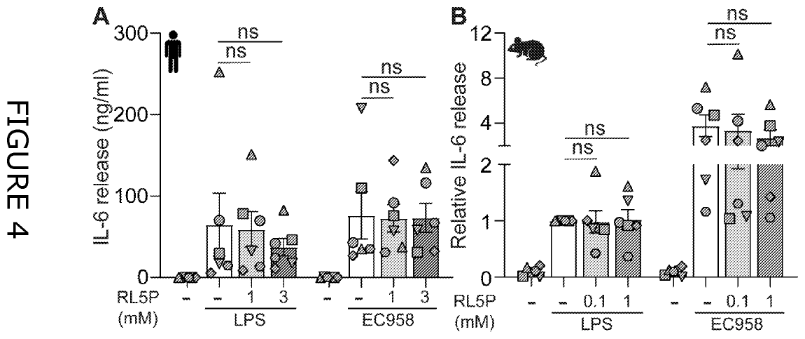

Similar to the results of overexpression of 6PGD described above, the present inventors found that treatment with RL5P (a biochemical enzyme product of 6PGD), suppresses LPS-induced IL-1β and TNF in both HMDM (FIGS. 3A-B) and BMM (FIGS. 3C-D) as discussed below, without affecting LPS-induced IL-6 production (FIGS. 4A-B).

First, the present inventors examined the effects of RL5P, the enzymatic product of 6PGD, on macrophage inflammatory responses. Remarkably, RL5P treatment suppressed LPS-inducible IL-1β production in human and mouse macrophages (FIGS. 3A and 3C). In BMM, and to a lesser extent HMDM, this effect was much less pronounced in cells responding to Escherichia coli. RL5P treatment also reduced inducible TNF production in macrophages (FIGS. 3B and 3D). In this case, RL5P dramatically reduced secreted TNF levels in HMDM, whereas the effect on BMM was modest. These phenotypes are comparable to the effect of 6PGD over-expression on LPS-induced TNF in BMM (FIG. 2B). However, the anti-inflammatory effects of RL5P in both HMDM and BMM were selective, since this metabolite did not affect inducible IL-6 production in cells responding to either distal (i.e., LPS) or proximal (i.e., E. coli) threats (FIG. 4A-B). Thus, the present inventors found that supplementation with RL5P enables suppression of specific inflammatory responses in macrophages responding to LPS-induced threats.

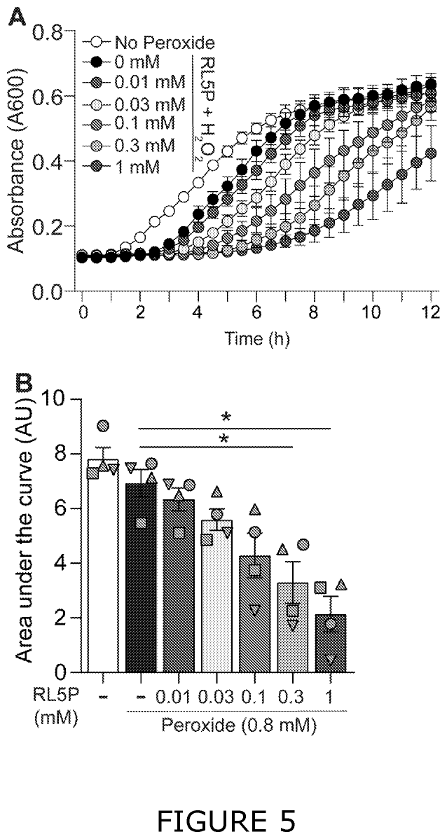

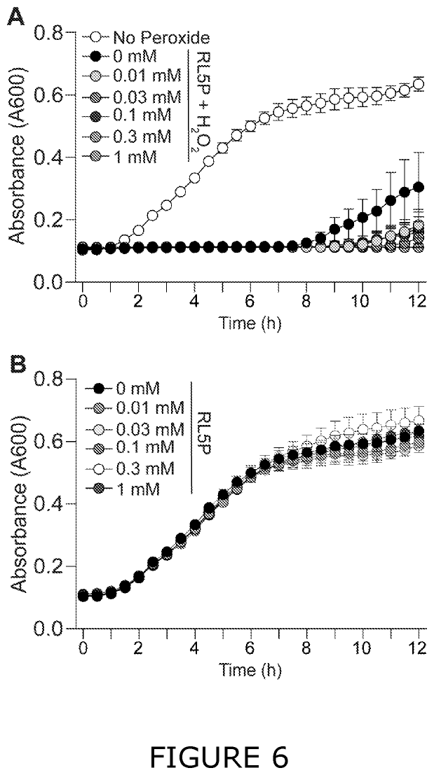

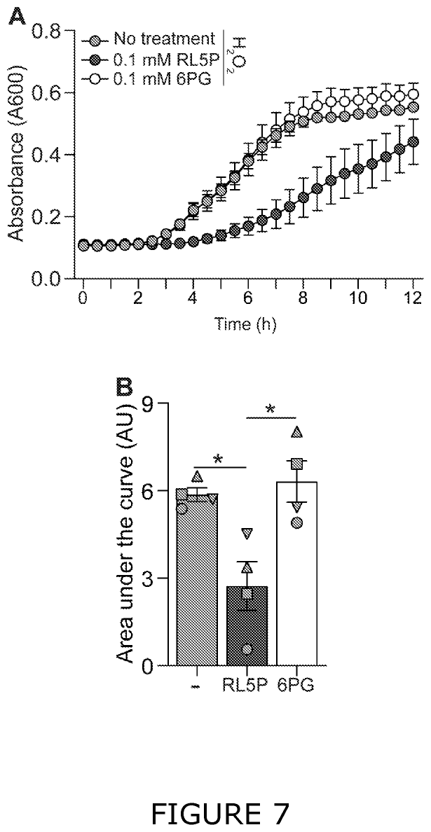

Next, given the immunomodulatory properties of RL5P that were identified, as well as the fact that the RL5P producing enzyme 6PGD can physically interact with and regulate the phagocyte oxidase (Paclet et al., 2007. FASEB J. 21:1244-1255; Baillet et al., 2011. FASEB J. 25:2333-2343), the present inventors considered that RL5P may also contribute to oxidative stress-mediated antimicrobial defense against phagocytosed bacteria. To test this model, the present inventors examined the effect of increasing RL5P concentrations on the growth of UPEC under conditions of oxidative stress. When UPEC were grown in a concentration of hydrogen peroxide that had a minimal effect on bacterial growth (0.8 mM H₂O₂), RL5P significantly impaired E. coli growth in a dose-dependent manner (FIG. 5A, B). This effect was also apparent for UPEC cultured with 1 mM H₂O₂ that dramatically impaired bacterial growth (FIG. 6A). Importantly, RL5P did not affect UPEC growth in the absence of oxidative stress (FIG. 6B). Furthermore, in contrast to the findings with RL5P, the upstream PPP metabolite and 6PGD substrate, 6-phosphogluconate (6PG), did not affect UPEC growth in the presence of H₂O₂(FIG. 7A, B). These data support a role for RL5P in oxidative stress-mediated antimicrobial responses in macrophages. In summary, the present inventors have identified both antimicrobial and anti-inflammatory properties of the PPP metabolite RL5P, opening up potential immune modulation strategies for applications in inflammatory and/or infectious diseases.

### EXPERIMENTAL METHODS

### Ethics Statement

All studies involving animals were reviewed and approved by the appropriate University of Queensland animal ethics committee. Human peripheral blood was collected from healthy donors following informed consent, under approval by the University of Queensland Institutional Human Research Ethics Committee.

### Animals

All studies involving animals were approved by the relevant animal ethics committee at The University of Queensland. Male and female C57BL/6J mice of 8-12 weeks age were obtained from an in-house breeding colony within the Queensland Biosciences Precinct animal house (The University of Queensland) or from Ozgene (Murdoch, WA). Mice were housed on a 12 h controlled day/night cycle with food and water available ad libitum in the Institute for Molecular Bioscience animal house for 7 days. Animals were monitored during and after experiments in accordance with the standards in ethics approval.

### Bacterial Culture

All bacterial strains were cultured at 37° C. on solid or liquid Luria-Bertani (LB) medium. The non-pathogenic E. coli K12 strain MG1655 (Blattner et al., 1997. Science. 277:1453-1462) and EC958, a representative strain of the globally-disseminated multidrug-resistant ST131 clone isolated from the urine of a patient with a urinary tract infection (Totsika et al., 2011, PloS one. 6:e26578-e26578; Petty et al., 2014, Proc Natl Acad Sci USA. 111:5694-5699), were used in this study. To assess the effect of RL5P and 6PG on UPEC growth, E. coli strain EC958 was incubated with the indicated concentrations of these metabolites with or without the indicated concentrations of H₂O₂ for 12 h at 37° C. Bacterial growth was assessed by monitoring OD₆₀₀ using a POLARstar Omega (BMG Labtech) at 20-30 min intervals.

### Mammalian Cell Culture

BMM were generated from indicated strains of mice, as previously described (Das Gupta et al., 2020, Cell Rep. 30:2712-2728.e2718). Bone marrow cells were cultured for 6 days in RPMI-1640 media (Thermo Fisher Scientific) supplemented with 10% FCS, 50 U/mL penicillin, 50 μg/mL streptomycin and 2 mM L-glutamine, in the presence of recombinant human colony-stimulating factor 1 (CSF-1), at either 1×10⁴ U/mL (Chiron) or 150 ng/mL (The University of Queensland Protein Expression Facility). On day 6, BMM were harvested and replated at an appropriate density with fresh CSF-1, with cells being used in experiments on the following day. CD 14+ human monocytes, which were purified from buffy coats provided by the Australian Red Cross Blood Service with approval from the University of Queensland Human ethics committee (2013001519), were differentiated into human monocyte-derived macrophages (HMDM) by culturing for 7 days in IMDM containing 10% FCS, 50 U/mL penicillin, 50 μg/mL streptomycin, 2 mM L-glutamine and recombinant human CSF-1 (as above). No donor identity was provided, and it is presumed that there were equal or similar numbers of male and female donors. PlatE cells, used for generating retrovirus as well has HEK293T cells, were cultured in DMEM media supplemented with 10% FCS, 50 U/mL penicillin, 50 μg/mL streptomycin. For all infection assays (see below), cells were cultured in IMDM (Thermo Fisher Scientific) supplemented with 10% FCS. All cells were cultured at 37° C. and 5% CO₂, unless otherwise indicated.

### Gene Silencing

To silence Pgd, BMM incubated at 2.5×10⁶ cells/cuvette in 350 μl complete media supplemented with 10 mM HEPES and indicated siRNAs (Life Technologies) at a final concentration of 1 μM. Cells were electroporated at 260V, 1000 μF and ∞Ω (Bio-Rad), rested for 5 minutes, then plated overnight at 37° C. At 24 h post-electroporation, cells were treated with LPS for 4 h, followed by an additional 1 h of Nigericin treatment. Supernatants were then assessed for cytokines by ELISA.

### Retroviral Transduction

Retroviral overexpression of target genes in primary macrophages was performed as previously described (Das Gupta et al., 2020, Cell Rep. 30:2712-2728.e2718). Briefly, 2×10⁶ PlatE cells were transfected with 40 μg of the respective retroviral expression vectors using Lipofectamine 2000 (Thermo Fisher Scientific). At 24 h post-transfection, media were replaced and cells were incubated at 32° C. for 48 h for optimal virus production. Viral supernatants were filtered through a 0.45 μM Millex-HV PVDF syringe filter (Merck) and supplemented with 1M HEPES, 10 μg/mL polybrene and CSF-1. Bone marrow progenitors treated with CSF-1 for 2 days were spin-infected (1000 g, 2 h, 35° C.) with viral supernatants in 6-well non-tissue culture plates. The cells were immediately supplemented with complete RPMI media, then incubated at 37° C. for 48 h. At 48 h post-transduction, cells were supplemented with complete RPMI media. On day 6 of macrophage differentiation, adherent cells were collected and plated for subsequent experiments.

### Gene Expression Analysis

Total RNA was extracted using RNA purification kits (Qiagen), as per the manufacturer's instructions. RNA was reverse transcribed to cDNA using Superscript III (Invitrogen) and oligo dT. Levels of specific mRNAs were quantified by qPCR using SyBR Green-PCR mix (Invitrogen) in the Applied Biosystems Viia 7 RT-PCR system. Appropriate negative controls with no Superscript III were included for all experiments. Data were expressed relative to the housekeeping gene hypoxanthine phosphoribosyltransferase (HPRT, human) using the ΔCt method (Livak and Schmittgen, 2001).

### Cytokine Analysis

BMM (5×10⁵ cells/mL) or HMDM (5×10⁵ cells/mL) were treated as indicated in individual figure legends. Supernatants were collected and assessed for secreted TNF. To assess secreted IL-1β by ELISA, cells were further treated with Nigericin for 1 h (2 h for HMDMs). Supernatants were collected and analyzed for levels of IL-1β. For experiments comparing macrophages derived from mice of different genotypes, MTT assays were performed in parallel to control for any differences in plating densities.

### Immunoblotting

BMMs transfected with indicated amounts of mRNA or HEK293 cells transfected with plasmid DNA were lysed in RIPA buffer, containing a cocktail of IX protease inhibitors (Roche) and IX PhosSTOP phosphatase inhibitors (Sigma) at 24 h post-transfection. Proteins from whole cell lysates were immunoblotted for V5-tagged 6PGD, V5-tagged HDAC7 or Flag-tagged 6PDG.

### Quantification and Statistical Analysis

Statistical analyses were performed on data combined from three or more independent experiments for cell culture experiments (taking averages from replicates within each experiment). Statistical analyses were performed using Prism 8 software (Graph-Pad) with error bars indicating the standard error of the mean (SEM) or inter-quartile range. Data were analyzed for normality using the Shapiro-Wilk normality test. Statistical analyses on normally distributed data sets were performed using parametric tests (t-test or ANOVA), whereas data sets with non-normal distributions were analyzed using nonparametric tests (Mann-Whitney t-test, Kruskal-Wallis test or Friedman test). For experiments analyzing matched samples or data, repeated measures (RM) ANOVA was performed. For data sets with three or more variables, a one-way or two-way analysis of variance (ANOVA) was performed followed by Tukey's, Dunn's, Dunnett's or Sidak's multiple comparison test. For data with two variables, unpaired t-tests were performed, with appropriate corrections (Giesser Greenhouse correction for ANOVA, and Welch's correction for t-test). Statistical tests used for individual experiments are described in the figure legends. Differences with confidence values of 95% (P<0.05) were considered statistically significant.

In order that the disclosure may be readily understood and put into practical effect, particular preferred embodiments will now be described by way of the following non-limiting examples.

### EXAMPLES

### Example 1

### Delivery of mRNA Encoding GFP to Mouse Bone Marrow-Derived Macrophages (BMM) Using the Neon Gold Transfection System as Proof-of-Concept for Target Expression by mRNA Delivery

Isolation and Differentiation of Mouse Bone Marrow Hematopoietic Stem Cells into BMM

Age-appropriate mice (8-12 weeks, either sex) were sacrificed using CO₂ in a chamber with a fill rate of 30% of the chamber volume per minute. Once culled, the femur and the tibia from the two limbs were removed. Using scissors and scalpels, the muscles were removed from around the two sets of bones, followed by dislocating the bones by pulling them apart. The ends of the bones were cut and using a needle (>23G), 4 mL complete RPMI media was flushed through the bone to collect the hematopoietic stem cells. The cells from all the four bones were centrifuged (500 g, 5 min, RT), resuspended in complete RPMI media and plated onto square sterilin dishes allowing them to differentiate into macrophages in the presence of recombinant CSF-1 (150 ng/mL) for 6 days. On average, cells from four bones were plated onto 8 sterilin dishes. Cells were topped up with fresh complete RPMI and CSF-1 on day 4 of macrophage differentiation. On day 6, the media was removed, after which the cells were lifted using PBS, counted and plated.

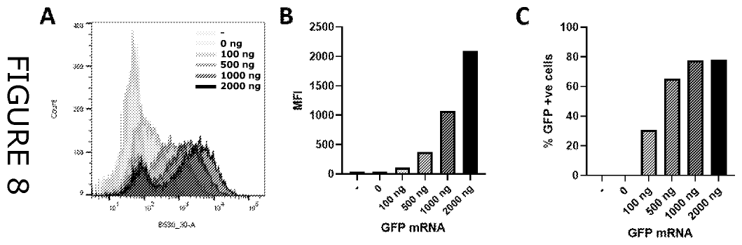

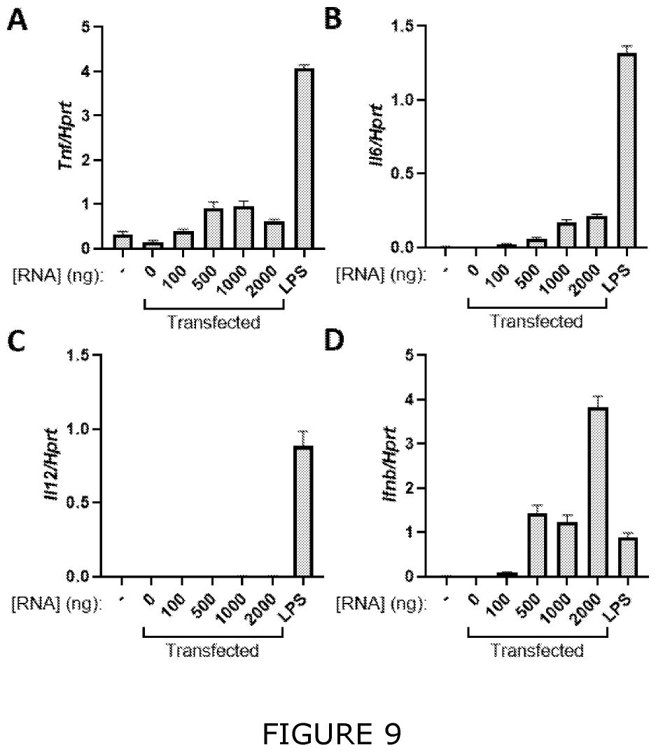

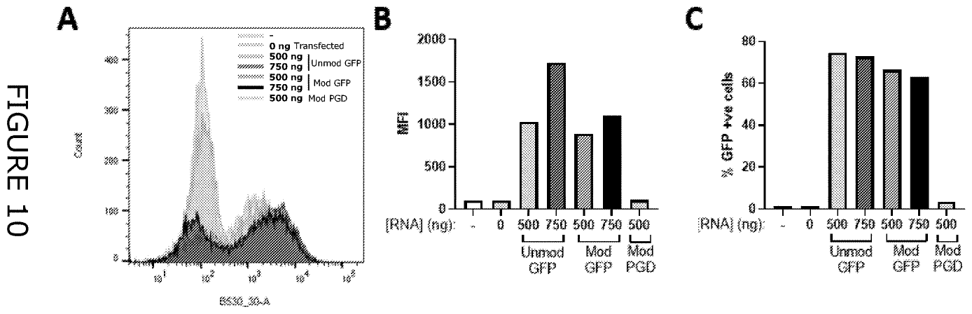

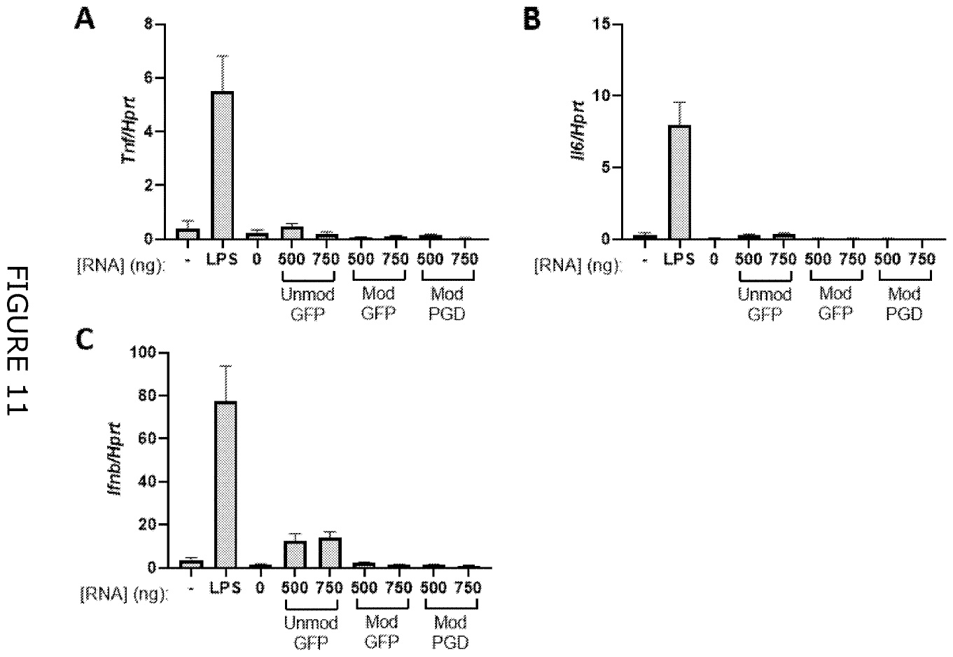

Mouse BMM cells were harvested at day 6 of differentiation and washed with PBS. For each condition, 2×10⁶ cells were resuspended in 100 μL Buffer R from the Neon transfection system kit (Thermo Fisher Scientific). GFP mRNA is diluted appropriately to a final volume of 10 μL in nuclease-free water and mixed with the cell suspension. Cells were transfected using the Neon transfection system with voltage 1400 V, width 20 ms and 2 pulses. Cells were immediately plated on 12-well plates in CSF-1-containing media (no antibiotics). Transfection efficiency was estimated at 24 h post-transfection by lifting the cells in TrypIE Express and measuring GFP levels using flow cytometry (FIG. 8). RNA was also extracted from the cells at 4 h post-transfection, as well as BMM treated with 10 ng/mL LPS for 4 h as a positive control, to measure the expression of inflammatory genes such as Tnf, Il6, Il12b and Ifnb, by RT-qPCR (FIG. 9). Following the above protocol, cells were transfected with unmodified mRNA encoding GFP, alongside GFP and 6PGD mRNA with 5-methoxyuridine modification. Transfection efficiency was estimated at 24 h post-transfection by measuring GFP levels using flow cytometry (FIG. 10) while RNA was extracted from the cells at 4 h post-transfection to measure the expression of inflammatory genes such as Tnf, Il6 and Ifnb, by RT-qPCR (FIG. 11).

In summary, the present inventors have identified that primary mouse macrophages can robustly express a protein of interest through delivery of mRNA encoding that protein, with minimal effects on inflammatory genes, except Ifnb1. Incorporation of 5-methoxyuridine modifications to mRNA prevents the inducible expression of Ifnb mRNA.

### Example 2

### Delivery of mRNA Encoding GFP to Mouse BMM Using Nanoparticles

Mouse BMM cells were prepared in accordance with the method described above in Example 1. The nanoparticles were Dendritic Mesoporous Organosilica Nanoparticles (DMONs), and were prepared according to Example 7 below.

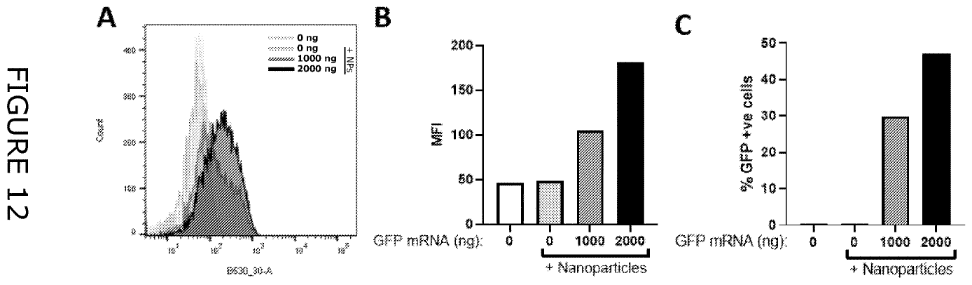

Mouse BMM cells were harvested at day 6 of differentiation and seeded on a 12 well plate overnight. On the following day, nanoparticles were vortexed for 30 sec before incubation with mRNA (40 μg nanoparticles per 1 μg mRNA) for 30 min at room temperature. Nanoparticle and mRNA mixture was added dropwise to cells in serum-free media for at least 4 h. Media was then supplemented with serum and incubated for a further 20 h. Transfection efficiency was estimated at 24 h post-transfection by lifting the cells in TrypIE Express and measuring GFP levels using flow cytometry (FIG. 12).

In summary, the present inventors have shown that nanoparticles can be used for successful delivery of mRNA encoding GFP to BMM and expression of the mRNA in BMM.

### Example 3

### Delivery of 6PDG-mRNA Nanoparticles to Cultured Primary Mouse Macrophages

Transfection of Mouse BMM with 6PGD mRNA Using Neon Transfection System as Proof-of-Concept for Target Expression

Mouse BMM cells were prepared in accordance with the method described above in Example 1. The nanoparticles were DMONs, and were prepared according to Example 7 below.

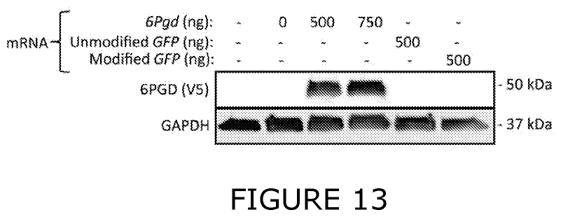

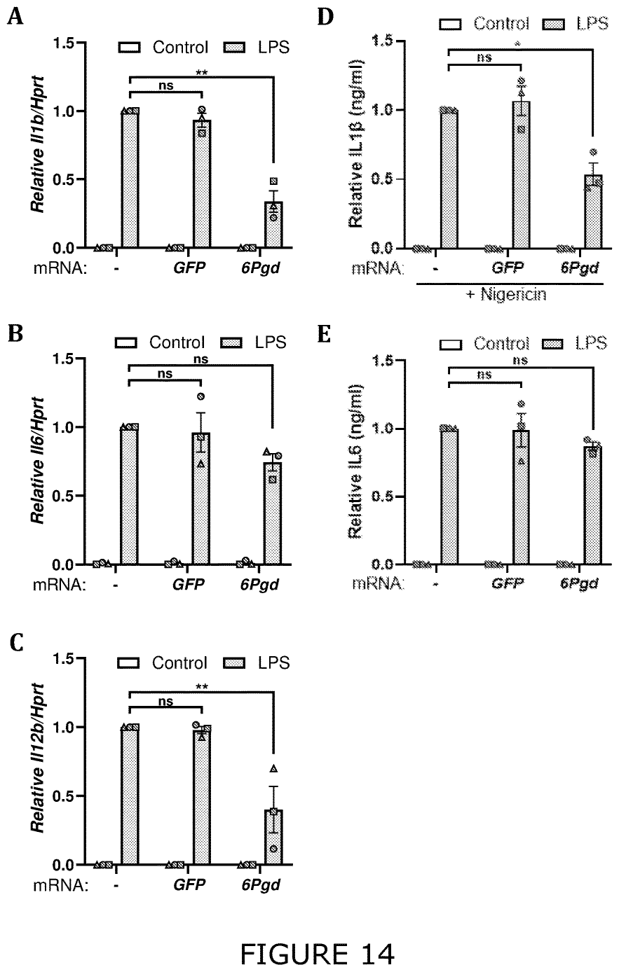

Cells were harvested at day 6 of differentiation and washed with PBS. For each condition, 2×10⁶ cells were resuspended in 100 μL Buffer R from the Neon transfection system kit (Thermo Fisher Scientific). 5-methoxyuridine modified GFP mRNA (GFP), 5-methoxyuridine modified 6PGD mRNA (PGD) or vehicle (0) was diluted appropriately to a final volume of 10 μL in nuclease-free water and mixed with the cell suspension. Cells were transfected using the Neon transfection system with voltage 1400 V, width 20 ms and 2 pulses. Cells were immediately plated on 12-well plates in CSF-1-containing media (no antibiotics). Protein expression from cell lysates was assessed at 24 h post-transfection via western blot (FIG. 13). At 24 h, transfected and untransfected cells (control) were then treated±LPS (1 ng/mL) for 4 h. RNA was extracted and mRNA levels of (A) Il1b, (B) Il6 and (C) Il12b were quantified using RT-qPCR and normalized to the housekeeping gene Hprt (FIG. 14). Secretion levels of IL-1β (D) and IL6 (E) from cell culture supernatants were measured using ELISA.

As such, the present inventors have shown 6PGD protein can be successfully expressed in mouse BMM through delivery of 6PGD-encoding mRNA, with ectopic expression of 6PGD protein selectively suppressing macrophage inflammatory genes (suppression of Il1b, Il12b but not 116) and protein secretion of IL-1 S.

### Example 4

### Delivery of 6PDG-mRNA Nanoparticles to Cultured Primary Human Macrophages

Isolation and Differentiation of Human CD14+ Monocytes from Buffy Coats into Primary Monocyte Derived Macrophages

Approximately 50 mL buffy coat is diluted to 120 mL using saline. The saline buffy coat mixture (30 mL) is gently laid over Ficoll-Paque (12.5 mL in 50 mL falcon tubes). The tubes are centrifuged for 45 min at 200 g at room temperature with brakes set to low. Once the ficoll gradient separation is complete, the top serum layer is gently removed and the middle fluffy white layer containing the white blood cells is collected in a new 50 mL falcon tube (the lower RBC layer is discarded). The white blood cells are washed twice with saline (600 g, 10 min, 10° C., brakes set to normal) and finally resuspended in 10 mL MACS buffer (DPBS containing 2 mM EDTA and 0.5% FBS). The white blood cells are mixed with 300 μL CD14⁺ microbeads and incubated for 1 h at 4° C. under shaking conditions. The mixture is then topped with equal amounts of MACS buffer and centrifuged at 400 g for 5 min at 4° C. LS columns (Miltenyl) are calibrated with 3 mL MACS buffer (2-3 columns per donor). Pelleted leukocytes are resuspended in 15 mL MACS buffer and passed through cell sieve, adding extra MACs buffer if necessary to wash through. The leukocytes are then passed through the LS columns and washed twice with 5 mL MACS buffer. Positively selected cells are eluted from the columns using 5 mL MACS buffer by removing column from magnetic field and applying plunger force. Monocytes are counted and plated at a density of 15 million monocytes in 10 mL IMDM in 10 cm cell culture grade petri dishes and differentiated into primary macrophages using CSF-1 (150 ng/mL) for 6 days. On day 4 of macrophage differentiation, the cells are topped with 5 mL IMDM containing CSF-1. Post differentiation, the supernatants are removed and the adhered cells are washed once with PBS before they are lifted off, counted and plated for appropriate experiments.

Nanoparticles (DMONs prepared according to Example 7 below) containing vehicle control, 6PDG or GFP control mRNA (500 ng to 2000 ng) are incubated with cultured primary mouse and human macrophages, for up to 24 h. Cells are stimulated±LPS for 4 h or 24 h, then ±Nigericin for 1 h or are left untreated. Culture supernatants are harvested at appropriate time points and assessed for IL-1β, TNF, and IL-6 cytokines using ELISA. RNA is also extracted from cells at appropriate time points and reverse transcribed into cDNA to measure the expression of 6pgd mRNA, as well as those of a panel of inflammatory mediators (Tnf, Il1b, Il6, Il12b) using qPCR.

Expression of 6PGD, but not the GFP control, is expected to suppress LPS-induced expression and release of a subset of inflammatory mediators (IL-1β, TNF) but not others (IL-6, IL-12p40).

### Example 5

### In Vivo Delivery of mRNA in LPS Shock Model

For in vivo experiments, 8-12 week old male C57BL/6J mice were injected intraperitoneally with 10 μg of GFP- or 6PGD-encoding mRNA corresponding to SEQ ID No. 3 (with linker and V5 tag) formulated with silica-based nanoparticles (SNPs) with a uniform particle size on 370 nm, a pore size of 10-20 nm and a spike size of 40-50 nm (prepared according to the methods described in Cheng et al. Sci Adv. 2023; 9(40):eadi7502, and PCT/AU2016/050283) or lipid nanoparticles (LNPs) (prepared according to the Pfizer-BioNTech formulation in Table 1 of Schoenmaker et al. Int J Pharm. 2021; 15:601) for 19 h. Mice were then injected intraperitoneally with LPS (2 mg/kg).Total body weight was recorded prior to LPS injection and again at 6 h post-injection. Mice were weighed and euthanized at 6 h post-injection and tissues were collected in RLT lysis buffer. Tissues were homogenized and RNA extracted using ISOLATE II RNA Mini kit (BioLine) as per the manufacturer's instructions, RNA was reverse transcribed to cDNA using Superscript III (Invitrogen) and random primers. Levels of Il1b were quantified by qPCR using SyBR Green-PCR mix (Invitrogen) in the Applied Biosystems Viia 7 RT-PCR system. Appropriate negative controls with no Superscript III were included for all experiments. Data were expressed relative to the housekeeping gene hypoxanthine phosphoribosyl transferase (Hprt, mouse).

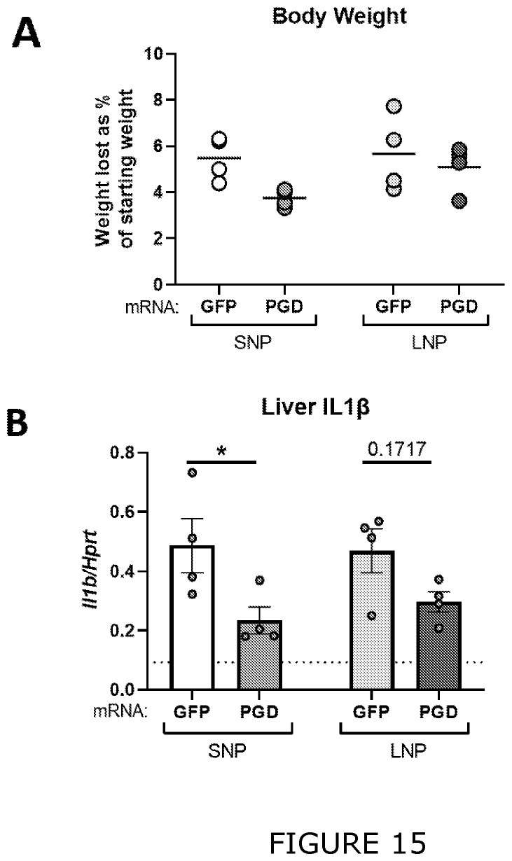

FIG. 15A shows that nanoparticle delivery of 6PGD mRNA reduces loss of body weight in mice challenged with LPS, when compared with GFP mRNA. Silica-based nanoparticles formulated with 6PGD mRNA show greater effect than the lipid nanoparticle formulation.

FIG. 15B shows that nanoparticle delivery of 6PGD mRNA significantly reduces mRNA expression of the inflammatory mediator IL-1β in livers of mice challenged with LPS, when compared with GFP mRNA. Both lipid- and silica-based nanoparticles formulated with 6PGD mRNA reduced IL-1β mRNA expression in livers of mice challenged with LPS, as compared to the GFP-expressing control. Notably, silica-based nanoparticles show a greater effect than the lipid nanoparticle formulation.

### Example 6

### Protein Expression for 6PGD vs GFP Control in Liver with SNPs vs LNPs

Immunofluorescence was performed on 4 μm paraffin-embedded sections from mice liver from Example 5. The sections were de-waxed using xylene and rehydrated in a gradient of ethanol (100, 95, 80 and 70%). Antigen unmasking was performed by boiling the slides in sodium citrate buffer (10 mM Tri-sodium citrate, 0.05% Tween, pH 6.0) for 10 min. Slides were then blocked in blocking buffer (5% FCS, 0.3% Triton X-100 in PBS). Incubations in primary antibodies was performed overnight using mouse anti-V5 (1 in 1000 dilution in blocking buffer) and rat anti-F4/80 (1 in 400 dilution in blocking buffer), followed by incubation with secondary antibodies (anti-mouse Alexa −488 and anti-rat Alexa −647) for 1 h. Slides were subsequently stained with DAPI for 10 min, after which they were mounted using IMBiol mounting media (IMB microscopy) and sealed using nail polish. Images were captured at using the Carl Zeiss Meta Inverted LSM 510 microscope (Carl Zeiss) and processed using ImageJ.

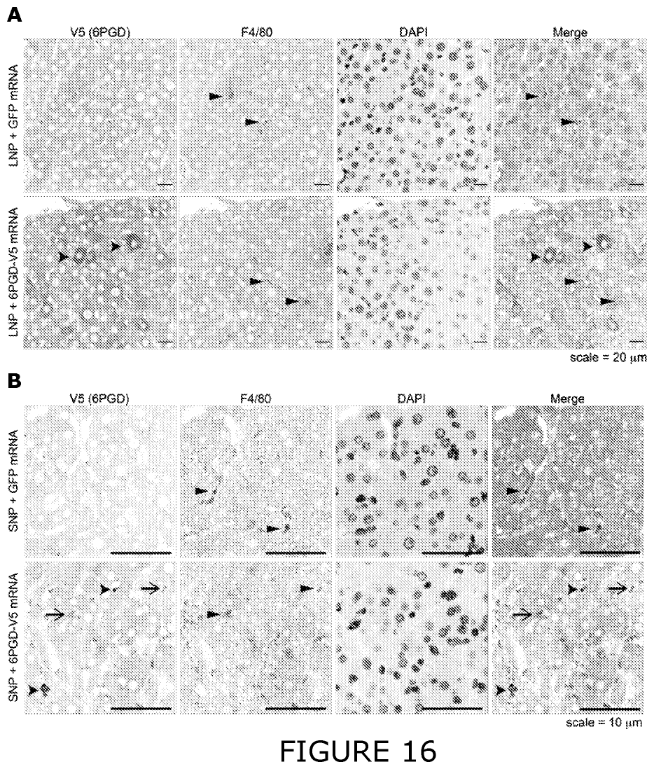

FIG. 16A shows the presence of 6PGD (V5) in the livers of mice injected with 6PGD mRNA+LNP, while it was not observed in the livers of mice injected with GFP mRNA+SNP. 6PGD (V5) was detected in the liver hepatocytes and showed no co-localization with the macrophage stain F4/80.

FIG. 16B show the presence of 6PGD (V5) in the livers of mice injected with 6PGD mRNA+SNP, while it was not observed in the livers of mice injected with GFP mRNA+SNP. Furthermore, the cells that tested positive for V5 (6PGD) staining exhibited partial co-localization with the macrophage marker F4/80, as indicated by the arrows. However, some cells expressing V5 did not show co-localization with F4/80 staining, as indicated by the curved arrows. Notably, 6PGD (V5) was not detected in liver hepatocytes.

### Example 7

### Synthesis of Dendritic Mesoporous Organosilica Nanoparticles (DMONs)

In a typical synthesis, 34.0 mg of triethanolamine was dissolved in 12.50 mL of deionized water and stirred at room temperature for 0.5 h. Then, 190.0 mg of cetyltrimethylammonium bromide and 24.6 mg of sodium heptafluorobutyrate were added to the above solution for further stirring at 40° C. After 1 h, the premixed inorganic precursor of 0.99 mL of tetraethyl orthosilicate and 0.319 mL tetrabutyl orthosilicate was added and stirred at 40° C. for 0.5 h, followed by the addition of 0.60 mL of 1,4-bis(triethoxysilyl)-propane tetrasulfide (BTES) for further stirring for 24 h. The solid precipitates were collected by centrifugation, thorough washing with ethanol for three times, and dried in the vacuum oven overnight. Surfactants were removed through acid extraction with HCl and ethanol (volume ratio=1:20) for 6 h at 60° C. The extraction treatment was repeated 3 times. The final product was denoted DMONs-40-R0.2.

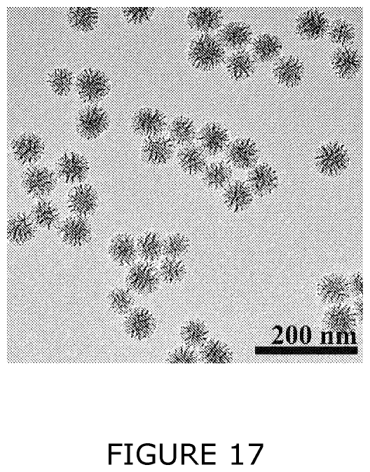

Transmission electron microscopy (TEM) revealed that the DMONs so produced exhibit a large pore dendritic structure, with an average particle size of 50 nm (FIG. 17).

### Example 8

### Polyethyleneimine (PEI) Modification I

30 mg of DMONs-40-R0.2 were dissolved through sonication in 10 mL pH=10 water (adjusted by 30% ammonia solution) and then mixed with 10 mL of 56 mM Sodium 3-(trihydroxysilyl) propylmethylphosphonate (THPMP) solution with stirring at 40° C. After 2 h, samples were collected through centrifugation and washed with deionized water twice. The product was then resuspended in 15 mL of carbonate buffer containing 30 mg of poly(ethyleneimine (PEI 10 K) with further stirring for 2 h at room temperature. The final product was collected through centrifugation, washing with deionized water, and freezing drying. The final product was denoted as DMONs-40-R0.2-PEI.

### Example 9

### PEI Modification II

100 mg of SNPs were dissolved through sonication in 30 ml toluene, refluxed at 70° C. for 15 min, and then 1.5 mL of 3-glyciodoxypropyl trimethoxysilane was added for refluxing overnight. The samples were collected through centrifugation and washed with methanol and toluene twice. After freeze-drying, the dry powder was then resuspended in 50 mL of carbonate buffer containing 250 mg of poly(ethyleneimine (PEI 10 K) for further stirring for 2 h at room temperature. The final product was collected through centrifugation, washing with deionized water, and freezing drying. The final product was denoted as SNPs-PEI.2-PEI.

### Example 10

### PEG Modification

15 mg of SNP-PEIs were dissolved in PEG aqueous solution (5 mg/mL) for stirring at room temperature for 12 h. The product was collected by centrifugation and washing with water and freeze drying. The final product was denoted as SNPs-PEI-PEG.

### Example 11

### In Vitro Transfection of GFP mRNA Using DMONs

In vitro transfection of GFP mRNA was evaluated in RAW264.7 cells. The commercial transfection agent, Lipofectamine 2000 was used as the positive control with the recommended dosage. Cells were seeded on the 12-well plate with a density of 1.3×10⁵ cells per well for 24 h incubation before transfection. LPS or IL-4 was added to culture medium to prime macrophage to M1 or M2 phenotype. One microgram of mRNA was mixed with 40 μg of DMONs-40-R0.2-PEI.nanoparticles s in 50 μL of 10 mM PBS solution for 30 min at room temperature, then added by droplets into each well for 48 h incubation. Cells were then harvested to determine the intracellular GFP expression through flow cytometry.

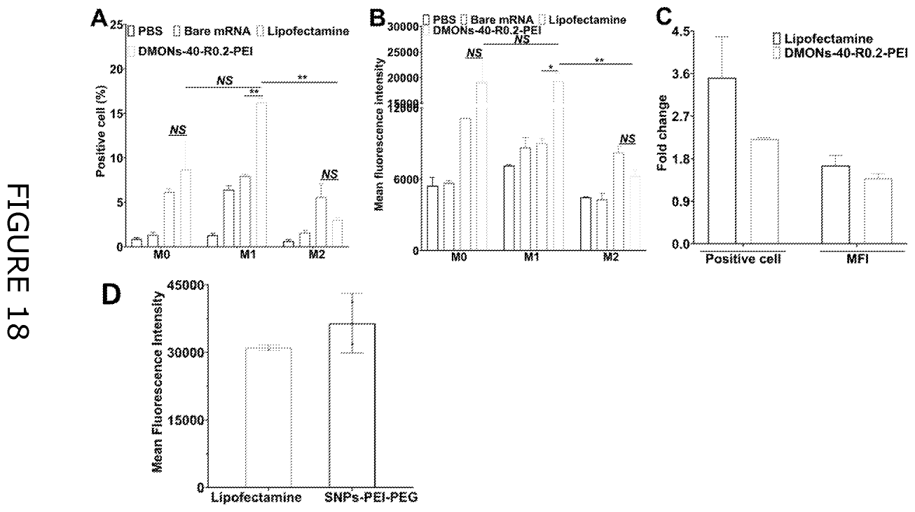

The GFP mRNA expression analyzed by FACS (FIG. 18A, B), suggested higher transfection efficiency in M1 macrophages compared to M2 and the highest transfection efficiency from DMONs-40-R0.2-PEI.compared to commercial product, Lipofectamine. After loading with chloroquine (FIG. 18C), the EGFP mRNA transfection for Lipofectamine and nanoparticles measured using flow cytometry in M2 macrophages was all increased.

The superior transfection performance of GFP mRNA delivered by SNPs-PEI-PEG was validated in RAW264.7 cells (FIG. 18D), exhibiting higher MFI compared to commercial transfection agent, Lipofectamine MessengerMax.

Alternatively, mRNA molecules in an aqueous solution can be mixed with an organic phase containing lipid molecules, such as in a microfluidic device, to form mRNA encapsulated inside liposomes.

Macrophage targeting moieties, e.g., CD47 monoclonal antibody, can be post-modified on the particle surface.

### Example 12

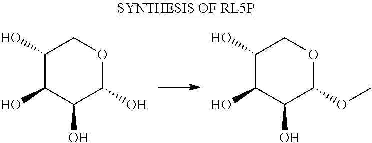

(3S,4S,5R)-2-Methoxyoxane-3,4,5-triol: A dry solution of HCl (prepared by addition of acetyl chloride (1.05 mL, 14.7 mmol)) in methanol (1 mL) was added to a solution of (3S,4S,5R)-oxane-2,3,4,5-tetrol (10.0 g, 66.6 mmol) in methanol (140 mL) and the reaction mixture was stirred at 25° C. for 16 hours. The reaction was quenched by adjusting the pH of the reaction mixture to ~7 by addition of 3 M aqueous NaOH. The solution was then evaporated to dryness and then azeotroped with toluene to give (3S,4S,5R)-2-methoxyoxane-3,4,5-triol (10 g, 60.9 mmol, 91.5% yield) as a white solid, which was used in the next step without any purification.

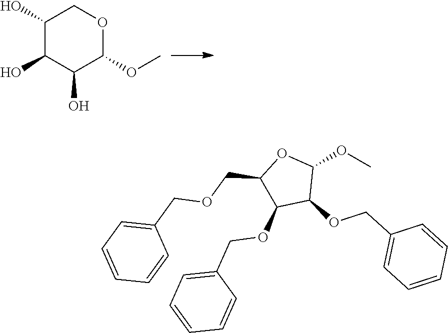 [SMILES: CO[C@H]1OC[C@@H](O)[C@H](O)[C@@H]1O ; CO[C@H]1O[C@H](COCc2ccccc2)[C@H](OCc2ccccc2)[C@@H]1OCc1ccccc1]

(3S,4S,5R)-2-Methoxyoxane-3,4,5-triol: A dry solution of HCl (prepared by addition of acetyl chloride (1.05 mL, 14.7 mmol)) in methanol (1 mL) was added to a solution of (3S,4S,5R)-oxane-2,3,4,5-tetrol (10.0 g, 66.6 mmol) in methanol (140 mL) and the reaction mixture was stirred at 25° C. for 16 hours. The reaction was quenched by adjusting the pH of the reaction mixture to ~7 by addition of 3 M aqueous NaOH. The solution was then evaporated to dryness and then azeotroped with toluene to give (3S,4S,5R)-2-methoxyoxane-3,4,5-triol (10 g, 60.9 mmol, 91.5% yield) as a white solid, which was used in the next step without any purification.

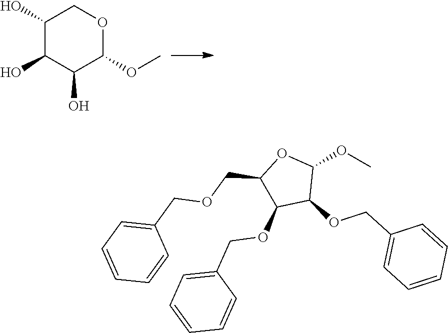 [SMILES: CO[C@H]1OC[C@@H](O)[C@H](O)[C@@H]1O ; CO[C@H]1O[C@H](COCc2ccccc2)[C@H](OCc2ccccc2)[C@@H]1OCc1ccccc1]

3S,4S,5R)-2-Methoxy-3,4-bis(phenylmethoxy)-5-(phenylmethoxymethyl)oxolane: To a stirred solution of (3S,4S,5R)-2-methoxyoxane-3,4,5-triol (10.0 g, 60.9 mmol) in N,N-dimethylformamide (30 mL) was portion wise added sodium hydride (7.31 g, 304.6 mmol) at 0° C. and the reaction mixture was stirred for 30 min at room temperature. Then TBAI (11.25 g, 30.5 mmol) and benzyl bromide (72.35 mL, 609.2 mmol) were added to the reaction dropwise at room temperature, after which the reaction was stirred at 25° C. for 2 h. After completion, the reaction was quenched with methanol (30 mL) and the reaction mixture was concentrated. The residue was taken up in ethyl acetate and washed with aqueous HCl, saturated aq. NaHCO₃ and brine. The combined organic layer was dried over Na₂SO₄, filtered and concentrated under reduced pressure. The crude mixture was purified by column chromatography using ethyl acetate in hexane to give (3S,4S,5R)-2-methoxy-3,4-bis(phenylmethoxy)-5-(phenylmethoxymethyl)oxolane (16 g, 36.8 mmol, 60.4% yield) as a sticky liquid.

LCMS: Rt=3.98 min, [MNH₄]+=452.2. Shimadzu Prominence HPLC attached with Applied Biosystems API 2000 mass spectrometer, Column-Xbridge C18 (4.6*50 mm, 5μ), Column Temperature—Ambient, Mobile Phase A: 10 mm Ammonium Acetate in water, Mobile Phase B: Acetonitrile, Flow rate: 1.20 mL/min, analysis time: 5.10 min.

### (3S,4S,5R)-3,4-Bis(phenylmethoxy)-5-(phenylmethoxymethyl)oxolan-2-ol

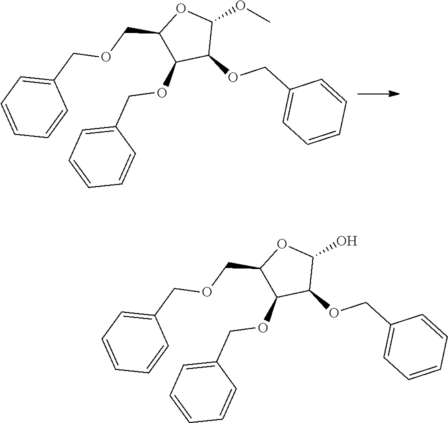 [SMILES: CO[C@H]1O[C@H](COCc2ccccc2)[C@H](OCc2ccccc2)[C@@H]1OCc1ccccc1 ; O[C@H]1O[C@H](COCc2ccccc2)[C@H](OCc2ccccc2)[C@@H]1OCc1ccccc1]

To a stirred solution of acetic acid (130 mL) and sulfuric acid (3M, 37.74 mL, 113.2 mmol) was added (3S,4S,5R)-2-methoxy-3,4-bis(phenylmethoxy)-5-(phenylmethoxymethyl)oxolane (8.2 g, 18.9 mmol). The reaction was heated at 100° C. for 1 hour, cooled to room temperature, and then neutralized with saturated aq. NaHCO₃. The mixture was extracted with dichloromethane, and the organic layer was washed with water and brine. The organic layer was then dried over sodium sulfate, filtered and evaporated to dryness. The residue was purified by flash chromatography (Ethyl acetate/hexane, 30:70) to give (3S,4S,5R)-3,4-bis(phenylmethoxy)-5-(phenylmethoxymethyl)oxolan-2-ol (5.5 g, 13.1 mmol, 69.3% yield) as off white sticky liquid.

LCMS: Rt=1.85 min, [MNH₄]+=438.0. Shimadzu Prominence HPLC attached with Applied Biosystems API 2000 mass spectrometer, Column-Xbridge C18 (4.6*50 mm, 5μ), Column Temperature—Ambient, Mobile Phase A: 5 mm Ammonium Bicarbonate in water, Mobile Phase B: Acetonitrile, Flow rate: 1.20 mL/min, analysis time: 5.10 min.

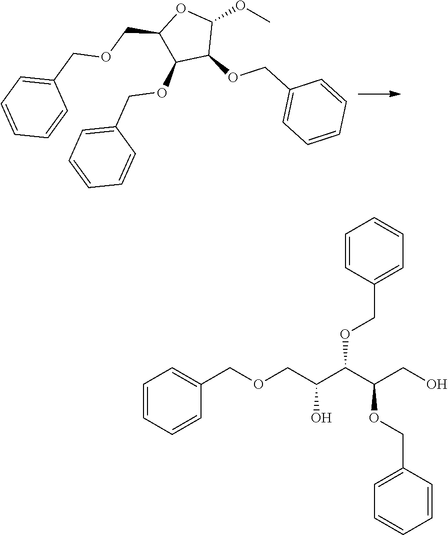 [SMILES: CO[C@H]1O[C@H](COCc2ccccc2)[C@H](OCc2ccccc2)[C@@H]1OCc1ccccc1 ; OC[C@@H](OCc1ccccc1)[C@@H](OCc1ccccc1)[C@H](O)COCc1ccccc1]

(2R,3S,4R)-2,3,5-Tris(phenylmethoxy)pentane-1,4-diol: To a stirred solution of (3S,4S,5R)-3,4-bis(phenylmethoxy)-5-(phenylmethoxymethyl)oxolan-2-ol (11.4 g, 27.1 mmol) in ethanol (70 mL) was added sodium borohydride (3.08 g, 81.3 mmol) at 0° C. and the reaction mixture was stirred at 25° C. for 5 h. After completion of the reaction, the pH of the reaction mixture was adjusted to 4-5 by the dropwise addition of acetic acid (5 mL). The resulting mixture was concentrated and then diluted with ethyl acetate. The organic layer was washed with water and brine and then dried over sodium sulfate, filtered and evaporated to dryness under reduced pressure. The residue was purified by flash chromatography to give (2R,3S,4R)-2,3,5-tris(phenylmethoxy)pentane-1,4-diol (10 g, 23.7 mmol, 87.3% yield) as off-white solid.

LCMS: Rt=3.64 min, [MH]+=423.0. Shimadzu Prominence HPLC attached with Applied Biosystems API 2000 mass spectrometer, Column-Xbridge C18 (4.6*50 mm, 5μ), Column Temperature—Ambient, Mobile Phase A: 10 mm Ammonium Acetate in water, Mobile Phase B: Acetonitrile, Flow rate: 1.20 mL/min, analysis time: 5.10 min.

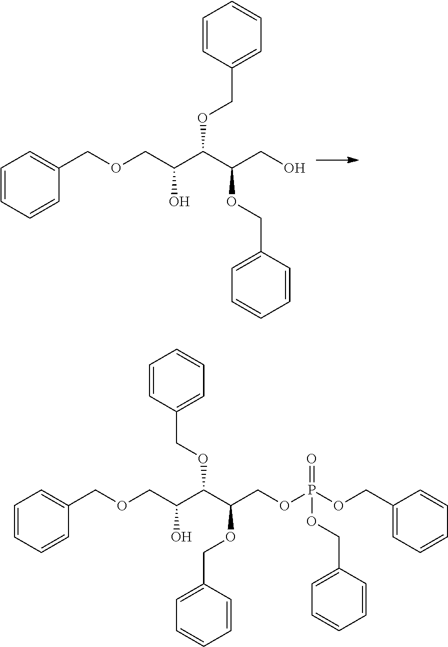 [SMILES: OC[C@@H](OCc1ccccc1)[C@@H](OCc1ccccc1)[C@H](O)COCc1ccccc1 ; O=P(OCc1ccccc1)(OCc1ccccc1)OC[C@@H](OCc1ccccc1)[C@@H](OCc1ccccc1)[C@H](O)COCc1ccccc1]

Dibenzyl [(2R,3S,4R)-4-hydroxy-2,3,5-tris(phenylmethoxy)pentyl]phosphate: To a stirred solution of (2R,3S,4R)-2,3,5-tris(phenylmethoxy)pentane-1,4-diol (5.0 g, 11.8 mmol) in tetrahydrofuran (20 mL) was added sodium hydride (662.43 mg, 16.6 mmol) (60%) portionwise at 0° C. The reaction was stirred for 10 minutes, after which chloro(phenylmethoxy)phosphoryl]oxymethylbenzene (4.92 g, 16.6 mmol) in tetrahydrofuran (8 mL) was added dropwise. The reaction was stirred at 25° C. for 30 min and then quenched by the addition of H₂O. The resulting mixture was diluted with ethyl acetate and washed with water and brine. The organic layer was dried over sodium sulfate and evaporated to dryness in vacuum. The residue was purified by flash chromatography and then further purified by Prep-HPLC to give dibenzyl [(2R,3S,4R)-4-hydroxy-2,3,5-tris(phenylmethoxy)pentyl]phosphate (1.3 g, 1.9 mmol, 16.1% yield) as yellowish white solid.

LCMS: Rt=3.45 min, [MH]+=683.2. Waters Acquity H Class UPLC attached with Waters SQD2 mass spectrometer, Column-Xbridge C18 (3*50 mm, 3.5μ), Column Temperature—40° C., Mobile Phase A: 5 mm Ammonium Acetate in water, Mobile Phase B: 5 Mm NH4oAc in ACN: Water (90:10), Flow rate: 1.20 mL/min, analysis time: 5.10 min.

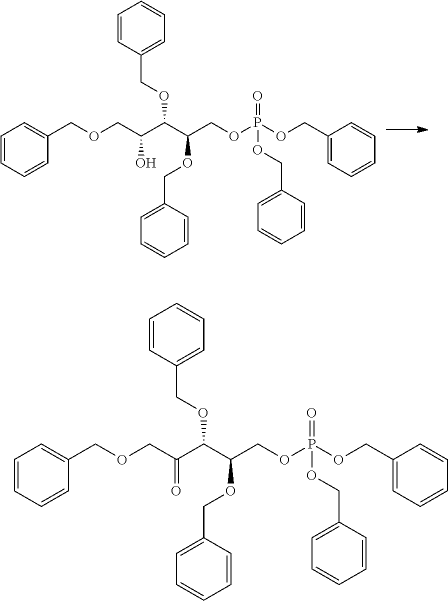 [SMILES: O=P(OCc1ccccc1)(OCc1ccccc1)OC[C@@H](OCc1ccccc1)[C@@H](OCc1ccccc1)[C@H](O)COCc1ccccc1 ; O=C(COCc1ccccc1)[C@H](OCc1ccccc1)[C@@H](COP(=O)(OCc1ccccc1)OCc1ccccc1)OCc1ccccc1]

Dibenzyl [(2R,3R)-4-oxo-2,3,5-tris(phenylmethoxy)pentyl]phosphate: Dess-Martin periodinane (1490.96 mg, 3.52 mmol) and sodium bicarbonate (984.27 mg, 11.7 mmol) were dissolved/suspended in dichloromethane (10 mL) and shaken for a few minutes. No gas evolution was observed. Reaction was then cooled to 0° C. and dibenzyl [(2R,3S,4R)-4-hydroxy-2,3,5-tris(phenylmethoxy)pentyl]phosphate (800.0 mg, 1.17 mmol) in dichloromethane (10 mL) was added. The reaction stirred and warmed to ambient temperature over 2 hours and then quenched with aqueous sodium thiosulfate/sodium bicarbonate and extracted with dichloromethane. The organic layers were washed with water and brine and then dried over magnesium sulfate and evaporated to dryness in vacuum. The crude residue was purified by flash column chromatography to give dibenzyl [(2R,3R)-4-oxo-2,3,5-tris(phenylmethoxy)pentyl]phosphate (660 mg, 0.921 mmol, 78.6% yield).

¹H NMR (400 MHz, Chloroform-d): δ 7.59-7.06 (m, 22H), 5.08-4.96 (m, 4H), 4.64-4.57 (m, 1H), 4.55-4.39 (m, 5H), 4.32-4.23 (m, 2H), 4.21-4.10 (m, 3H), 4.00-3.94 (m, 1H).

LCMS: Rt 3.58 mins, [MH]+=681 (100) Column: XBridge BEH C18, 2.1×50 mm, 2.5 micron, Column temperature: 40° C., Mobile Phase A: H2O+0.1% Formic Acid, Mobile Phase B: MeCN, Mobile phase gradient details: T=0 min (95% A, 5% B); T=0.3 min (95% A, 5% B); gradient to T=3 min (5% A, 95% B); end of run at T=4 min (5% A, 95% B), Flow rate: 0.5 mL/min, analysis time 5.5 min.

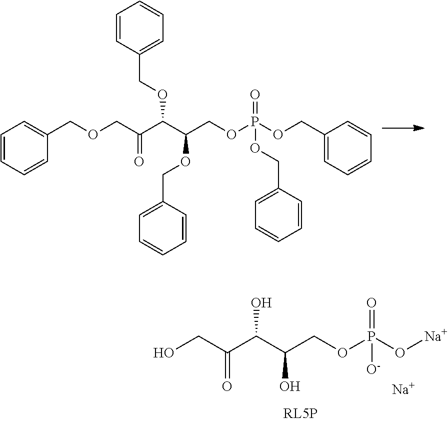

Disodium [(2R,3R)-2,3,5-trihydroxy-4-oxopentyl]phosphate (RL5P): Dibenzyl [(2R,3R)-4-oxo-2,3,5-tris(phenylmethoxy)pentyl]phosphate (575.0 mg, 0.840 mmol) and palladium 10% on activated carbon (wetted with ca. 53% water) (179.78 mg, 0.170 mmol) were dissolved/suspended in ethanol (20 mL), degassed under a nitrogen atmosphere, degassed under a hydrogen atmosphere, and then stirred at ambient under a hydrogen atmosphere for 4 h. The reaction was filtered, and the Pd/C residue was washed with water, and the solution was neutralized to pH7 with aqueous sodium hydroxide (0.2 M) (7.6 mL, 1.52 mmol). The ethanol was evaporated in vacuo at ambient temperature, and then the water was removed on the freeze drier to give disodium [(2R,3R)-2,3,5-trihydroxy-4-oxopentyl]phosphate (RL5P) (230 mg, 0.797 mmol, 94.4% yield).

¹H NMR (400 MHz, Deuterium Oxide): δ 4.61 (s, 1H), 4.57 (d, J=19.4 Hz, 1H), 4.43 (d, J=6.0 Hz, 1H), 4.01 (q, J=4.9 Hz, 1H), 3.88 (ddq, J=16.6, 11.2, 6.4, 5.4 Hz, 2H).

LCMS: Rt 0.51 mins, [MH]+=231, 461 (2M+H), 483 (2M+Na), 253 (M+Na, 100), [M−H]−=229, 459 (100).

Column: XBridge BEH C18, 2.1×50 mm, 2.5 micron, Column temperature: 40° C., Mobile Phase A: H2O+0.1% Formic Acid, Mobile Phase B: MeCN, Mobile phase gradient details: T=0 min (95% A, 5% B); T=0.3 min (95% A, 5% B); gradient to T=3 min (5% A, 95% B); end of run at T=4 min (5% A, 95% B), Flow rate: 0.5 mL/min, analysis time 5.5 min.

### Example 13

### Encapsulation of RL5P

RL5P molecules can be encapsulated inside liposomes during synthesis via microfluidic methods. Briefly, stocks of lipids will be dissolved in ethanol and mixed in the appropriate molar ratios to obtain a lipid concentration of 12.5 mM. mRNA and RL5P will be dissolved in RNase free 50 mM citrate buffer pH 3.0 to obtain a mRNA:lipid weight ratio of 10:1. Empty LNPs will be also prepared using 50 mM citrate buffer as the aqueous phase. The aqueous and ethanol solutions will be mixed in a 3:1 volume ratio using a microfluidic device NanoAssemblr, with a mixing rate of 12 mL per minute. The RL5P encapsulated formulation will be collected via dialysis against water overnight using dialysis cassettes purchased from Thermo Scientific with a molecular cutoff of 10 K. Final product will be concentrated using Amicon ultracentrifugation filters and freeze-drying.

### Example 14

### 6PGD Activation by Co-Delivery of HDAC7

6PGD activity can be enhanced by co-delivering HDAC7. HEK293 cells were plated in 12-well plates and incubated overnight at 37° C. The Lipofectamine 2000 (Invitrogen) transfection reagent was prepared in OptiMEM to a final concentration of 1 μg DNA per μL and incubated at room temperature for 5 min. Plasmid DNA was diluted in OptiMEM (Gibco) and then mixed with Lipofectamine 2000, after which the transfection mixture was incubated at room temperature for 30 min. Prior to transfection, cell culture media was replaced with OptiMEM, after which the transfection mixture was added into cells. Cells were then incubated at 37° C. for 6-8 h before being supplemented with fresh OptiMEM. 24 h post-transfection, culture supernatants were discarded, and cells were briefly washed with DPBS and lysed for different analyses.

Mouse HDAC7 (the unspliced isoform lacking the first 22 amino acids), an enzyme-dead (ED) mutant HDAC7 carrying an H-to-A mutation (Das Gupta et al., 2020, Cell Rep. 30:2712-2728.e2718), N-terminal HDAC7 (N-HDAC7, amino acid 23-504, comprising a PHA03247 super family motif corresponding to the herpes simplex virus-1 UL36 large tegument protein, which is known to comprise deubiquitinating activity) and C-terminal HDAC7 (C-HDAC7, amino acid 498-938) were cloned in pEF6/V5-His-TOPO vectors. Mouse 6PGD was cloned in a pCMV expression vector (MG53165-CF, Sino Biologicals). HEK293 cells were transiently transfected with expression constructs encoding 6PGD and HDAC7 at a molar ratio of 1:1. 24 h after transfection, cells were lysed and lysates were subjected to 6PGD activity assays using an in vitro assay (Abcam), as per the manufacturer's instructions. The enzymatic activity was determined by measuring absorbance at 460 nm in kinetic mode using a plate reader (Infinite M Plex, Tecan).

The 6PGD amino acid sequence used for HEK293 transfection is the sequence set forth in SEQ ID NO:3, and the corresponding coding sequence for 6PGD is set forth in SEQ ID NO:7.

The HDAC7 amino acid sequence used for HEK293 transfection is the sequence set forth in SEQ ID NO:13, and the corresponding coding sequence for that HDAC7 amino acid sequence is set forth in SEQ ID NO:20.

The HDAC7 ED amino acid sequence used for HEK293 transfection is the sequence set forth in SEQ ID NO:14, and the corresponding coding sequence for that HDAC7 amino acid sequence is set forth in SEQ ID NO:21.

The N-terminal HDAC7 amino acid sequence used for HEK293 transfection is the sequence set forth in SEQ ID NO:15, and the corresponding coding sequence for that HDAC7 amino acid sequence is set forth in SEQ ID NO:22.

The C-terminal HDAC7 amino acid sequence used for HEK293 transfection is:

[SEQ ID NO: 23]
MVLNSSETPATGLVYDSVMLKHQCSCGDNSKHPEHAGRIQSIWSRLQER
GLRSQCECLRGRKASLEELQSVHSERHVLLYGTNPLSRLKLDNGKLTGL
LAQRTFVMLPCGGVGVDTDTIWNELHSSNAARWAAGSVTDLAFKVASRE
LKNGFAVVRPPGHHADHSTAMGFCFFNSVAIACRQLQQHGKASKILIVD
WDVHHGNGTQQTFYQDPSVLYISLHRHDDGNFFPGSGAVDEVGTGSGEG
FNVNVAWAGGLDPPMGDPEYLAAFRIVVMPIAREFAPDLVLVSAGFDAA
EGHPAPLGGYHVSAKCFGYMTQQLMNLAGGAVVLALEGGHDLTAICDAS
EACVAALLGNKVDPLSEESWKQKPNLSAIRSLEAVVRVHRKYWGCMQRL
ASCPDSWLPRVPGADAEVEAVTALASLSVGILAEDRPSERLVEEEEPMN
L, wherein the motif corresponding to the HDAC7
enzymaticdomain is underlined.

The corresponding coding sequence for C-terminal HDAC7 is:

[SEQ ID NO: 24]
atggtcctcaacagctcagagacacctgctacagggctggtctatgactcggtgatgctgaaacaccaatgttcct
gtggagacaacagcaagcatcccgagcatgcaggccgcatccagagcatctggtcccggctgcaggaacggggtctccgcagccagtgtg
agtgtctccgaggccgaaaggcttccctagaggagctgcagtcagtccactctgaacggcacgtgctcctctacggcacgaacccactcag
ccgcctcaaactggataacgggaagcttacagggctcctggcacagcggacgtttgtgatgctaccctgtggcggggttggggtcgatact
gacaccatctggaacgagctgcattcctccaatgcagcccgctgggctgcgggcagcgtcaccgaccttgccttcaaagtagcttcccgag
agctgaagaacggctttgctgtggtgcgacccccgggacaccatgcagatcattctacagccatgggcttctgcttcttcaactccgtggc
catcgcctgccgacagctacagcaacacggcaaagccagcaagatcctcattgttgactgggatgttcaccatggcaacggcacacagcag
actttctaccaggaccccagtgtgctctacatttcccttcatcgtcatgacgacggcaacttcttcccaggcagtggggccgtggatgagg
tgggaactggcagtggcgagggcttcaatgtcaacgtggcttgggctgggggcttggatccacccatgggggatcctgagtacctggctgc
tttcaggatagtggtgatgcccattgcccgagagtttgctccagacctggtcctggtgtctgctgggtttgatgctgcggagggtcaccca
gccccactgggtggctaccatgtttctgccaaatgttttgggtacatgacgcagcagttgatgaacttggcaggaggcgccgtggtgttgg
ccttagagggtggacatgacctcacggccatctgtgatgcctcggaggcctgtgtagctgctcttctgggcaacaaggtggaccccctttc
agaagaaagctggaaacagaaacccaacctcagtgccatccgctcgctggaagctgtggtcagggtgcacaggaaatactggggctgcatg
cagcgcttggcctcctgtccagactcctggctacccagagtgccgggagctgatgcagaagtggaagccgtgaccgcgctggcatcccttt
ctgtgggcatcctggctgaagacaggccctcggagcggctggtggaagaggaagaacccatgaacctc.

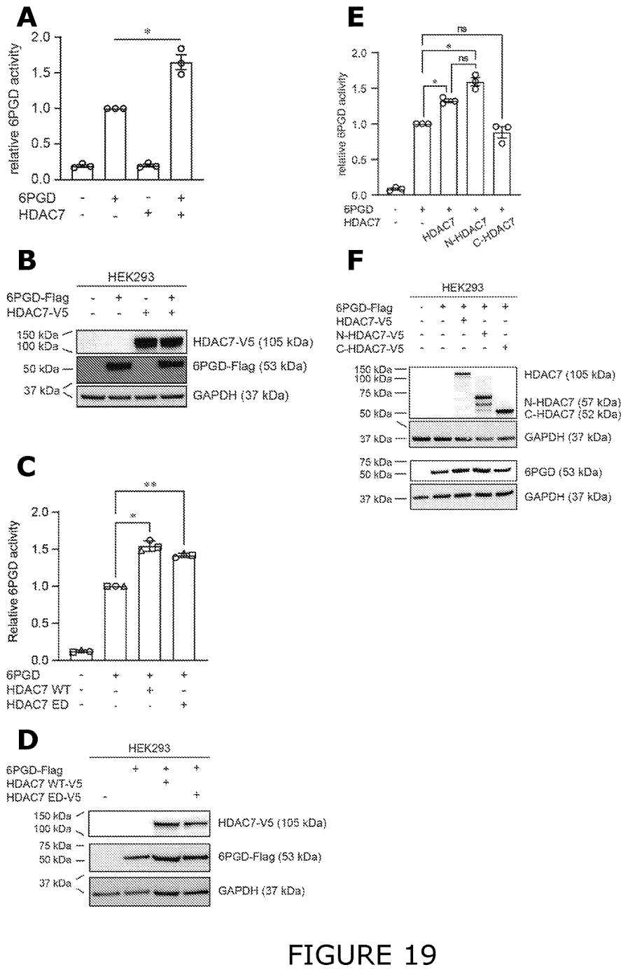

HDAC7 significantly increases 6PGD activity in HEK293 cells by comparison to cells transfected with an expression construct for 6PGD alone (FIG. 19A). HDAC7 and 6PGD expression levels were confirmed by western blot analysis (FIG. 19B).

In comparison to cells transfected with 6PGD alone, both WT and ED HDAC7 significantly increased the enzymatic activity of 6PGD to a similar level. This indicates HDAC7-mediated 6PGD activity is independent of the deacetylase activity of HDAC7 (FIG. 19C). HDAC7 and 6PGD expression levels were confirmed by western blot analysis (FIG. 19D).

The N-terminal domain of HDAC7 increased the enzymatic activity of 6PGD to similar levels to that of the WT HDAC7. The C-terminal deacetylase domain of HDAC7 does not activate 6PGD (FIG. 19E). HDAC7 and 6PGD expression levels were confirmed by western blot analysis (FIG. 19F).

The disclosure of every patent, patent application, and publication cited herein is hereby incorporated herein by reference in its entirety.

The citation of any reference herein should not be construed as an admission that such reference is available as “Prior Art” to the instant application.

Throughout the specification the aim has been to describe the preferred embodiments of the disclosure without limiting the disclosure to any one embodiment or specific collection of features. Those of skill in the art will therefore appreciate that, in light of the instant disclosure, various modifications and changes can be made in the particular embodiments exemplified without departing from the scope of the present disclosure. All such modifications and changes are intended to be included within the scope of the appended claims.

1. A method for inhibiting or reducing pro-inflammatory activity of an antigen-presenting cell, the method comprising contacting the antigen-presenting cell with a therapeutic agent, the therapeutic agent comprising D-ribulose-5-phosphate (RL5P), and/or a RL5P-producing agent, wherein, when present, the RL5P-producing agent is 6-phosphogluconate dehydrogenase (6PGD), or a nucleic acid molecule from which 6PGD is producible.

2. (canceled)

3. The method of claim 1, wherein the antigen-presenting cell is selected from a macrophage, a dendritic cell and a B-cell.

4. The method of claim 3, wherein the antigen-presenting cell is a macrophage.

5. The method of claim 1, wherein the pro-inflammatory activity comprises the production and/or release of a pro-inflammatory mediator.

6. The method of claim 5, wherein the pro-inflammatory mediator is a pro-inflammatory cytokine selected from the group consisting of IL-1β and TNF.

7.-8. (canceled)

9. The method of claim 1, wherein the therapeutic agent comprises a nanoparticle or microparticle capable of being taken up by the antigen-presenting cell.

10. The method of claim 1, wherein the therapeutic agent comprises a nucleic acid molecule from which 6PGD is producible, wherein the nucleic acid molecule comprises a 6PGD coding sequence.

11. The method of claim 1, further comprising contacting the antigen-presenting cell with an HDAC7 polypeptide-producing agent.

12. A therapeutic agent comprising D-ribulose-5-phosphate (RL5P) and/or a RL5P-producing agent wherein, when present, the RL5P-producing agent is 6-phosphogluconate dehydrogenase (6PGD), or a nucleic acid molecule from which 6PGD is producible.

13. (canceled)

14. The therapeutic agent of claim 12, wherein the therapeutic agent comprises a nanoparticle or microparticle that is capable of being taken up by an antigen-presenting cell.

15. The therapeutic agent of claim 12, wherein the therapeutic agent comprises a nucleic acid molecule from which 6PGD is producible, wherein the nucleic acid molecule comprises, consists or consists essentially of a 6PGD coding sequence.

16. The therapeutic agent of claim 15, wherein the nucleic acid molecule comprises mRNA.

17. The therapeutic agent of claim 15, wherein the nucleic acid molecule comprises DNA.

18. The therapeutic agent of claim 15, wherein the coding sequence is operably connected to a regulatory element that is operable in an antigen-presenting cell.

19. The therapeutic agent of claim 12, wherein the therapeutic agent targets an antigen-presenting cell.

20.-21. (canceled)

22. A method for treating, preventing, inhibiting or reducing, or slowing the progression of, inflammation in a subject, the method comprising administering to the subject an effective amount of the therapeutic agent of claim 12.

23. A method for treating an acute inflammatory condition in a subject, the method comprising administering to the subject an effective amount of the therapeutic agent of claim 12.

24. A method for treating a chronic inflammatory condition in a subject, the method comprising administering to the subject an effective amount of the therapeutic agent of claim 12.

25. (canceled)


SEQUENCE LISTING (24 sequences)

SEQ ID NO: 1 - protein, 483 aa, Homo sapiens
MAQADIALIGLAVMGQNLILNMNDHGFVVCAFNRTVSKVDDFLANEAKGTKVVGAQSLKEMVSKLKKPRRIILLVKAGQAVDDFIEKLVPLLDTGDIIIDGGNSEYRDTTRRCRDLKAKGILFVGSGVSGGEEGARYGPSLMPGGNKEAWPHIKTIFQGIAAKVGTGEPCCDWVGDEGAGHFVKMVHNGIEYGDMQLICEAYHLMKDVLGMAQDEMAQAFEDWNKTELDSFLIEITANILKFQDTDGKHLLPKIRDSAGQKGTGKWTAISALEYGVPVTLIGEAVFARCLSSLKDERIQASKKLKGPQKFQFDGDKKSFLEDIRKALYASKIISYAQGFMLLRQAATEFGWTLNYGGIALMWRGGCIIRSVFLGKIKDAFDRNPELQNLLLDDFFKSAVENCQDSWRRAVSTGVQAGIPMPCFTTALSFYDGYRHEMLPASLIQAQRDYFGAHTYELLAKPGQFIHTNWTGHGGTVSSSSYNA

SEQ ID NO: 2 - protein, 460 aa, Homo sapiens
MGQNLILNMNDHGFVVCAFNRTVSKVDDFLANEAKGTKVVGAQSLKEMVSKLKKPRRIILLVKAGQAVDDFIEKLVPLLDTGDIIIDGGNSEYRDTTRRCRDLKAKGILFVGSGVSGGEEGARYGPSLMPGGNKEAWPHIKTIFQGIAAKVGTGEPCCDWVGDEGAGHFVKMVHNGIEYGDMQLICEAYHLMKDVLGMAQDEMAQAFEDWNKTELDSFLIEITANILKFQDTDGKHLLPKIRDSAGQKGTGKWTAISALEYGVPVTLIGEAVFARCLSSLKDERIQASKKLKGPQKFQFDGDKKSFLEDIRKALYASKIISYAQGFMLLRQAATEFGWTLNYGGIALMWRGGCIIRSVFLGKIKDAFDRNPELQNLLLDDFFKSAVENCQDSWRRAVSTGVQAGIPMPCFTTALSFYDGYRHEMLPASLIQAQRDYFGAHTYELLAKPGQFIHTNWTGHG

SEQ ID NO: 3 - protein, 483 aa, Mus musculus
MAQADIALIGLAVMGQNLILNMNDHGFVVCAFNRTVSKVDDFLANEAKGTKVVGAQSLKDMVSKLKKPRRVILLVKAGQAVDDFIEKLVPLLDTGDIIIDGGNSEYRDTTRRCRDLKAKGILFVGSGVSGGEEGARYGPSLMPGGNKEAWPHIKAIFQAIAAKVGTGEPCCDWVGDEGAGHFVKMVHNGIEYGDMQLICEAYHLMKDVLGMRHEEMAQAFEEWNKTELDSFLIEITANILKYRDTDGKELLPKIRDSAGQKGTGKWTAISALEYGMPVTLIGEAVFARCLSSLKEERVQASQKLKGPKVVQLEGSKKSFLEDIRKALYASKIISYAQGFMLLRQAATEFGWTLNYGGIALMWRGGCIIRSVFLGKIKDAFERNPELQNLLLDDFFKSAVDNCQDSWRRVISTGVQAGIPMPCFTTALSFYDGYRHEMLPANLIQAQRDYFGAHTYELLTKPGEFIHTNWTGHGGSVSSSSYNA

SEQ ID NO: 4 - protein, 457 aa, Mus musculus
MGQNLILNMNDHGFVVCAFNRTVSKVDDFLANEAKGTKVVGAQSLKDMVSKLKKPRRVILLVKAGQAVDDFIEKLVPLLDTGDIIIDGGNSEYRDTTRRCRDLKAKGILFVGSGVSGGEEGARYGPSLMPGGNKEAWPHIKAIFQAIAAKVGTGEPCCDWVGDEGAGHFVKMVHNGIEYGDMQLICEAYHLMKDVLGMRHEEMAQAFEEWNKTELDSFLIEITANILKYRDTDGKELLPKIRDSAGQKGTGKWTAISALEYGMPVTLIGEAVFARCLSSLKEERVQASQKLKGPKVVQLEGSKKSFLEDIRKALYASKIISYAQGFMLLRQAATEFGWTLNYGGIALMWRGGCIIRSVFLGKIKDAFERNPELQNLLLDDFFKSAVDNCQDSWRRVISTGVQAGIPMPCFTTALSFYDGYRHEMLPANLIQAQRDYFGAHTYELLTKPGEFIHTNWT

SEQ ID NO: 5 - DNA, 1449 bp, Homo sapiens
  source 1..1449: mol_type = other DNA
atggcccaagctgacatcgcgctgatcggattggccgtcatgggccagaacttaattctgaacatgaatgaccacggctttgtggtctgtgcttttaataggactgtctccaaagttgatgatttcttggccaatgaggcaaagggaaccaaagtggtgggtgcccagtccctgaaagagatggtctccaagctgaagaagccccggcggatcatcctcctggtgaaggctgggcaagctgtggatgatttcatcgagaaattggtaccattgttggatactggtgacatcatcattgacggaggaaattctgaatatagggacaccacaagacggtgccgagacctcaaggccaagggaattttatttgtggggagcggagtcagtggtggagaggaaggggcccggtatggcccatcgctcatgccaggagggaacaaagaagcgtggccccacatcaagaccatcttccaaggcattgctgcaaaagtgggaactggagaaccctgctgtgactgggtgggagatgagggagcaggccacttcgtgaagatggtgcacaacgggatagagtatggggacatgcagctgatctgtgaggcataccacctgatgaaagacgtgctgggcatggcgcaggacgagatggcccaggcctttgaggattggaataagacagagctagactcattcctgattgaaatcacagccaatattctcaagttccaagacaccgatggcaaacacctgctgccaaagatcagggacagcgcggggcagaagggcacagggaagtggaccgccatctccgccctggaatacggcgtacccgtcaccctcattggagaagctgtctttgctcggtgcttatcatctctgaaggatgagagaattcaagctagcaaaaagctgaagggtccccagaagttccagtttgatggtgataagaaatcattcctggaggacattcggaaggcactctacgcttccaagatcatctcttacgctcaaggctttatgctgctaaggcaggcagccaccgagtttggctggactctcaattatggtggcatcgccctgatgtggagagggggctgcatcattagaagtgtattcctaggaaagataaaggatgcatttgatcgaaacccggaacttcagaacctcctactggacgacttctttaagtcagctgttgaaaactgccaggactcctggcggcgggcagtcagcactggggtccaggctggcattcccatgccctgttttaccactgccctctccttctatgacgggtacagacatgagatgcttccagccagcctcatccaggctcagcgggattacttcggggctcacacctatgaactcttggccaaaccagggcagtttatccacaccaactggacaggccatggtggcaccgtgtcatcctcgtcatacaatgcc

SEQ ID NO: 6 - DNA, 1380 bp, Homo sapiens
  source 1..1380: mol_type = other DNA
atgggccagaacttaattctgaacatgaatgaccacggctttgtggtctgtgcttttaataggactgtctccaaagttgatgatttcttggccaatgaggcaaagggaaccaaagtggtgggtgcccagtccctgaaagagatggtctccaagctgaagaagccccggcggatcatcctcctggtgaaggctgggcaagctgtggatgatttcatcgagaaattggtaccattgttggatactggtgacatcatcattgacggaggaaattctgaatatagggacaccacaagacggtgccgagacctcaaggccaagggaattttatttgtggggagcggagtcagtggtggagaggaaggggcccggtatggcccatcgctcatgccaggagggaacaaagaagcgtggccccacatcaagaccatcttccaaggcattgctgcaaaagtgggaactggagaaccctgctgtgactgggtgggagatgagggagcaggccacttcgtgaagatggtgcacaacgggatagagtatggggacatgcagctgatctgtgaggcataccacctgatgaaagacgtgctgggcatggcgcaggacgagatggcccaggcctttgaggattggaataagacagagctagactcattcctgattgaaatcacagccaatattctcaagttccaagacaccgatggcaaacacctgctgccaaagatcagggacagcgcggggcagaagggcacagggaagtggaccgccatctccgccctggaatacggcgtacccgtcaccctcattggagaagctgtctttgctcggtgcttatcatctctgaaggatgagagaattcaagctagcaaaaagctgaagggtccccagaagttccagtttgatggtgataagaaatcattcctggaggacattcggaaggcactctacgcttccaagatcatctcttacgctcaaggctttatgctgctaaggcaggcagccaccgagtttggctggactctcaattatggtggcatcgccctgatgtggagagggggctgcatcattagaagtgtattcctaggaaagataaaggatgcatttgatcgaaacccggaacttcagaacctcctactggacgacttctttaagtcagctgttgaaaactgccaggactcctggcggcgggcagtcagcactggggtccaggctggcattcccatgccctgttttaccactgccctctccttctatgacgggtacagacatgagatgcttccagccagcctcatccaggctcagcgggattacttcggggctcacacctatgaactcttggccaaaccagggcagtttatccacaccaactggacaggccatggt

SEQ ID NO: 7 - DNA, 1449 bp, Mus musculus
  source 1..1449: mol_type = other DNA
atggcccaagctgacattgcactgatcggactggctgtcatgggccagaacttaattttgaacatgaatgatcatggatttgtggtctgtgctttcaataggacagtctccaaagtcgatgacttcttggccaacgaggcgaagggcaccaaggtggttggtgcacagtccttaaaggacatggtctccaaactaaagaagccccgccgggtcatcctgcttgtgaaggccgggcaagccgtggatgatttcatcgagaaattagtacccttgttggacacgggtgacatcatcatcgatggaggaaattctgaataccgggacacgacaagaagatgccgggacctcaaggccaagggcatcttgtttgtggggagcggagtcagtggtggtgaggaaggggctcggtacgggccgtcactcatgccaggagggaacaaagaggcttggccccacatcaaggcgatcttccaagccatcgctgcaaaagtgggaaccggagaaccctgctgtgactgggtgggagatgagggggcggggcactttgtgaagatggtgcacaacgggatagagtatggagacatgcagctcatctgtgaggcttaccatttgatgaaggatgttctgggcatgcggcatgaggagatggctcaggcatttgaagaatggaacaagacagagctggactcattcctgattgaaatcactgctaacattctcaagtaccgggacactgacggcaaagagctgttgccaaagatccgggacagtgctgggcagaagggcactgggaagtggaccgccatctcggcgctggagtacggcatgcccgtcaccctcattggagaagctgtctttgctcggtgcttgtcctctctgaaggaggagcgggttcaggccagccaaaagctgaagggtcctaaggtggtccagctggaaggcagtaagaagtcattcctggaggacatccgtaaggccctctatgcttccaagatcatctcctacgcccaaggctttatgctgctcagacaggcagccactgagtttggctggaccctcaattatggcggcattgccctgatgtggagagggggctgcatcatccgaagtgtgttcctgggaaaaattaaagatgcatttgagcgaaacccagaacttcagaacctactgctagatgacttctttaagtcagcagttgacaactgccaggactcctggcggcgggtgatcagcactggggtgcaagcaggcattcccatgccctgcttcactactgccctctccttctatgatgggtacagacacgagatgctgccagcaaacctcatccaggctcaacgggattactttggggctcacacctatgaactcttaaccaaaccgggagaatttatccacaccaactggacgggccacgggggcagtgtgtcatcctcttcatacaatgcc

SEQ ID NO: 8 - DNA, 1371 bp, Mus musculus
  source 1..1371: mol_type = other DNA
atgggccagaacttaattttgaacatgaatgatcatggatttgtggtctgtgctttcaataggacagtctccaaagtcgatgacttcttggccaacgaggcgaagggcaccaaggtggttggtgcacagtccttaaaggacatggtctccaaactaaagaagccccgccgggtcatcctgcttgtgaaggccgggcaagccgtggatgatttcatcgagaaattagtacccttgttggacacgggtgacatcatcatcgatggaggaaattctgaataccgggacacgacaagaagatgccgggacctcaaggccaagggcatcttgtttgtggggagcggagtcagtggtggtgaggaaggggctcggtacgggccgtcactcatgccaggagggaacaaagaggcttggccccacatcaaggcgatcttccaagccatcgctgcaaaagtgggaaccggagaaccctgctgtgactgggtgggagatgagggggcggggcactttgtgaagatggtgcacaacgggatagagtatggagacatgcagctcatctgtgaggcttaccatttgatgaaggatgttctgggcatgcggcatgaggagatggctcaggcatttgaagaatggaacaagacagagctggactcattcctgattgaaatcactgctaacattctcaagtaccgggacactgacggcaaagagctgttgccaaagatccgggacagtgctgggcagaagggcactgggaagtggaccgccatctcggcgctggagtacggcatgcccgtcaccctcattggagaagctgtctttgctcggtgcttgtcctctctgaaggaggagcgggttcaggccagccaaaagctgaagggtcctaaggtggtccagctggaaggcagtaagaagtcattcctggaggacatccgtaaggccctctatgcttccaagatcatctcctacgcccaaggctttatgctgctcagacaggcagccactgagtttggctggaccctcaattatggcggcattgccctgatgtggagagggggctgcatcatccgaagtgtgttcctgggaaaaattaaagatgcatttgagcgaaacccagaacttcagaacctactgctagatgacttctttaagtcagcagttgacaactgccaggactcctggcggcgggtgatcagcactggggtgcaagcaggcattcccatgccctgcttcactactgccctctccttctatgatgggtacagacacgagatgctgccagcaaacctcatccaggctcaacgggattactttggggctcacacctatgaactcttaaccaaaccgggagaatttatccacaccaactggacg

SEQ ID NO: 9 - protein, 991 aa, Homo sapiens
MHSPGADGTQVSPGAHYCSPTGAGCPRPCADTPGPQPQPMDLRVGQRPPVEPPPEPTLLALQRPQRLHHHLFLAGLQQQRSVEPMRLSMDTPMPELQVGPQEQELRQLLHKDKSKRSAVASSVVKQKLAEVILKKQQAALERTVHPNSPGIPYRTLEPLETEGATRSMLSSFLPPVPSLPSDPPEHFPLRKTVSEPNLKLRYKPKKSLERRKNPLLRKESAPPSLRRRPAETLGDSSPSSSSTPASGCSSPNDSEHGPNPILGSEALLGQRLRLQETSVAPFALPTVSLLPAITLGLPAPARADSDRRTHPTLGPRGPILGSPHTPLFLPHGLEPEAGGTLPSRLQPILLLDPSGSHAPLLTVPGLGPLPFHFAQSLMTTERLSGSGLHWPLSRTRSEPLPPSATAPPPPGPMQPRLEQLKTHVQVIKRSAKPSEKPRLRQIPSAEDLETDGGGPGQVVDDGLEHRELGHGQPEARGPAPLQQHPQVLLWEQQRLAGRLPRGSTGDTVLLPLAQGGHRPLSRAQSSPAAPASLSAPEPASQARVLSSSETPARTLPFTTGLIYDSVMLKHQCSCGDNSRHPEHAGRIQSIWSRLQERGLRSQCECLRGRKASLEELQSVHSERHVLLYGTNPLSRLKLDNGKLAGLLAQRMFVMLPCGGVGVDTDTIWNELHSSNAARWAAGSVTDLAFKVASRELKNGFAVVRPPGHHADHSTAMGFCFFNSVAIACRQLQQQSKASKILIVDWDVHHGNGTQQTFYQDPSVLYISLHRHDDGNFFPGSGAVDEVGAGSGEGFNVNVAWAGGLDPPMGDPEYLAAFRIVVMPIAREFSPDLVLVSAGFDAAEGHPAPLGGYHVSAKCFGYMTQQLMNLAGGAVVLALEGGHDLTAICDASEACVAALLGNRVDPLSEEGWKQKPNLNAIRSLEAVIRVHSKYWGCMQRLASCPDSWVPRVPGADKEEVEAVTALASLSVGILAEDRPSEQLVEEEEPMNL

SEQ ID NO: 10 - protein, 952 aa, Homo sapiens
MDLRVGQRPPVEPPPEPTLLALQRPQRLHHHLFLAGLQQQRSVEPMRLSMDTPMPELQVGPQEQELRQLLHKDKSKRSAVASSVVKQKLAEVILKKQQAALERTVHPNSPGIPYRTLEPLETEGATRSMLSSFLPPVPSLPSDPPEHFPLRKTVSEPNLKLRYKPKKSLERRKNPLLRKESAPPSLRRRPAETLGDSSPSSSSTPASGCSSPNDSEHGPNPILGSEALLGQRLRLQETSVAPFALPTVSLLPAITLGLPAPARADSDRRTHPTLGPRGPILGSPHTPLFLPHGLEPEAGGTLPSRLQPILLLDPSGSHAPLLTVPGLGPLPFHFAQSLMTTERLSGSGLHWPLSRTRSEPLPPSATAPPPPGPMQPRLEQLKTHVQVIKRSAKPSEKPRLRQIPSAEDLETDGGGPGQVVDDGLEHRELGHGQPEARGPAPLQQHPQVLLWEQQRLAGRLPRGSTGDTVLLPLAQGGHRPLSRAQSSPAAPASLSAPEPASQARVLSSSETPARTLPFTTGLIYDSVMLKHQCSCGDNSRHPEHAGRIQSIWSRLQERGLRSQCECLRGRKASLEELQSVHSERHVLLYGTNPLSRLKLDNGKLAGLLAQRMFVMLPCGGVGVDTDTIWNELHSSNAARWAAGSVTDLAFKVASRELKNGFAVVRPPGHHADHSTAMGFCFFNSVAIACRQLQQQSKASKILIVDWDVHHGNGTQQTFYQDPSVLYISLHRHDDGNFFPGSGAVDEVGAGSGEGFNVNVAWAGGLDPPMGDPEYLAAFRIVVMPIAREFSPDLVLVSAGFDAAEGHPAPLGGYHVSAKCFGYMTQQLMNLAGGAVVLALEGGHDLTAICDASEACVAALLGNRVDPLSEEGWKQKPNLNAIRSLEAVIRVHSKYWGCMQRLASCPDSWVPRVPGADKEEVEAVTALASLSVGILAEDRPSEQLVEEEEPMNL

SEQ ID NO: 11 - protein, 511 aa, Homo sapiens
MDLRVGQRPPVEPPPEPTLLALQRPQRLHHHLFLAGLQQQRSVEPMRLSMDTPMPELQVGPQEQELRQLLHKDKSKRSAVASSVVKQKLAEVILKKQQAALERTVHPNSPGIPYRTLEPLETEGATRSMLSSFLPPVPSLPSDPPEHFPLRKTVSEPNLKLRYKPKKSLERRKNPLLRKESAPPSLRRRPAETLGDSSPSSSSTPASGCSSPNDSEHGPNPILGSEALLGQRLRLQETSVAPFALPTVSLLPAITLGLPAPARADSDRRTHPTLGPRGPILGSPHTPLFLPHGLEPEAGGTLPSRLQPILLLDPSGSHAPLLTVPGLGPLPFHFAQSLMTTERLSGSGLHWPLSRTRSEPLPPSATAPPPPGPMQPRLEQLKTHVQVIKRSAKPSEKPRLRQIPSAEDLETDGGGPGQVVDDGLEHRELGHGQPEARGPAPLQQHPQVLLWEQQRLAGRLPRGSTGDTVLLPLAQGGHRPLSRAQSSPAAPASLSAPEPASQARVLSSSET

SEQ ID NO: 12 - protein, 938 aa, Mus musculus
MHSPGAGCPALQPDTPGSQPQPMDLRVGQRPTVEPPPEPALLTLQHPQRLHRHLFLAGLHQQQRSAEPMRLSMDPPMPELQGGQQEQELRQLLNKDKSKRSAVASSVVKQKLAEVILKKQQAALERTVHPSSPSIPYRTLEPLDTEGAARSVLSSFLPPVPSLPTEPPEHFPLRKTVSEPNLKLRYKPKKSLERRKNPLLRKESAPPSLRRRPAETLGDSSPSSSSTPASGCSSPNDSEHGPNPALGSEADGDRRTHSTLGPRGPVLGNPHAPLFLHHGLEPEAGGTLPSRLQPILLLDPSVSHAPLWTVPGLGPLPFHFAQPLLTTERLSGSGLHRPLNRTRSEPLPPSATASPLLAPLQPRQDRLKPHVQLIKPAISPPQRPAKPSEKPRLRQIPSAEDLETDGGGVGPMANDGLEHRESGRGPPEGRGSISLQQHQQVPPWEQQHLAGRLSQGSPGDSVLIPLAQVGHRPLSRTQSSPAAPVSMLSPEPTCQTQVLNSSETPATGLVYDSVMLKHQCSCGDNSKHPEHAGRIQSIWSRLQERGLRSQCECLRGRKASLEELQSVHSERHVLLYGTNPLSRLKLDNGKLTGLLAQRTFVMLPCGGVGVDTDTIWNELHSSNAARWAAGSVTDLAFKVASRELKNGFAVVRPPGHHADHSTAMGFCFFNSVAIACRQLQQHGKASKILIVDWDVHHGNGTQQTFYQDPSVLYISLHRHDDGNFFPGSGAVDEVGTGSGEGFNVNVAWAGGLDPPMGDPEYLAAFRIVVMPIAREFAPDLVLVSAGFDAAEGHPAPLGGYHVSAKCFGYMTQQLMNLAGGAVVLALEGGHDLTAICDASEACVAALLGNKVDPLSEESWKQKPNLSAIRSLEAVVRVHRKYWGCMQRLASCPDSWLPRVPGADAEVEAVTALASLSVGILAEDRPSERLVEEEEPMNL

SEQ ID NO: 13 - protein, 916 aa, Mus musculus
MDLRVGQRPTVEPPPEPALLTLQHPQRLHRHLFLAGLHQQQRSAEPMRLSMDPPMPELQGGQQEQELRQLLNKDKSKRSAVASSVVKQKLAEVILKKQQAALERTVHPSSPSIPYRTLEPLDTEGAARSVLSSFLPPVPSLPTEPPEHFPLRKTVSEPNLKLRYKPKKSLERRKNPLLRKESAPPSLRRRPAETLGDSSPSSSSTPASGCSSPNDSEHGPNPALGSEADGDRRTHSTLGPRGPVLGNPHAPLFLHHGLEPEAGGTLPSRLQPILLLDPSVSHAPLWTVPGLGPLPFHFAQPLLTTERLSGSGLHRPLNRTRSEPLPPSATASPLLAPLQPRQDRLKPHVQLIKPAISPPQRPAKPSEKPRLRQIPSAEDLETDGGGVGPMANDGLEHRESGRGPPEGRGSISLQQHQQVPPWEQQHLAGRLSQGSPGDSVLIPLAQVGHRPLSRTQSSPAAPVSMLSPEPTCQTQVLNSSETPATGLVYDSVMLKHQCSCGDNSKHPEHAGRIQSIWSRLQERGLRSQCECLRGRKASLEELQSVHSERHVLLYGTNPLSRLKLDNGKLTGLLAQRTFVMLPCGGVGVDTDTIWNELHSSNAARWAAGSVTDLAFKVASRELKNGFAVVRPPGHHADHSTAMGFCFFNSVAIACRQLQQHGKASKILIVDWDVHHGNGTQQTFYQDPSVLYISLHRHDDGNFFPGSGAVDEVGTGSGEGFNVNVAWAGGLDPPMGDPEYLAAFRIVVMPIAREFAPDLVLVSAGFDAAEGHPAPLGGYHVSAKCFGYMTQQLMNLAGGAVVLALEGGHDLTAICDASEACVAALLGNKVDPLSEESWKQKPNLSAIRSLEAVVRVHRKYWGCMQRLASCPDSWLPRVPGADAEVEAVTALASLSVGILAEDRPSERLVEEEEPMNL

SEQ ID NO: 14 - protein, 916 aa, synthetic construct
MDLRVGQRPTVEPPPEPALLTLQHPQRLHRHLFLAGLHQQQRSAEPMRLSMDPPMPELQGGQQEQELRQLLNKDKSKRSAVASSVVKQKLAEVILKKQQAALERTVHPSSPSIPYRTLEPLDTEGAARSVLSSFLPPVPSLPTEPPEHFPLRKTVSEPNLKLRYKPKKSLERRKNPLLRKESAPPSLRRRPAETLGDSSPSSSSTPASGCSSPNDSEHGPNPALGSEADGDRRTHSTLGPRGPVLGNPHAPLFLHHGLEPEAGGTLPSRLQPILLLDPSVSHAPLWTVPGLGPLPFHFAQPLLTTERLSGSGLHRPLNRTRSEPLPPSATASPLLAPLQPRQDRLKPHVQLIKPAISPPQRPAKPSEKPRLRQIPSAEDLETDGGGVGPMANDGLEHRESGRGPPEGRGSISLQQHQQVPPWEQQHLAGRLSQGSPGDSVLIPLAQVGHRPLSRTQSSPAAPVSMLSPEPTCQTQVLNSSETPATGLVYDSVMLKHQCSCGDNSKHPEHAGRIQSIWSRLQERGLRSQCECLRGRKASLEELQSVHSERHVLLYGTNPLSRLKLDNGKLTGLLAQRTFVMLPCGGVGVDTDTIWNELHSSNAARWAAGSVTDLAFKVASRELKNGFAVVRPPGHAADHSTAMGFCFFNSVAIACRQLQQHGKASKILIVDWDVHHGNGTQQTFYQDPSVLYISLHRHDDGNFFPGSGAVDEVGTGSGEGFNVNVAWAGGLDPPMGDPEYLAAFRIVVMPIAREFAPDLVLVSAGFDAAEGHPAPLGGYHVSAKCFGYMTQQLMNLAGGAVVLALEGGHDLTAICDASEACVAALLGNKVDPLSEESWKQKPNLSAIRSLEAVVRVHRKYWGCMQRLASCPDSWLPRVPGADAEVEAVTALASLSVGILAEDRPSERLVEEEEPMNL

SEQ ID NO: 15 - protein, 482 aa, Mus musculus
MDLRVGQRPTVEPPPEPALLTLQHPQRLHRHLFLAGLHQQQRSAEPMRLSMDPPMPELQGGQQEQELRQLLNKDKSKRSAVASSVVKQKLAEVILKKQQAALERTVHPSSPSIPYRTLEPLDTEGAARSVLSSFLPPVPSLPTEPPEHFPLRKTVSEPNLKLRYKPKKSLERRKNPLLRKESAPPSLRRRPAETLGDSSPSSSSTPASGCSSPNDSEHGPNPALGSEADGDRRTHSTLGPRGPVLGNPHAPLFLHHGLEPEAGGTLPSRLQPILLLDPSVSHAPLWTVPGLGPLPFHFAQPLLTTERLSGSGLHRPLNRTRSEPLPPSATASPLLAPLQPRQDRLKPHVQLIKPAISPPQRPAKPSEKPRLRQIPSAEDLETDGGGVGPMANDGLEHRESGRGPPEGRGSISLQQHQQVPPWEQQHLAGRLSQGSPGDSVLIPLAQVGHRPLSRTQSSPAAPVSMLSPEPTCQTQVLNSSET

SEQ ID NO: 16 - DNA, 2973 bp, Homo sapiens
  source 1..2973: mol_type = other DNA
atgcacagccccggcgctgatgggacccaggtgagcccgggtgcccactactgcagccccactggcgcaggctgccccaggccctgtgcagacacaccaggccctcagccgcagcccatggacctgcgggtgggccagcggcccccagtggagcccccaccagagcccacattgctggccctgcagcgtccccagcgcctgcaccaccacctcttcctagcaggcctgcagcagcagcgctcggtggagcccatgaggctctccatggacacgccgatgcccgagttgcaggtgggaccccaggaacaagagctgcggcagcttctccacaaggacaagagcaagcgaagtgctgtagccagcagcgtggtcaagcagaagctagcggaggtgattctgaaaaaacagcaggcggccctagaaagaacagtccatcccaacagccccggcattccctacagaaccctggagcccctggagacggaaggagccacccgctccatgctcagcagctttttgcctcctgttcccagcctgcccagtgaccccccagagcacttccctctgcgcaagacagtctctgagcccaacctgaagctgcgctataagcccaagaagtccctggagcggaggaagaatccactgctccgaaaggagagtgcgccccccagcctccggcggcggcccgcagagaccctcggagactcctccccaagtagtagcagcacgcccgcatcagggtgcagctcccccaatgacagcgagcacggccccaatcccatcctgggctcggaggcgctcttgggccagcggctgcggctgcaggagacttctgtggccccgttcgccttgccgacagtgtccttgctgcccgcaatcactctggggctgcccgcccctgccagggctgacagtgaccgcaggacccatccgactctgggccctcgggggccaatcctggggagcccccacactcccctcttcctgccccatggcttggagcccgaggctgggggcaccttgccctctcgcctgcagcccattctcctcctggacccctcaggctctcatgccccgctgctgactgtgcccgggcttgggcccttgcccttccactttgcccagtccttaatgaccaccgagcggctctctgggtcaggcctccactggccactgagccggactcgctcagagcccctgccccccagtgccaccgctcccccaccgccgggccccatgcagccccgcctggagcagctcaaaactcacgtccaggtgatcaagaggtcagccaagccgagtgagaagccccggctgcggcagataccctcggctgaagacctggagacagatggcgggggaccgggccaggtggtggacgatggcctggagcacagggagctgggccatgggcagcctgaggccagaggccccgctcctctccagcagcaccctcaggtgttgctctgggaacagcagcgactggctgggcggctcccccggggcagcaccggggacactgtgctgcttcctctggcccagggtgggcaccggcctctgtcccgggctcagtcttccccagccgcacctgcctcactgtcagccccagagcctgccagccaggcccgagtcctctccagctcagagacccctgccaggaccctgcccttcaccacagggctgatctatgactcggtcatgctgaagcaccagtgctcctgcggtgacaacagcaggcacccggagcacgccggccgcatccagagcatctggtcccggctgcaggagcgggggctccggagccagtgtgagtgtctccgaggccggaaggcctccctggaagagctgcagtcggtccactctgagcggcacgtgctcctctacggcaccaacccgctcagccgcctcaaactggacaacgggaagctggcagggctcctggcacagcggatgtttgtgatgctgccctgtggtggggttggggtggacactgacaccatctggaatgagcttcattcctccaatgcagcccgctgggccgctggcagtgtcactgacctcgccttcaaagtggcttctcgtgagctaaagaatggtttcgctgtggtgcggcccccaggacaccatgcagatcattcaacagccatgggcttctgcttcttcaactcagtggccatcgcctgccggcagctgcaacagcagagcaaggccagcaagatcctcattgtagactgggacgtgcaccatggcaacggcacccagcaaaccttctaccaagaccccagtgtgctctacatctccctgcatcgccatgacgacggcaacttcttcccggggagtggggctgtggatgaggtaggggctggcagcggtgagggcttcaatgtcaatgtggcctgggctggaggtctggacccccccatgggggatcctgagtacctggctgctttcaggatagtcgtgatgcccatcgcccgagagttctctccagacctagtcctggtgtctgctggatttgatgctgctgagggtcacccggccccactgggtggctaccatgtttctgccaaatgttttggatacatgacgcagcaactgatgaacctggcaggaggcgcagtggtgctggccttggagggtggccatgacctcacagccatctgtgacgcctctgaggcctgtgtggctgctcttctgggtaacagggtggatcccctttcagaagaaggctggaaacagaaacccaacctcaatgccatccgctctctggaggccgtgatccgggtgcacagtaaatactggggctgcatgcagcgcctggcctcctgtccagactcctgggtgcctagagtgccaggggctgacaaagaagaagtggaggcagtgaccgcactggcgtccctctctgtgggcatcctggctgaagataggccctcggagcagctggtggaggaggaagaacctatgaatctc

SEQ ID NO: 17 - DNA, 2856 bp, Homo sapiens
  source 1..2856: mol_type = other DNA
atggacctgcgggtgggccagcggcccccagtggagcccccaccagagcccacattgctggccctgcagcgtccccagcgcctgcaccaccacctcttcctagcaggcctgcagcagcagcgctcggtggagcccatgaggctctccatggacacgccgatgcccgagttgcaggtgggaccccaggaacaagagctgcggcagcttctccacaaggacaagagcaagcgaagtgctgtagccagcagcgtggtcaagcagaagctagcggaggtgattctgaaaaaacagcaggcggccctagaaagaacagtccatcccaacagccccggcattccctacagaaccctggagcccctggagacggaaggagccacccgctccatgctcagcagctttttgcctcctgttcccagcctgcccagtgaccccccagagcacttccctctgcgcaagacagtctctgagcccaacctgaagctgcgctataagcccaagaagtccctggagcggaggaagaatccactgctccgaaaggagagtgcgccccccagcctccggcggcggcccgcagagaccctcggagactcctccccaagtagtagcagcacgcccgcatcagggtgcagctcccccaatgacagcgagcacggccccaatcccatcctgggctcggaggcgctcttgggccagcggctgcggctgcaggagacttctgtggccccgttcgccttgccgacagtgtccttgctgcccgcaatcactctggggctgcccgcccctgccagggctgacagtgaccgcaggacccatccgactctgggccctcgggggccaatcctggggagcccccacactcccctcttcctgccccatggcttggagcccgaggctgggggcaccttgccctctcgcctgcagcccattctcctcctggacccctcaggctctcatgccccgctgctgactgtgcccgggcttgggcccttgcccttccactttgcccagtccttaatgaccaccgagcggctctctgggtcaggcctccactggccactgagccggactcgctcagagcccctgccccccagtgccaccgctcccccaccgccgggccccatgcagccccgcctggagcagctcaaaactcacgtccaggtgatcaagaggtcagccaagccgagtgagaagccccggctgcggcagataccctcggctgaagacctggagacagatggcgggggaccgggccaggtggtggacgatggcctggagcacagggagctgggccatgggcagcctgaggccagaggccccgctcctctccagcagcaccctcaggtgttgctctgggaacagcagcgactggctgggcggctcccccggggcagcaccggggacactgtgctgcttcctctggcccagggtgggcaccggcctctgtcccgggctcagtcttccccagccgcacctgcctcactgtcagccccagagcctgccagccaggcccgagtcctctccagctcagagacccctgccaggaccctgcccttcaccacagggctgatctatgactcggtcatgctgaagcaccagtgctcctgcggtgacaacagcaggcacccggagcacgccggccgcatccagagcatctggtcccggctgcaggagcgggggctccggagccagtgtgagtgtctccgaggccggaaggcctccctggaagagctgcagtcggtccactctgagcggcacgtgctcctctacggcaccaacccgctcagccgcctcaaactggacaacgggaagctggcagggctcctggcacagcggatgtttgtgatgctgccctgtggtggggttggggtggacactgacaccatctggaatgagcttcattcctccaatgcagcccgctgggccgctggcagtgtcactgacctcgccttcaaagtggcttctcgtgagctaaagaatggtttcgctgtggtgcggcccccaggacaccatgcagatcattcaacagccatgggcttctgcttcttcaactcagtggccatcgcctgccggcagctgcaacagcagagcaaggccagcaagatcctcattgtagactgggacgtgcaccatggcaacggcacccagcaaaccttctaccaagaccccagtgtgctctacatctccctgcatcgccatgacgacggcaacttcttcccggggagtggggctgtggatgaggtaggggctggcagcggtgagggcttcaatgtcaatgtggcctgggctggaggtctggacccccccatgggggatcctgagtacctggctgctttcaggatagtcgtgatgcccatcgcccgagagttctctccagacctagtcctggtgtctgctggatttgatgctgctgagggtcacccggccccactgggtggctaccatgtttctgccaaatgttttggatacatgacgcagcaactgatgaacctggcaggaggcgcagtggtgctggccttggagggtggccatgacctcacagccatctgtgacgcctctgaggcctgtgtggctgctcttctgggtaacagggtggatcccctttcagaagaaggctggaaacagaaacccaacctcaatgccatccgctctctggaggccgtgatccgggtgcacagtaaatactggggctgcatgcagcgcctggcctcctgtccagactcctgggtgcctagagtgccaggggctgacaaagaagaagtggaggcagtgaccgcactggcgtccctctctgtgggcatcctggctgaagataggccctcggagcagctggtggaggaggaagaacctatgaatctc

SEQ ID NO: 18 - DNA, 1533 bp, Homo sapiens
  source 1..1533: mol_type = other DNA
atggacctgcgggtgggccagcggcccccagtggagcccccaccagagcccacattgctggccctgcagcgtccccagcgcctgcaccaccacctcttcctagcaggcctgcagcagcagcgctcggtggagcccatgaggctctccatggacacgccgatgcccgagttgcaggtgggaccccaggaacaagagctgcggcagcttctccacaaggacaagagcaagcgaagtgctgtagccagcagcgtggtcaagcagaagctagcggaggtgattctgaaaaaacagcaggcggccctagaaagaacagtccatcccaacagccccggcattccctacagaaccctggagcccctggagacggaaggagccacccgctccatgctcagcagctttttgcctcctgttcccagcctgcccagtgaccccccagagcacttccctctgcgcaagacagtctctgagcccaacctgaagctgcgctataagcccaagaagtccctggagcggaggaagaatccactgctccgaaaggagagtgcgccccccagcctccggcggcggcccgcagagaccctcggagactcctccccaagtagtagcagcacgcccgcatcagggtgcagctcccccaatgacagcgagcacggccccaatcccatcctgggctcggaggcgctcttgggccagcggctgcggctgcaggagacttctgtggccccgttcgccttgccgacagtgtccttgctgcccgcaatcactctggggctgcccgcccctgccagggctgacagtgaccgcaggacccatccgactctgggccctcgggggccaatcctggggagcccccacactcccctcttcctgccccatggcttggagcccgaggctgggggcaccttgccctctcgcctgcagcccattctcctcctggacccctcaggctctcatgccccgctgctgactgtgcccgggcttgggcccttgcccttccactttgcccagtccttaatgaccaccgagcggctctctgggtcaggcctccactggccactgagccggactcgctcagagcccctgccccccagtgccaccgctcccccaccgccgggccccatgcagccccgcctggagcagctcaaaactcacgtccaggtgatcaagaggtcagccaagccgagtgagaagccccggctgcggcagataccctcggctgaagacctggagacagatggcgggggaccgggccaggtggtggacgatggcctggagcacagggagctgggccatgggcagcctgaggccagaggccccgctcctctccagcagcaccctcaggtgttgctctgggaacagcagcgactggctgggcggctcccccggggcagcaccggggacactgtgctgcttcctctggcccagggtgggcaccggcctctgtcccgggctcagtcttccccagccgcacctgcctcactgtcagccccagagcctgccagccaggcccgagtcctctccagctcagagacc

SEQ ID NO: 19 - DNA, 2814 bp, Mus musculus
  source 1..2814: mol_type = other DNA
atgcacagccccggcgcgggctgccctgccctccagccagacacaccaggctctcagccccaacccatggacctgcgggtgggccagcggcccacggtggagcccccaccagagcctgcgctgctgaccctgcaacacccccaacgcctgcaccgccatctcttcctggcaggcttacaccagcaacagcgctcagccgagcccatgaggctctccatggacccaccaatgccggagctgcaggggggacagcaggagcaagaacttcggcaacttctcaataaagacaagagcaagcgaagtgccgtagccagcagtgtggtcaagcagaagctggctgaagtgatcctgaagaaacagcaggcagcccttgagagaacagtccatcccagcagccccagtattccctacagaactcttgagcccttggacacagagggtgctgcccgctccgtgcttagcagcttcctgcctcctgttcccagcctgcccactgaacccccggaacactttcccttgcgtaaaacagtgtctgaacccaacctgaagttgcgctacaaacccaagaaatccctggagagacgcaagaatcccctgctcaggaaggagagtgccccgcccagccttcggaggaggcctgccgagacccttggagattcctcccccagtagtagcagcacacccgcgtcagggtgcagctcccctaatgacagcgagcatggccctaaccctgccctaggctcagaggctgatggtgaccgcaggacccattcaactttaggccctcggggtcctgtactggggaacccccatgctcccctcttcctgcaccacggtctggagccagaggctgggggcaccttaccctctcgcctgcaacccattctcctgctggacccctcagtctctcatgccccactgtggactgtgcctggccttgggcccttgcccttccactttgcccagcccttactgaccaccgagcggctctctgggtcaggcctccatcgaccacttaaccggacccgctcagagcccctgccccccagcgccacagcctcccctctgctggcccccctgcagccccgccaggatcggctcaaacctcacgtccagctgatcaagccagccatctcccctccccagaggcctgccaagcccagtgagaagccccgactgcgacagataccctcggctgaggacctagagacagatggtgggggagtgggacctatggcgaatgatggcctggaacatagggagtcaggccgtgggcctcctgagggcagaggctccatttctctgcagcagcaccaacaggtgccaccctgggagcagcagcatctagccgggcggctctctcagggaagccccggggactccgtgctgatacctctggcccaggttggacaccggcccctgtccagaacccagtcttccccagcagcacctgtttccatgttgagcccagagcccacctgtcagacccaagtcctcaacagctcagagacacctgctacagggctggtctatgactcggtgatgctgaaacaccaatgttcctgtggagacaacagcaagcatcccgagcatgcaggccgcatccagagcatctggtcccggctgcaggaacggggtctccgcagccagtgtgagtgtctccgaggccgaaaggcttccctagaggagctgcagtcagtccactctgaacggcacgtgctcctttacggcacgaacccactcagccgcctcaaactggataacgggaagcttacaggactcctggcacagcggacgtttgtgatgctaccctgtggcggggttggggtcgatactgacaccatctggaacgagctgcattcctccaatgcagcccgctgggctgcgggcagtgtcaccgaccttgccttcaaagtagcttcccgagagctgaagaatggctttgctgtggtgcgacccccgggacaccatgcagatcattctacagccatgggcttctgcttcttcaactccgtggccatcgcctgccgacagctacagcaacacggcaaagccagcaagatcctcattgttgactgggatgttcaccatggcaacggcacacagcagactttctaccaggaccccagtgtgctctacatttcccttcatcgccatgacgacggcaacttcttcccaggcagtggggccgtggatgaggtgggaactgccagtggcgagggcttcaatgtcaacgtggcttgggctgggggcttggatccacccatgggggatcctgagtacctggctgctttcaggatagtggtgatgcccattgcccgagagtttgctccagacctggtcctggtgtctgctgggtttgatgctgcggagggtcacccagccccactgggtggctaccatgtttctgccaaatgttttgggtacatgacgcagcagttgatgaacttggcaggaggcgccgtggtgttggccttagagggtggacatgacctcacggccatctgtgatgcctcggaggcctgtgtagctgctcttctgggcaacaaggtggaccccctttcagaagaaagctggaaacagaaacccaacctcagtgccatccgctcgctggaagctgtggtcagggtgcacaggaaatactggggctgcatgcagcgcttggcctcctgtccagactcctggctacccagagtgccgggagctgatgcagaagtggaagccgtgaccgcgctggcatccctttctgtgggcatcctggctgaagacaggccctcggagcggctggtggaagaggaagaacccatgaacctc

SEQ ID NO: 20 - DNA, 2748 bp, Mus musculus
  source 1..2748: mol_type = other DNA
atggacctgcgggtgggccagcggcccacggtggagcccccaccagagcctgcgctgctgaccctgcaacacccccaacgcctgcaccgccatctcttcctggcaggcttacaccagcaacagcgctcagccgagcccatgaggctctccatggacccaccaatgccggagctgcaggggggacagcaggagcaagaacttcggcaacttctcaataaagacaagagcaagcgaagtgccgtagccagcagtgtggtcaagcagaagctggctgaagtgatcctgaagaaacagcaggcagcccttgagagaacagtccatcccagcagccccagtattccctacagaactcttgagcccttggacacagagggtgctgcccgctccgtgcttagcagcttcctgcctcctgttcccagcctgcccactgaacccccggaacactttcccttgcgtaaaacagtgtctgaacccaacctgaagttgcgctacaaacccaagaaatccctggagagacgcaagaatcccctgctcaggaaggagagtgccccgcccagccttcggaggaggcctgccgagacccttggagattcctcccccagtagtagcagcacacccgcgtcagggtgcagctcccctaatgacagcgagcatggccctaaccctgccctaggctcagaggctgatggtgaccgcaggacccattcaactttaggccctcggggtcctgtactggggaacccccatgctcccctcttcctgcaccacggtctggagccagaggctgggggcaccttaccctctcgcctgcaacccattctcctgctggacccctcagtctctcatgccccactgtggactgtgcctggccttgggcccttgcccttccactttgcccagcccttactgaccaccgagcggctctctgggtcaggcctccatcgaccacttaaccggacccgctcagagcccctgccccccagcgccacagcctcccctctgctggcccccctgcagccccgccaggatcggctcaaacctcacgtccagctgatcaagccagccatctcccctccccagaggcctgccaagcccagtgagaagccccgactgcgacagataccctcggctgaggacctagagacagatggtgggggagtgggacctatggcgaatgatggcctggaacatagggagtcaggccgtgggcctcctgagggcagaggctccatttctctgcagcagcaccaacaggtgccaccctgggagcagcagcatctagccgggcggctctctcagggaagccccggggactccgtgctgatacctctggcccaggttggacaccggcccctgtccagaacccagtcttccccagcagcacctgtctccatgctgagcccagagcccacctgtcagacccaagtcctcaacagctcagagacacctgctacagggctggtctatgactcggtgatgctgaaacaccaatgttcctgtggagacaacagcaagcatcccgagcatgcaggccgcatccagagcatctggtcccggctgcaggaacggggtctccgcagccagtgtgagtgtctccgaggccgaaaggcttccctagaggagctgcagtcagtccactctgaacggcacgtgctcctctacggcacgaacccactcagccgcctcaaactggataacgggaagcttacaggactcctggcacagcggacgtttgtgatgctaccctgtggcggggttggggtcgatactgacaccatctggaacgagctgcattcctccaatgcagcccgctgggctgcgggcagcgtcaccgaccttgccttcaaagtagcttcccgagagctgaagaacggctttgctgtggtgcgacccccgggacaccatgcagatcattctacagccatgggcttctgcttcttcaactccgtggccatcgcctgccgacagctacagcaacacggcaaagccagcaagatcctcattgttgactgggatgttcaccatggcaacggcacacagcagactttctaccaggaccccagtgtgctctacatttcccttcatcgtcatgacgacggcaacttcttcccaggcagtggggccgtggatgaggtgggaactggcagtggcgagggcttcaatgtcaacgtggcttgggctgggggcttggatccacccatgggggatcctgagtacctggctgctttcaggatagtggtgatgcccattgcccgagagtttgctccagacctggtcctggtgtctgctgggtttgatgctgcggagggtcacccagccccactgggtggctaccatgtttctgccaaatgttttgggtacatgacgcagcagttgatgaacttggcaggaggcgccgtggtgttggccttagagggtggacatgacctcacggccatctgtgatgcctcggaggcctgtgtagctgctcttctgggcaacaaggtggaccccctttcagaagaaagctggaaacagaaacccaacctcagtgccatccgctcgctggaagctgtggtcagggtgcacaggaaatactggggctgcatgcagcgcttggcctcctgtccagactcctggctacccagagtgccgggagctgatgcagaagtggaagccgtgaccgcgctggcatccctttctgtgggcatcctggctgaagacaggccctcggagcggctggtggaagaggaagaacccatgaacctc

SEQ ID NO: 21 - DNA, 2747 bp, synthetic construct
  source 1..2747: mol_type = other DNA
atggacctgcgggtgggccagcggcccacggtggagcccccaccagagcctgcgctgctgaccctgcaacacccccaacgcctgcaccgccatctcttcctggcaggcttacaccagcaacagcgctcagccgagcccatgaggctctccatggacccaccaatgccggagctgcaggggggacagcaggagcaagaacttcggcaacttctcaataaagacaagagcaagcgaagtgccgtagccagcagtgtggtcaagcagaagctggctgaagtgatcctgaagaaacagcaggcagcccttgagagaacagtccatcccagcagccccagtattccctacagaactcttgagcccttggacacagagggtgctgcccgctccgtgcttagcagcttcctgcctcctgttcccagcctgcccactgaacccccggaacactttcccttgcgtaaaacagtgtctgaacccaacctgaagttgcgctacaaacccaagaaatccctggagagacgcaagaatcccctgctcaggaaggagagtgccccgcccagccttcggaggaggcctgccgagacccttggagattcctcccccagtagtagcagcacacccgcgtcagggtgcagctcccctaatgacagcgagcatggccctaaccctgccctaggctcagaggctgatggtgaccgcaggacccattcaactttaggccctcggggtcctgtactggggaacccccatgctcccctcttcctgcaccacggtctggagccagaggctgggggcaccttaccctctcgcctgcaacccattctcctgctggacccctcagtctctcatgccccactgtggactgtgcctggccttgggcccttgcccttccactttgcccagcccttactgaccaccgagcggctctctgggtcaggcctccatcgaccacttaaccggacccgctcagagcccctgccccccagcgccacagcctcccctctgctggcccccctgcagccccgccaggatcggctcaaacctcacgtccagctgatcaagccagccatctcccctccccagaggcctgccaagcccagtgagaagccccgactgcgacagataccctcggctgaggacctagagacagatggtgggggagtgggacctatggcgaatgatggcctggaacatagggagtcaggccgtgggcctcctgagggcagaggctccatttctctgcagcagcaccaacaggtgccaccctgggagcagcagcatctagccgggcggctctctcagggaagccccggggactccgtgctgatacctctggcccaggttggacaccggcccctgtccagaacccagtcttccccagcagcacctgtctccatgctgagcccagagcccacctgtcagacccaagtcctcaacagctcagagacacctgctacagggctggtctatgactcggtgatgctgaaacaccaatgttcctgtggagacaacagcaagcatcccgagcatgcaggccgcatccagagcatctggtcccggctgcaggaacggggtctccgcagccagtgtgagtgtctccgaggccgaaaggcttccctagaggagctgcagtcagtccactctgaacggcacgtgctcctctacggcacgaacccactcagccgcctcaaactggataacgggaagcttacaggactcctggcacagcggacgtttgtgatgctaccctgtggcggggttggggtcgatactgacaccatctggaacgagctgcattcctccaatgcagcccgctgggctgcgggcagcgtcaccgaccttgccttcaaagtagcttcccgagagctgaagaacggctttgctgtggtgcgacccccgggacacgctgcagatcattctacagccatgggcttctgcttcttcaactccgtggccatcgcctgccgacagctacagcaacacggcaaagccagcaagatcctcattgttgactgggatgttcaccatggcaacggcacacagcagactttctaccaggaccccagtgtgctctacatttcccttcatcgtcatgacgacggcaacttcttcccaggcagtggggccgtggatgaggtgggaactggcagtggcgagggcttcaatgtcaacgtggcttgggctgggggcttggatccacccatgggggatcctgagtacctggctgctttcaggatagtggtgatgcccattgcccgagagtttgctccagacctggtcctggtgtctgctgggtttgatgctgcggagggtcacccagccccactgggtggctaccatgtttctgccaaatgttttgggtacatgacgcagcagttgatgaacttggcaggaggcgccgtggtgttggccttagagggtggacatgacctcacggccatctgtgatgcctcggaggcctgtgtagctgctcttctgggcaacaaggtggaccccctttcagaagaaagctggaaacagaaacccaacctcagtgccatccgctcgctggaagctgtggtcagggtgcacaggaaatactggggctgcatgcagcgcttggcctcctgtccagactcctggctacccagagtgccgggagctgatgcagaagtggaagccgtgaccgcgctggcatccctttctgtgggcatcctggctgaagacaggccctcggagcggctggtggaagaggaagaacccatgaacct

SEQ ID NO: 22 - DNA, 1446 bp, Mus musculus
  source 1..1446: mol_type = other DNA
atggacctgcgggtgggccagcggcccacggtggagcccccaccagagcctgcgctgctgaccctgcaacacccccaacgcctgcaccgccatctcttcctggcaggcttacaccagcaacagcgctcagccgagcccatgaggctctccatggacccaccaatgccggagctgcaggggggacagcaggagcaagaacttcggcaacttctcaataaagacaagagcaagcgaagtgccgtagccagcagtgtggtcaagcagaagctggctgaagtgatcctgaagaaacagcaggcagcccttgagagaacagtccatcccagcagccccagtattccctacagaactcttgagcccttggacacagagggtgctgcccgctccgtgcttagcagcttcctgcctcctgttcccagcctgcccactgaacccccggaacactttcccttgcgtaaaacagtgtctgaacccaacctgaagttgcgctacaaacccaagaaatccctggagagacgcaagaatcccctgctcaggaaggagagtgccccgcccagccttcggaggaggcctgccgagacccttggagattcctcccccagtagtagcagcacacccgcgtcagggtgcagctcccctaatgacagcgagcatggccctaaccctgccctaggctcagaggctgatggtgaccgcaggacccattcaactttaggccctcggggtcctgtactggggaacccccatgctcccctcttcctgcaccacggtctggagccagaggctgggggcaccttaccctctcgcctgcaacccattctcctgctggacccctcagtctctcatgccccactgtggactgtgcctggccttgggcccttgcccttccactttgcccagcccttactgaccaccgagcggctctctgggtcaggcctccatcgaccacttaaccggacccgctcagagcccctgccccccagcgccacagcctcccctctgctggcccccctgcagccccgccaggatcggctcaaacctcacgtccagctgatcaagccagccatctcccctccccagaggcctgccaagcccagtgagaagccccgactgcgacagataccctcggctgaggacctagagacagatggtgggggagtgggacctatggcgaatgatggcctggaacatagggagtcaggccgtgggcctcctgagggcagaggctccatttctctgcagcagcaccaacaggtgccaccctgggagcagcagcatctagccgggcggctctctcagggaagccccggggactccgtgctgatacctctggcccaggttggacaccggcccctgtccagaacccagtcttccccagcagcacctgtctccatgctgagcccagagcccacctgtcagacccaagtcctcaacagctcagagaca

SEQ ID NO: 23 - protein, 442 aa, Mus musculus
MVLNSSETPATGLVYDSVMLKHQCSCGDNSKHPEHAGRIQSIWSRLQERGLRSQCECLRGRKASLEELQSVHSERHVLLYGTNPLSRLKLDNGKLTGLLAQRTFVMLPCGGVGVDTDTIWNELHSSNAARWAAGSVTDLAFKVASRELKNGFAVVRPPGHHADHSTAMGFCFFNSVAIACRQLQQHGKASKILIVDWDVHHGNGTQQTFYQDPSVLYISLHRHDDGNFFPGSGAVDEVGTGSGEGFNVNVAWAGGLDPPMGDPEYLAAFRIVVMPIAREFAPDLVLVSAGFDAAEGHPAPLGGYHVSAKCFGYMTQQLMNLAGGAVVLALEGGHDLTAICDASEACVAALLGNKVDPLSEESWKQKPNLSAIRSLEAVVRVHRKYWGCMQRLASCPDSWLPRVPGADAEVEAVTALASLSVGILAEDRPSERLVEEEEPMNL

SEQ ID NO: 24 - DNA, 1326 bp, Mus musculus
  source 1..1326: mol_type = other DNA
atggtcctcaacagctcagagacacctgctacagggctggtctatgactcggtgatgctgaaacaccaatgttcctgtggagacaacagcaagcatcccgagcatgcaggccgcatccagagcatctggtcccggctgcaggaacggggtctccgcagccagtgtgagtgtctccgaggccgaaaggcttccctagaggagctgcagtcagtccactctgaacggcacgtgctcctctacggcacgaacccactcagccgcctcaaactggataacgggaagcttacagggctcctggcacagcggacgtttgtgatgctaccctgtggcggggttggggtcgatactgacaccatctggaacgagctgcattcctccaatgcagcccgctgggctgcgggcagcgtcaccgaccttgccttcaaagtagcttcccgagagctgaagaacggctttgctgtggtgcgacccccgggacaccatgcagatcattctacagccatgggcttctgcttcttcaactccgtggccatcgcctgccgacagctacagcaacacggcaaagccagcaagatcctcattgttgactgggatgttcaccatggcaacggcacacagcagactttctaccaggaccccagtgtgctctacatttcccttcatcgtcatgacgacggcaacttcttcccaggcagtggggccgtggatgaggtgggaactggcagtggcgagggcttcaatgtcaacgtggcttgggctgggggcttggatccacccatgggggatcctgagtacctggctgctttcaggatagtggtgatgcccattgcccgagagtttgctccagacctggtcctggtgtctgctgggtttgatgctgcggagggtcacccagccccactgggtggctaccatgtttctgccaaatgttttgggtacatgacgcagcagttgatgaacttggcaggaggcgccgtggtgttggccttagagggtggacatgacctcacggccatctgtgatgcctcggaggcctgtgtagctgctcttctgggcaacaaggtggaccccctttcagaagaaagctggaaacagaaacccaacctcagtgccatccgctcgctggaagctgtggtcagggtgcacaggaaatactggggctgcatgcagcgcttggcctcctgtccagactcctggctacccagagtgccgggagctgatgcagaagtggaagccgtgaccgcgctggcatccctttctgtgggcatcctggctgaagacaggccctcggagcggctggtggaagaggaagaacccatgaacctc